(19) United States
(12) Patent Application Publication
(10) Pub. No.: US 2014/0154783 A1
(43) Pub. Date: Jun. 5, 2014

(54) BIOPROCESSING

(71) Applicants: Anthony Rossomando, Cambridge, MA (US); John M. Maraganore, Cambridge, MA (US); Stuart Pollard, Cambridge, MA (US); David Kocisko, Newton, MA (US); Muthiah Manogaran, Cambridge, MA (US); Todd Borland, Cambridge, MA (US); Shannon Hogan, Cambridge, MA (US)

(72) Inventors: Anthony Rossomando, Cambridge, MA (US); John M. Maraganore, Cambridge, MA (US); Stuart Pollard, Cambridge, MA (US); David Kocisko, Newton, MA (US); Muthiah Manogaran, Cambridge, MA (US); Todd Borland, Cambridge, MA (US); Shannon Hogan, Cambridge, MA (US)

(73) Assignee: Alnylam Pharmaceuticals, Inc., Cambridge, MA (US)

(21) Appl. No.: 13/379,797

(22) PCT Filed: Jul. 6, 2010
(86) PCT No.: PCT/US10/41072

§ 371 Date: Mar. 14, 2012

Related U.S. Application Data
(60) Provisional application No. 61/223,370, filed on Jul. 6, 2009.
(60) Provisional application No. 61/244,868, filed on Sep. 22, 2009.
(60) Provisional application No. 61/293,980, filed on Jan. 11, 2010.
(60) Provisional application No. 61/305,284, filed on Feb. 17, 2010.
(60) Provisional application No. 61/319,589, filed on Mar. 31, 2010.

(51) Int. Cl. C12N 7/00

(52) U.S. Cl. CPC C12N 7/00
USPC 435/239

(57) ABSTRACT

The present invention relates to molecular biology, molecular genetics, and bioprocessing. The embodiments provide for compositions and methods for producing a biological product, such as an immunogenic agent, in an embryonated egg by introducing into the egg a RNA effector molecule capable of modulating expression of a target gene, wherein the modulation enhances production of the biological product in the egg. These methods provide for RNAi-based approaches to optimize the production of biologics from embryonated eggs, such as the production of viral vaccines including seasonal and pandemic flu vaccines. The invention also relates to molecules, reagents, cells, and kits useful for carrying out the methods, and biological products produced by the methods.

### RELATED APPLICATIONS

This application claims the benefit of U.S. Provisional Patent Application No. 61/305,284, filed Feb. 17, 2010, entitled BIOPROCESSING, by Pollard et al.; U.S. Provisional Patent Application No. 61/223,370, filed Jul. 6, 2009, entitled COMPOSITIONS AND METHODS FOR ENHANCING PRODUCTION OF A BIOLOGICAL PRODUCT, by Maraganore et al.; U.S. Provisional Patent Application No. 61/244,868, filed Sep. 22, 2009, entitled COMPOSITIONS AND METHODS FOR ENHANCING PRODUCTION OF A BIOLOGICAL PRODUCT, by Maraganore et al.; U.S. Provisional Patent Application No. 61/293,980, filed Jan. 11, 2010, entitled COMPOSITIONS AND METHODS FOR ENHANCING PRODUCTION OF A BIOLOGICAL PRODUCT, by Rossomando et al.; and U.S. Provisional Patent Application No. 61/319,589, filed Mar. 31, 2010, entitled CELL-BASED BIOPROCESSING, by Rossomando et al.; each of which is incorporated fully herein by reference.

### REFERENCE TO SEQUENCES

The specification includes a Sequence Listing as part of the originally filed subject matter. The sequence listing for SEQ ID NOs 1 to 3,290,939 is provided herein in an electronic format on 4 compact discs (CD-R), labeled “CRF,” “COPY 1,” “COPY 2,” and “COPY 3,” as file name “51058072.TXT,” and is incorporated herein by reference in their entirety in to the present specification.

The instant application contains a “lengthy” Sequence Listing which has been submitted via CD-R in lieu of a printed paper copy, and is hereby incorporated by reference in its entirety. Said CD-Rs, recorded on Jul. 1, 2010, are labeled “CRF,” “Copy 1,” “Copy 2” and “Copy 3”, respectively, and each contains only one identical 774,635 KB file (51058072.TXT).

### FIELD OF THE INVENTION

The present invention relates to molecular biology, molecular genetics, and bioprocessing. More specifically, the present invention provides methods for producing a biological product in an embryonated egg by introducing into the egg a RNA effector molecule capable of modulating expression of a target gene, wherein the modulation enhances production of the biological product in the egg. These methods provide for RNAi-based approaches to optimize the production of biologics from fertilized eggs, such as the production of viral vaccines including seasonal and pandemic flu vaccines. The invention also relates to molecules, reagents, cells, and kits useful for carrying out the methods, and biological products produced by the methods.

### BACKGROUND

Fertilized Gallus gallus domesticus (chicken) eggs have been used in vaccine manufacturing for over 50 years. They are the original single-use bioreactors: inexpensive, easily scalable, and relatively environmentally friendly. Human, avian and hybrid human:avian strains used to make vaccine preparations replicate in the fertilized egg and be harvested from the egg, including from the amniotic fluid subsequent to injection into the egg or amniotic space. More human vaccines are manufactured in embryonated eggs than in any other biological substrate, with more than 250 million doses of inactivated seasonal influenza vaccine distributed to 100 countries annually. Vaccines for yellow fever and many veterinary vaccines are also produced routinely in eggs.

Nevertheless, large-scale vaccine production in eggs poses many challenges, hence significant investments have been directed toward developing cell culture-based alternatives. These new platforms, which include recombinant mammalian cell culture, plant-based vaccines, and E. coli and other microbial-based production systems, have made significant progress, but the best of these efforts are still several steps away from meeting the needs of large-scale vaccine production, such as annual influenza prophylaxis.

Given this situation, eggs are likely to remain an important production platform for flu vaccine production for the foreseeable future. Yet, yields of inactivated flu vaccine can range from one to three doses per egg, depending on the strain, and the seasonality of the industry in temperate regions can leave production capacity idle for half the year. These factors indicate the importance of rapid, high yield vaccine bioprocessing within tight margins.

An example of the limitations of current egg-based bioprocessing arose in 2009: The World Health Organization reported that the novel H1N1 virus injected into embryonated eggs to create the pandemic vaccine did not grow well. Compared to seasonal flu viruses, the H1N1 seed strain grew only 25% to 50% percent as fast. It appeared that a crucial surface protein on the H1N1 virus, hemagglutinin, was unstable in eggs. This threw global vaccine researchers back to ‘square one’ as they rushed to isolate new samples of the virus from infected people and hybridize those fresh strains with a flu virus strain that grows well in eggs.

Further, for example, the H5N1 strain of avian influenza virus is lethal in embryonated chicken eggs. Indeed, highly pathogenic avian strains can not be grown in large quantities in chicken eggs because they are lethal to chick embryos.

Hence, although embryonated eggs remain an important approach to bioprocessing, there is a need for techniques to improve product yield, especially for vaccine bioprocessing.

### SUMMARY

The present invention provides for methods for improving production of a biological product, such as an immunogenic agent, in an embryonated egg, comprising introducing into the egg at least one RNA effector molecule, a portion of which is complementary to at least one nucleic acid-based entity (e.g., a target gene); maintaining the egg for a time sufficient to modulate expression of the at least one nucleic acid-based entity, wherein the modulation of expression improves production of a biological product in the egg; and isolating the biological product from the embryonated egg.

More specifically, some embodiments of the present invention relate to initiating RNA interference in an embryonated egg, before, during or after the viral infection or vector inoculation, to inhibit cellular and/or antiviral processes that compromise the yield and quality of the viral/immunogenic product harvest. For example, an embodiment administers a siRNA or shRNA in naked, conjugated or formulated form (e.g., lipid nanoparticle) that targets an embryonic cell's antiviral pathways (e.g., interferon pathway (IFNB), or IFN receptors (e.g., IFNAR 1), and thereby inhibits the cellular antiviral response and enhances viral replication, and therefore yield of biological product (e.g., viral particles and/or immunogenic agents). This can occur by targeting the chorionallantoic membrane via injection into the amniotic space/fluid and/or other tissues producing the desired biological product.

In some embodiments of the invention, with regard to RNAi formulations, simple (naked siRNA in saline or similar solutions or formulations), conjugated (e.g., cholesterol or other targeting ligands) as well as LNP or alternate polymer formulations or delivery vehicles as well as plasmid or viral vectors for shRNA can be used. In addition, the aforesaid formulations can be co-formulated or incorporated into the virion particles or vector themselves to facilitate delivery or stabilize RNAi materials to the relevant embryonated egg tissues where the virus/vector can produce desired product.

In various embodiments, the RNA effector molecule can comprise siRNA, miRNA, dsRNA, saRNA, shRNA, piRNA, tkRNAi, eiRNA, pdRNA, a gapmer, an antagomir, or a ribozyme. In one embodiment the RNA effector molecule is not shRNA. In one embodiment the RNA effector molecule is a dsRNA.

In one embodiment, the RNA effector molecule can activate a target gene.

In another embodiment, the RNA effector can inhibit a target gene.

In some embodiments, the RNA effector molecule comprises a sense strand and an antisense strand of a double-stranded oligonucleotide in which one strand comprises at least 16 contiguous nucleotides (e.g., 17, nucleotides, 18 nucleotides, or 19 nucleotides). In one embodiment, the antisense strand comprises at least 16 contiguous nucleotides. In one embodiment, the antisense strand comprises at least 17 contiguous nucleotides. In one embodiment, the antisense strand comprises at least 18 contiguous nucleotides. In one embodiment, the antisense strand comprises at least 19 contiguous nucleotides. In one embodiment, the antisense strand further comprises at least one deoxyribonucleotide. In one embodiment, the antisense strand further comprises at least two deoxyribonucleotides. In one embodiment, the antisense strand further comprises two deoxythymidine residues.

In some embodiments, the RNA effector molecule comprises an antisense strand of a double-stranded oligonucleotide in which the antisense strand comprises at least 16 contiguous nucleotides (e.g., 17, nucleotides, 18 nucleotides, or 19 nucleotides). In one embodiment, the antisense strand comprises at least 16 contiguous nucleotides. In one embodiment, the antisense strand comprises at least 17 contiguous nucleotides. In one embodiment, the antisense strand comprises at least 18 contiguous nucleotides. In one embodiment, the antisense strand comprises at least 19 contiguous nucleotides. In one embodiment, the antisense strand further comprises at least one deoxyribonucleotide. In one embodiment, the antisense strand further comprises at least two deoxyribonucleotides. In one embodiment, the antisense strand further comprises two deoxythymidine residues.

In particular embodiments, the target gene is associated with viral sensing, such as TLR3, TLR7, TLR21, RIG-1, LPGP2 and other RIG-1-like receptors, TRIM25, or MAVS/VISA/IPS-1/Gardif; an interferon gene such as IFN-α, IFN-β, IFN-γ; an interferon receptor such as IFNAR1 or IFNR2; and gene associated with interferon signaling such as STAT-1, STAT-2, STAT-3, STAT-4, JAK-1, JAK-2, JAK-3, IRF1, IRF2, IRF3, IRF4, IRF5, IRF6 IRF7, IRF8, IRF 9, or IRF10.

In other particular embodiments, the target gene encodes an interferon-induced protein such as 2′,5′ oligoadenylate synthetases (2-5 OAS), RNaseL (ribonuclease L (2′,5′-oligoisoadenylate synthetase-dependent), dsRNA-dependent protein kinase (PKR) (eukaryotic translation initiation factor 2-kinase 2, EIF2AK2), Mx (MX1 myxovirus (influenza virus) resistance 1, interferon-inducible protein p78), IFITM1, IFITM2, IFITM3, Proinflammatory cytokines, MYD88 (myeloid differentiation primary response gene), or TRIF (toll-like receptor adaptor molecule 1).

In yet other particular embodiments, the target gene is a gene associated with cell proliferation, such as protein kinase CK2 β subunit (CSKN2B); a gene associated with apoptosis, such as Bax, Bak ((BCL2-antagonist/killer 1), LDHA (lactate dehydrogenase A), LDHB, BIK, BAD, BIM, HRK, BCLG, HR, NOXA, PUMA, BOK (BCL2-related ovarian killer), BOO, BCLB, CASP2 (apoptosis-related cysteine peptidase (neural precursor cell expressed, developmentally down-regulated 2)), CASP3 (apoptosis-related cysteine peptidase), CASP6, CASP7, CASP8, CASP9, CASP10, BCL2 (B-cell CLL/lymphoma 2), p53, APAF1, HSP70, TRAIL (TRAIL-LIKE TNF-related apoptosis inducing ligand-like), BCL2L1 (BCL2-like 1), BCL2L13 (BCL2-like 13 (apoptosis facilitator)); BCL2L14 (BCL2-like 14 (apoptosis facilitator)), FASLG (Fas ligand (TNF superfamily, member 6)), DPF2 (D4, zinc and double PHD fingers family 2), AIFM2 (apoptosis-inducing factor mitochondrion-associated 2), AIFM3, STK17A (serine/threonine kinase 17a (apoptosis-inducing)), APITD1 (apoptosis-inducing, TAF9-like domain 1), SIVA1 (apoptosis-inducing factor), FAS (TNF receptor superfamily member 6), TGFβ2 (transforming growth factor β 2), TGFBR1 (transforming growth factor, (3 receptor I), LOC378902 (death domain-containing tumor necrosis factor receptor superfamily member 23), or BCL2A1 (BCL2-related protein A1).

In other particular embodiments, the target is a gene identified through screening, such as PUSL1 (pseudouridylate synthase-like 1), TPST1 (tyrosylprotein sulfotransferase 1), WDR33 (WD repeat domain 33), Nod2, MCT4 (solute carrier family 16, member 3 (monocarboxylic acid transporter 4)), ACRC (acidic repeat containing), AMELY, ATCAY (cerebellar, Cayman type (caytaxin)), ANP32B (acidic (leucine-rich) nuclear phosphoprotein 32 family member), DEFA3, DHRS10, DOCK4 (dedicator of cytokinesis 4), FAM106A, FKBP1B (FK506 binding protein 1B), IRF3, KBTBD8 (kelch repeat and BTB (POZ) domain containing 8), KIAA0753 (homolog of KIAA0753 gene), LPGAT1 (lysophosphatidylglycerol acyltransferase 1), MSMB (microseminoprotein β), NFS1 (nitrogen fixation 1 homolog), NPIP, NPM3 (nucleophosmin/nucleoplasmin 3), SCGB2A1, SERPINB7, SLC16A4 (solute carrier family 16, member 4 (monocarboxylic acid transporter 5)), SPTBN4 (spectrin, β, non-erythrocytic 4), or TMEM146; a gene associated with cell cycle/cell proliferation, such as CDKN1B (cyclin-dependent kinase inhibitor 1B, p27, kip1), CDKN2A, or FOXO1.

In some embodiments, the target is PTEN or FN1, or other genes such as miRNA antagonists, host sialidase, NEU2 sialidase 2 (cytosolic sialidase), NEU3 sialidase 3 (membrane sialidase), Dicer (dicer 1, ribonuclease type III), or ISRE (Interferon-stimulated response element).

In other embodiments, the target can be a target inhibiting other host cellular or viral processes that compromise yield and/or quality of product. For example, target genes removing sialic acid from the cell surface to reduce virus binding such as SLC35A1, SLC35A2, GNE, Cmas, B4GalT1, and B4GalT6. Other target genes include the promoter, 3′UTR and/or 5′UTR regions of any of the foregoing.

In other embodiments, the target gene can be an endogenous virus, latent virus, or adventitious virus that can contaminate product, or otherwise compromise yield and/or quality of product. For example, target genes of endogenous retrovirus can be gg01-chr7-7163462, gg01-chrU-52190725, gg01-Chr4-48130894, avian leukosis virus (ALV)pol, ALV p2, ALV p10, ALV env, ALV transmembrane protein, tm, ALV trans-acting factor, gg01-chr1-15168845, gg01-chr4-77338201, gg01-ChrU-163504869, and gg01-chr7-5733782. Target genes of latent DNA viruses can be, for example, genes of an adenovirus-associated virus (AAV). Target genes of adventitious virus can be, for example, genes of ALV.

Additionally, viral progeny can be attenuated by targeting viral proteins associated with virulence (e.g., influenza NP, PA, PB1, PB2, M, and NS genes). The glycosylation pattern of biologic product of interest, such as the hemagglutinin (HA) and neuraminidase (NA) influenza proteins, can be attenuated to improve antigenicity or host adaptation: these target genes share only rare homology between strains, however. In essence, any aspect of the quality and attributes as well efficiency of bioprocessing can be modified by this approach.

Thus, in some embodiments of the present invention, the biological product is a virus, which virus includes naturally occurring virus strains, variants or mutant strains; mutagenized viruses (e.g., generated by exposure to mutagens, repeated passages and/or passage in non-permissive hosts); reassortants (in the case of segmented viral genomes); and/or genetically engineered viruses (e.g., using “reverse genetics” techniques) having the desired phenotype; and other virus-based (viral) products. The viruses of the invention can be attenuated; i.e., they are infectious and can replicate in vivo, but generate low titers resulting in subclinical levels of infection that are non-pathogenic.

Another embodiment further comprises preparing the viral product for use in a vaccine.

In one embodiment, the embryonated egg is administered a plurality of different RNA effector molecules to modulate expression of multiple target genes. The RNA effector molecules can be administered at different times or simultaneously, at the same frequency or different frequencies, at the same concentration or at different concentrations.

In further embodiments, the methods further comprise administering to the embryonated egg with a second agent. The second agent can be an immunosuppressive agent; a growth factor; an apoptosis inhibitor; a kinase inhibitor; a phosphatase inhibitor; a protease inhibitor; an inhibitor of pathogens (e.g., where a virus is the biological product, an agent that inhibits growth and/or propagation of endogenous or contaminating viruses, or fungal or bacterial pathogens); or a histone demethylating agent.

In additional embodiments, the target gene encodes a protein that affects a physiological process of the embryonated egg. In various embodiments, the physiological process is apoptosis, cell cycle progression, carbon metabolism or transport, lactate formation, or RNAi uptake and/or efficacy.

In one embodiment, the invention provides for an embryonated egg containing at least one RNA effector molecule provided herein. The embryonated egg is, for example, an avian egg, a reptilian egg, a fish egg, an insect egg, or an amphibian egg. An avian egg can be a chicken egg, duck egg, turkey egg, goose egg, ostrich egg, or other avian egg.

In another embodiment, the invention provides a composition for enhancing production of a biological product in a cultured, embryonated egg by modulating the expression of a target gene in an egg. The composition typically includes one or more RNA effector molecules described herein and a suitable carrier or delivery vehicle.

In another embodiment, a composition containing two or more RNA effector molecules directed against separate target genes is used to enhance production of a vial product in an embryonated egg by modulating expression of a first target gene and at least a second target gene in the egg, wherein the first and second target gene can be associated with expression of the same protein (e.g., the first target is a coding region and the second target is an UTR).

Still another embodiment of the invention encompasses kits for enhancing production of a biological product in a cultured, embryonated egg. In one aspect, a kit comprises a RNA effector molecule that modulates expression of a target gene encoding a protein that affects production of the biological product. In another embodiment, a kit comprises an embryonated egg that expresses a RNA effector molecule that modulates expression of a protein that affects production of the biological product. Such kits can also comprise instructions for carrying out methods provided herein.

In some embodiments of the present invention, the RNA effector molecule is combined with a sialic acid “decoy” that interacts with influenza virus HA and/or NA residues.

In one embodiment, the sialic acid decoy is introduced to an embryonated egg concurrent with introduction of the RNA effector molecule(s) and infective virus. This avoids multiple exposures of the egg to possible contamination, but provides for a lag in viral infection while the RNA effector(s) contact the cell and modulate cellular activity.

In alternative embodiments, the binding of virus to sialic acid is used as a delivery mechanism to contact RNAi agents (e.g., RNA effector molecules) with the host cell. In this approach, RNA effector molecules are combined with or conjugated to sialic acids or derivatives thereof. In one embodiment, viral inoculum is mixed with RNA-effector-coupled sialic acid derivatives, such that a portion of the hemagglutinin residues on the virus are complexed with sialic acid-siRNA conjugate.

In other embodiments, sialic acids are incorporated into liposomal formulations with the siRNA. In an aspect of this embodiment, the siRNA-sialic acid-liposome formulation is mixed with influenza prior to inoculation of the embryonated egg.

In some embodiments of the present invention, it is advantageous to temporarily inhibit viral replication, for example, until the host cell immune response is at least partially inhibited (e.g., by the RAN effector molecule), such that viral replication ensues after adequate suppression of the cell immune response. In an alternative approach, cells are inoculated with virus, unbound virus is washed from the cells, and these infected cells are then introduced to the embryonated egg concurrent with the RNA effector molecule. In an alternative embodiment, the viral multiplicity of infection (MOI) can be relatively low compared to the RNA effector molecules and the cell density in the embryonated egg, thus allowing greater influence of the RNA effector in the egg cells as the viral titer builds.

Eggs account for approximately 50% of bulk vaccine cost in inactivated influenza vaccine manufacturing. The better yield and quality of RNA interference-based approaches offer a way of lowering vaccine cost by reducing the number of eggs required for a given yield. For example, a 10-fold increase in yield could result in a 40% reduction in vaccine cost. In addition, by producing more vaccine per unit operating time, inactivated influenza vaccine production can be accelerated. Even a 3- to 5-fold reduction in the number of eggs required would dramatically improve manufacturing logistics and have the follow-on effect of enhancing quality control, as well as expand the vaccine supply in epidemic and pandemic outbreaks of diseases such as influenza.

### DETAILED DESCRIPTION

This invention is not limited to the particular methodology, protocols, and reagents, etc., described herein and as such can vary. The terminology used herein is for the purpose of describing particular embodiments only, and is not intended to limit the scope of the present invention, which is defined solely by the claims.

As used herein and in the claims, the singular forms include the plural reference and vice versa unless the context clearly indicates otherwise. Other than in the operating examples, or where otherwise indicated, all numbers expressing quantities of ingredients or reaction conditions used herein should be understood as modified in all instances by the term “about.”

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials can be used in the practice or testing of the invention, the methods, devices, and materials in this regard are described herein.

Although human gene symbols are typically designated by upper-case letters, in the present specification the use of either upper-case or lower-case gene symbols can be used interchangeably and include both human or non-human species. Thus, for example, a reference in the specification to the gene or gene target “lactate dehydrogenase A” as “LDHA” (“Ldha” or “LdhA”), includes human and/or non-human (e.g., avian) genes and gene targets. In other words, the upper-case or lower-case letters in a particular gene symbol do not limit the scope of the gene or gene target to human or non-human species. All gene identification numbers provided herein (GeneID) are those of the domestic chicken, Gallus gallus, from the National Center for Biotechnology Information “Entrez Gene” web site unless identified otherwise.

### I. DEFINITIONS

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used herein, “immunogenic agent” refers to an agent used to stimulate the immune system of a subject, so that one or more functions of the immune system are increased and directed towards the immunogenic agent. An antigen or immunogen is intended to mean a molecule containing one or more epitopes that can stimulate a host immune system to make a secretory, humoral and/or cellular immune response specific to that antigen Immunogenic agents can be used in the production of antibodies, both isolated polyclonal antibodies and monoclonal antibodies, using techniques known in the art. Immunogenic agents include vaccines.

As used herein, “vaccine” refers to an agent used to stimulate the immune system of a subject so that protection is provided against an antigen not recognized as a self-antigen by the subject's immune system. Immunization refers to the process of inducing a high level of antibody and/or cellular immune response in a subject, that is directed against a pathogen or antigen to which the organism has been exposed. Vaccines and immunogenic agents as used herein, refer to a subject's immune system: the anatomical features and mechanisms by which a subject produces antibodies and/or cellular immune responses against an antigenic material that invades the subject's cells or extra-cellular fluids. In the case of antibody production, the antibody so produced can belong to any of the immunological classes, such as immunoglobulins, A, D, E, G, or M. Vaccines that stimulate production of immunoglobulin A (IgA) are of interest, because IgA is the principal immunoglobulinof the secretory system in warm-blooded animals. Vaccines are likely to produce a broad range of other immune responses in addition to IgA formation, for example cellular and humoral immunity Immune responses to antigens are well-studied and reported widely. See, e.g., Elgert, IMMUNOL. (Wiley Liss, Inc., 1996); Stites et al., BASIC & CLIN. IMMUNOL., (7th Ed., Appleton & Lange, 1991). By contrast, the phrase “immune response of the host cell” refers to the responses of unicellular host organisms (i.e., cells within the embryonated egg to the presence of foreign bodies.

In the context of this invention, the term “oligonucleotide” or “nucleic acid molecule” encompasses not only nucleic acid molecules as expressed or found in nature, but also analogs and derivatives of nucleic acids comprising one or more ribo- or deoxyribo-nucleotide/nucleoside analogs or derivatives as described herein or as known in the art. Such modified or substituted oligonucleotides are often used over native forms because of properties such as, for example, enhanced cellular uptake, increased stability in the presence of nucleases, and the like, discussed further herein. A “nucleoside” includes a nucleoside base and a ribose sugar, and a “nucleotide” is a nucleoside with one, two or three phosphate moieties. The terms “nucleoside” and “nucleotide” can be considered to be equivalent as used herein. An oligonucleotide can be modified in the nucleobase structure or in the ribose-phosphate backbone structure, e.g., as described herein, including the modification of a RNA nucleotide into a DNA nucleotide. The molecules comprising nucleoside analogs or derivatives must retain the ability to form a duplex.

As non-limiting examples, an oligonucleotide can also include at least one modified nucleoside including but not limited to a 2′-O-methyl modified nucleoside, a nucleoside comprising a 5′ phosphorothioate group, a terminal nucleoside linked to a cholesterol derivative or dodecanoic acid bisdecylamide group, a locked nucleoside, an abasic nucleoside, a 2′-deoxy-2′-fluoro modified nucleoside, a 2′-amino-modified nucleoside, 2′-alkyl-modified nucleoside, morpholino nucleoside, a phosphoramidate or a non-natural base comprising nucleoside, or any combination thereof. Alternatively, an oligonucleotide can comprise at least two modified nucleosides, at least 3, at least 4, at least 5, at least 6, at least 7, at least 8, at least 9, at least 10, at least 15, at least 20, or more, up to the entire length of the oligonucleotide. The modifications need not be the same for each of such a plurality of modified nucleosides in an oligonucleotide. When RNA effector molecule is double stranded, each strand can be independently modified as to number, type and/or location of the modified nucleosides. In one embodiment, modified oligonucleotides contemplated for use in methods and compositions described herein are peptide nucleic acids (PNAs) that have the ability to form the required duplex structure and that permit or mediate the specific degradation of a target RNA via a RISC pathway.

The terms “ribonucleoside”, “ribonucleotide”, “nucleotide”, or “deoxyribonucleotide” can also refer to a modified nucleotide, as further detailed herein, or a surrogate replacement moiety. A ribonucleotide comprising a thymine base is also referred to as 5-methyl uridine and a deoxyribonucleotide comprising a uracil base is also referred to as deoxy-Uridine in the art. Guanine, cytosine, adenine, thymine and uracil can be replaced by other moieties without substantially altering the base pairing properties of an oligonucleotide comprising a nucleotide bearing such replacement moiety. For example, without limitation, a nucleotide comprising inosine as its base can base pair with nucleotides containing adenine, cytosine, or uracil. Hence, nucleotides containing uracil, guanine, or adenine can be replaced in the nucleotide sequences of dsRNA featured in the invention by a nucleotide containing, for example, inosine. In another example, adenine and cytosine anywhere in the oligonucleotide can be replaced with guanine and uracil, respectively to form G-U Wobble base pairing with the target mRNA. Sequences containing such replacement moieties are suitable for the compositions and methods featured in the invention.

Similarly, the skilled artisan will recognize that the term “RNA molecule” or “ribonucleic acid molecule” encompasses not only RNA molecules as expressed or found in nature, but also analogs and derivatives of RNA comprising one or more ribonucleotide or ribonucleoside analogs or derivatives as described herein or as known in the art. The terms “ribonucleoside” and “ribonucleotide” can be considered to be equivalent as used herein. The RNA can be modified in the nucleobase structure or in the ribose-phosphate backbone structure, e.g., as described herein.

In one aspect, a RNA effector molecule can include a deoxyribonucleoside residue. In such an instance, a RNA effector molecule agent can comprise one or more deoxynucleosides, including, for example, a deoxynucleoside overhang(s), or one or more deoxynucleosides within the double stranded portion of a dsRNA.

In some embodiments, a plurality of RNA effector molecules is used to modulate expression of one or more target genes. A “plurality” refers to at least 2 or more RNA effector molecules e.g., 3, 4, 5, 6, 7, 8, 9, 10, 11, 12, 13, 14, 15, 16, 17, 18, 19, 20, 30, 40, 50, 60, 80, 100 RNA effector molecules or more. “Plurality” can also refer to at least 2 or more target genes, e.g., 2, 3, 4, 5, 6, 7, 8, 9, 10, 11, 12, 13, 14, 15, 16, 17, 18, 19, 20, 30, 40, 50, 60, 70, 80, 90, 100 target genes or more.

As used herein the term “contacting a host cell” refers to the treatment of a host cell within an egg (“egg cell” or “host egg cell”) with an agent such that the agent is introduced into a cell. The host cell is within the embryonated egg, such that using at least one RNA effector molecule (e.g., a siRNA), often prepared in a composition comprising a delivery agent that facilitates RNA effector uptake into the cell e.g., to contact the cell in the by inolculating the composition into the egg. In one embodiment, the host egg cell is contacted with a vector that encodes an RNA effector molecule, e.g., an integrating or non-integrating vector. In one embodiment, the cell is contacted with a vector that encodes a RNA effector molecule prior to infecting the egg for viral production. In one embodiment, contacting a host egg cell does not include contacting a host cell with a vector the encodes a RNA effector molecule prior to infecting the host cell for viral production, i.e. the cell is contacted with an RNA effector molecule only during production, e.g., added to the egg during the process of producing a biological product. In one embodiment contacting a host egg cell does not include contacting the host cell with a vector that encodes an RNA effector molecule.

The step of contacting a host egg cell with a RNA effector molecule(s) can be repeated more than once (e.g., twice, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100× or more).

In one embodiment, the cell is contacted such that the target gene is modulated only transiently, e.g., by addition of a RNA effector molecule composition to the egg used for the production of a biological product where the presence of the RNA effector molecules dissipates over time, i.e., the RNA effector molecule is not constitutively expressed in the cell.

“Introducing into a cell”, when referring to a RNA effector molecule, means facilitating or effecting uptake or absorption into the egg host cell, as is understood by those skilled in the art. Absorption or uptake of a RNA effector molecule can occur through unaided diffusive or active cellular processes, or by auxiliary agents or devices. For example, introducing into a cell means contacting a egg cell with at least one RNA effector molecule, or means the treatment of a cell with at least one RNA effector molecule and an agent that facilitates or effects uptake or absorption into the cell, often prepared in a composition comprising the RNA effector molecule and delivery agent that facilitates RNA effector molecule uptake (e.g., a transfection reagent, an emulsion, a cationic lipid, a non-cationic lipid, a charged lipid, a liposome, an anionic lipid, a penetration enhancer, or a modification to the RNA effector molecule to attach, e.g., a ligand, a targeting moiety, a peptide, a lipophillic group etc.). In vitro introduction into a cell includes methods known in the art such as electroporation and lipofection. Further approaches are described herein below or known in the art.

In some embodiments, the RNA effector molecule is a siRNA or shRNA effector molecule introduced into an egg cell by introducing into the egg an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of Listeria, Shigella, Salmonella, E. coli, or Bifidobacteriae, or a non-invasive bacterium that has been genetically modified to increase its invasive properties, e.g., by introducing one or more genes that enable invasive bacteria to access the cytoplasm of egg cells. Examples of such cytoplasm-targeting genes include listeriolysin O of Listeria and the invasin protein of Yersinia pseudotuberculosis. Methods for delivering RNA effector molecules to animal cells to induce transkingdom RNAi (tkRNAi) are known in the art. See, e.g., U.S. Patent Pub. No. 2008/0311081 and No. 2009/0123426. In one embodiment, the RNA effector molecule is a siRNA molecule. In one embodiment, the RNA effector molecule is not a shRNA molecule.

As used herein, a “RNA effector composition” includes an effective amount of a RNA effector molecule and an acceptable carrier. As used herein, “effective amount” refers to that amount of a RNA effector molecule effective to produce an effect (e.g., modulatory effect) on a bioprocess for the production of a biological product. In one embodiment, the RNA effector composition comprises a reagent that facilitates RNA effector molecule uptake (e.g., a transfection reagent, an emulsion, a cationic lipid, a non-cationic lipid, a charged lipid, a liposome, an anionic lipid, a penetration enhancer, or a modification to the RNA effector molecule to attach (e.g., a ligand, a targeting moiety, a peptide, a lipophillic group, etc.).

The term “acceptable carrier” refers to a carrier for administration of a RNA effector molecule to host egg cells. Such carriers include, but are not limited to, saline, buffered saline, dextrose, water, glycerol, ethanol, and combinations thereof. In one embodiment the term “acceptable carrier” specifically excludes cell culture medium.

The term “expression” as used herein is intended to mean the transcription to a RNA and/or translation to one or more polypeptides from a target gene coding for the sequence of the RNA and/or the polypeptide.

As used herein, “target gene” refers to a gene that encodes a protein that affects one or more aspects of the production of a biological product by a host cell, such that modulating expression of the gene enhances production of the biological product. Target genes can be derived from the host cell, endogenous to the host cell (present in the host cell genome), transgenes (gene constructs inserted at ectopic sites in the host cell genome), or derived from a pathogen (e.g., a virus, fungus or bacterium) that is capable of infecting the host cell or the subject who will use the biological product or derivatives thereof (e.g., humans).

In some embodiments, the target gene is an endogenous gene of the egg cell. For example, in particular embodiments the target gene can encode a polypeptide or protein. The target gene can also encode a host cell protein that directly or indirectly affects one or more aspects of the production of the viral product. Examples of target genes that affect the production of viral polypeptides include genes encoding proteins involved in the secretion, folding or post-translational modification of polypeptides (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, susceptibility to viral infection or RNAi uptake, activity or efficacy); and genes encoding proteins that impair the production of a biological product by the host cell (e.g., a protein that binds or co-purifies with the biological product).

Additionally, in some embodiments, a “target gene” refers to a gene that regulates expression of a nucleic acid (i.e., non-encoding genes) that affects one or more aspects of the production of a biological product by a cell, such that modulating expression of the gene enhances production of the biological product.

By “target gene RNA” or “target RNA” is meant RNA transcribed from the target gene. Hence, a target gene can be a coding region, a promoter region, a 3′ untranslated region (3′-UTR), and/or a 5′-UTR of the target gene.

A target gene RNA that encodes a polypeptide is more commonly known as messenger RNA (mRNA). Target genes can be derived from the host cell, latent in the host cell, endogenous to the host cell (present in the host cell genome), transgenes (gene constructs inserted at ectopic sites in the host cell genome), or derived from a pathogen (e.g., a virus, fungus or bacterium) which is capable of infecting either the host cell or the subject who will use the a biological product or derivatives or products thereof. In some embodiments, the target gene encodes a protein that affects one or more aspects of post-translational modification, e.g., peptide glycosylation, by a host cell. For example, modulating expression of a gene encoding a protein involved in post-translational processing enhances production of a polypeptide comprising at least one terminal mannose.

In some embodiments, the target gene encodes a non-coding RNA (ncRNA), such as an untranslated region. As used herein, a ncRNA refers to a target gene RNA that is not translated into a protein. The ncRNA can also be referred to as non-protein-coding RNA (npcRNA), non-messenger RNA (nmRNA), small non-messenger RNA (snmRNA), and functional RNA (fRNA) in the art. The target gene from which a ncRNA is transcribed as the end product is also referred to as a RNA gene or ncRNA gene. ncRNA genes include highly abundant and functionally important RNAs such as transfer RNA (tRNA) and ribosomal RNA (rRNA), as well as RNAs such as snoRNAs, microRNAs, siRNAs, and piRNAs. As used herein, a RNA effector molecule is said to target within a particular site of a RNA transcript if the RNA effector molecule promotes cleavage of the transcript anywhere within that particular site.

In some embodiments, the target gene is an endogenous gene of the host cell. For example, the target gene can encode the immunogenic agent or a portion thereof when the immunogenic agent is a polypeptide. The target gene can also encode a host cell protein that directly or indirectly affects one or more aspects of the production of the immunogenic agent. Examples of target genes that affect the production of polypeptides include genes encoding proteins involved in the secretion, folding or post-translational modification of polypeptides (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, cytoskeletal structure (e.g., actin dynamics), susceptibility to viral infection or RNAi uptake, activity or efficacy); and genes encoding proteins that impair the production of an immunogenic agent by the host cell (e.g., a protein that binds or co-purifies with the immunogenic agent).

In some embodiments, the target gene encodes a host cell protein that indirectly affects the production of the immunogenic agent such that inhibiting expression of the target gene enhances production of the immunogenic agent. For example, the target gene can encode an abundantly expressed host cell protein that does not directly influence production of the immunogenic agent, but indirectly decreases its production, for example by utilizing cellular resources that could otherwise enhance production of the immunogenic agent. Target genes are discussed in more detail herein.

The term “modulates expression of” and the like, in so far as it refers to a target gene, herein refers to the modulation of expression of a target gene, as manifested by a change (e.g., an increase or a decrease) in the amount of target gene mRNA that can be isolated from or detected in a first cell or group of cells in which a target gene is transcribed and that has or have been treated such that the expression of a target gene is modulated, as compared to a second cell or group of cells substantially identical to the first cell or group of cells but that has or have not been so treated (control cells). The degree of modulation can be expressed in terms of:

$$\frac{( mRNA in control cells ) - ( mRNA in treated cells )}{( mRNA in control cells )} \cdot 100 \%$$

Alternatively, the degree of modulation can be given in terms of a parameter that is functionally linked to target gene expression, e.g., the amount of protein encoded by a target gene, or the number of cells displaying a certain phenotype, e.g., stabilization of microtubules. In principle, target gene modulation can be determined in any host cell expressing the target gene, either constitutively or by genomic engineering, and by any appropriate assay known in the art.

For example, in certain instances, expression of a target gene is inhibited. For example, expression of a target gene is inhibited by at least about 10%, 15%, 20%, 25%, 30%, 35%, 40%, 45%, or 50% by administration of an RNA effector molecule provided herein. In some embodiments, a target gene is inhibited by at least about 60%, 70%, or 80% by administration of a RNA effector molecule. In some embodiments, a target gene is inhibited by at least about 85%, 90%, or 95% or more by administration of a RNA effector molecule as described herein. In other instances, expression of a target gene is activated by at least about 10%, 20%, 25%, 50%, 100%, 200%, 400% or more by administration of a RNA effector molecule provided herein.

As used herein, the term “RNA effector molecule” refers to an oligonucleotide agent capable of modulating the expression of a target gene, as defined herein, within a host cell, or a oligonucleotide agent capable of forming such an oligonucleotide, optionally, within a host cell (i.e., upon being introduced into a host cell). A portion of a RNA effector molecule is substantially complementary to at least a portion of the target gene, such as the coding region, the promoter region, the 3′ untranslated region (3′-UTR), and/or the 5′-UTR of the target gene.

The RNA effector molecules described herein generally have a first strand and a second strand, one of which is substantially complementary to at least a portion of the target gene and modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and pre-translational mechanisms.

RNA effector molecules can comprise a single strand or more than one strand, and can include, e.g., double stranded RNA (dsRNA), microRNA (miRNA), antisense RNA, promoter-directed RNA (pdRNA), Piwi-interacting RNA (piRNA), expressed interfering RNA (eiRNA), short hairpin RNA (shRNA), antagomirs, decoy RNA, DNA, plasmids, and aptamers. The RNA effector molecule can be single-stranded or double-stranded. A single-stranded RNA effector molecule can have double-stranded regions and a double-stranded RNA effector can have single-stranded regions.

The term “portion”, when used in reference to an oligonucleotide (e.g., a RNA effector molecule), refers to a portion of a RNA effector molecule having a desired length to effect complementary binding to a region of a target gene, or a desired length of a duplex region. For example, a “portion” or “region” refers to a nucleic acid sequence of at least 3, at least 4, at least 5, at least 6, at least 7, at least 8, at least 9, at least 10 or more nucleotides up to one nucleotide shorter than the entire RNA effector molecule. In some embodiments, the “region” or “portion” when used in reference to a RNA effector molecule includes nucleic acid sequence one nucleotide shorter than the entire nucleic acid sequence of a strand of an RNA effector molecule. One of skill in the art can vary the length of the “portion” that is complementary to the target gene or arranged in a duplex, such that a RNA effector molecule having desired characteristics (e.g., inhibition of a target gene or stability) is produced. Although not bound by theory, RNA effector molecules provided herein can modulate expression of target genes by one or more of a variety of mechanisms, including but not limited to, Argonaute-mediated post-transcriptional cleavage of target gene mRNA transcripts (sometimes referred to in the art as RNAi) and/or other pre-transcriptional and/or pre-translational mechanisms.

RNA effector molecules disclosed herein include a RNA strand (the antisense strand) having a region which is 30 nucleotides or less in length, e.g., 10 to 30 nucleotides in length, or 19 to 24 nucleotides in length, which region is substantially complementary to at least a portion of a target gene that affects one or more aspects of the production of a biological product, such as the yield, purity, homogeneity, biological activity, or stability of the biological product. A RNA effector molecule can comprise a sense strand and an antisense strand, wherein one strand comprises at least 16 contiguous nucleotides (e.g., at least 17, at least 18, at least 19 nucleotides) of the nucleotides of an siRNA sequence provided for herein. The RNA effector molecules interact with RNA transcripts of target genes and mediate their selective degradation or otherwise prevent their translation.

The term “antisense strand” refers to the strand of a RNA effector molecule, e.g., a dsRNA, which includes a region that is substantially complementary to a target sequence. The term “region of complementarity” refers to the region on the antisense strand that is substantially complementary to a sequence, for example a target sequence, as defined herein. Where the region of complementarity is not fully complementary to the target sequence, the mismatches can be in the internal or terminal regions of the molecule. Generally, the most tolerated mismatches are in the terminal regions, e.g., within 5, 4, 3, or 2 nucleotides of the 5′ and/or 3′ terminus.

The term “sense strand” refers to the strand of an RNA effector molecule that includes a region that is substantially complementary to a region of the antisense strand as that term is defined herein.

As used herein, and unless otherwise indicated, the term “complementary”, when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as understood by the skilled artisan. “Complementary” sequences can also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but are not limited to, G:U Wobble or Hoogstein base pairing. Hybridization conditions can, for example, be stringent conditions, where stringent conditions can include 400 mM NaCl, 40 mM PIPES pH 6.4, 1 mM EDTA, 50° C. or 70° C., for 12 to 16 hours followed by washing. Other conditions, such as physiologically relevant conditions as can be encountered inside an organism, can apply. The skilled artisan will be able to determine the set of conditions most appropriate for a test of complementarity of two sequences in accordance with the ultimate application of the hybridized nucleotides.

The terms “complementary,” “fully complementary” and “substantially complementary” herein can be used with respect to the base matching between the sense strand and the antisense strand of a dsRNA, or between the antisense strand of an RNA effector molecule agent and a target sequence, as will be understood from the context of use. As used herein, an oligonucleotide that is “substantially complementary to at least part of” a target gene refers to an oligonucleotide that is substantially complementary to a contiguous portion of a target gene of interest (e.g., a mRNA encoded by a target gene, the target gene's promoter region or 3′ UTR, or ERV LTR). For example, an oligonucleotide is complementary to at least a part of a target mRNA if the sequence is substantially complementary to a non-interrupted portion of an mRNA encoded by a target gene.

Complementary sequences within a RNA effector molecule, e.g., within a dsRNA (a double-stranded ribonucleic acid) as described herein, include base-pairing of the oligonucleotide or polynucleotide comprising a first nucleotide sequence to an oligonucleotide or polynucleotide comprising a second nucleotide sequence over the entire length of one or both nucleotide sequences. Such sequences can be referred to as “fully complementary” with respect to each other herein. Where a first sequence is referred to as “substantially complementary” with respect to a second sequence herein, the two sequences can be fully complementary, or they can form one or more, but generally not more than 5, 4, 3 or 2 mismatched base pairs upon hybridization for a duplex up to 30 base pairs, while retaining the ability to hybridize under the conditions most relevant to their ultimate application, e.g., inhibition of gene expression via a RISC pathway. Where two oligonucleotides are designed to form, upon hybridization, one or more single-stranded overhangs, such overhangs shall not be regarded as mismatches with regard to the determination of complementarity. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, can yet be referred to as “fully complementary” for the purposes described herein.

In some embodiments, the RNA effector molecule comprises a single-stranded oligonucleotide that interacts with and directs the cleavage of RNA transcripts of a target gene. For example, single stranded RNA effector molecules comprise a 5′ modification including one or more phosphate groups or analogs thereof to protect the effector molecule from nuclease degradation. The RNA effector molecule can be a single-stranded antisense nucleic acid having a nucleotide sequence that is complementary to at least a portion of a “sense” nucleic acid of a target gene, e.g., the coding strand of a double-stranded cDNA molecule or an RNA sequence, e.g., a pre-mRNA, mRNA, miRNA, or pre-miRNA. Accordingly, an antisense nucleic acid can form hydrogen bonds with a sense nucleic acid target. In an alternative embodiment, the RNA effector molecule comprises a duplex region of at least nine nucleotides in length.

Given a coding strand sequence (e.g., the sequence of a sense strand of a cDNA molecule), antisense nucleic acids can be designed according to the rules of Watson-Crick base pairing. The antisense nucleic acid can be complementary to a portion of the coding or noncoding region of a RNA, e.g., the region surrounding the translation start site of a pre-mRNA or mRNA, e.g., the 5′ UTR. An antisense oligonucleotide can be, for example, about 10 to 25 nucleotides in length (e.g., 10, 11, 12, 13, 14, 15, 16, 18, 19, 20, 21, 22, 23, or 24 nucleotides in length). In some embodiments, the antisense oligonucleotide comprises one or more modified nucleotides, e.g., phosphorothioate derivatives and/or acridine substituted nucleotides, designed to increase its biological stability of the molecule and/or the physical stability of the duplexes formed between the antisense and target nucleic acids. Antisense oligonucleotides can comprise ribonucleotides only, deoxyribonucleotides only (e.g., oligodeoxynucleotides), or both deoxyribonucleotides and ribonucleotides. For example, an antisense agent consisting only of ribonucleotides can hybridize to a complementary RNA and prevent access of the translation machinery to the target RNA transcript, thereby preventing protein synthesis. An antisense molecule including only deoxyribonucleotides, or deoxyribonucleotides and ribonucleotides, can hybridize to a complementary RNA and the RNA target can be subsequently cleaved by an enzyme, e.g., RNAse H, to prevent translation. The flanking RNA sequences can include 2′-O-methylated nucleotides, and phosphorothioate linkages, and the internal DNA sequence can include phosphorothioate internucleotide linkages. The internal DNA sequence is preferably at least five nucleotides in length when targeting by RNAseH activity is desired.

In some embodiments, RNA effector molecule is a double-stranded oligonucleotide. The term “double-stranded RNA” or “dsRNA”, as used herein, refers to an oligonulceotide molecule or complex of molecules having a hybridized duplex region that comprises two anti-parallel and substantially complementary nucleic acid strands, which will be referred to as having “sense” and “antisense” orientations with respect to a target RNA. Typically, region of complementarity is 30 nucleotides or less in length, generally, for example, 10 to 26 nucleotides in length, 18 to 25 nucleotides in length, or 19 to 24 nucleotides in length, inclusive. Upon contact with a cell expressing the target gene, the RNA effector molecule inhibits the expression of the target gene by at least 10% as assayed by, for example, a PCR or branched DNA (bDNA)-based method, or by a protein-based method, such as by protein immunoblot. Expression of a target gene in the egg cells can be assayed by measuring target gene mRNA levels, e.g., by bDNA or TAQMAN® assay, or by measuring protein levels, e.g., by immunofluorescence analysis or quantitative immunoblot.

The duplex region can be of any length that permits specific degradation of a desired target RNA through a RISC pathway, but will typically range from 9 to 36 base pairs in length, e.g., 15 to 30 base pairs in length. More specifically, the duplex region can be of any length that permits specific degradation of a desired target RNA through a RISC pathway, but will typically range from 9 to 36 base pairs in length, e.g., 15 to 30 base pairs in length. Considering a duplex between 9 and 36 base pairs, the duplex can be any length in this range, for example, 9, 10, 11, 12, 13, 14, 15, 16, 17, 18, 19, 20, 21, 22, 23, 24, 25, 26, 27, 28, 29, 30, 31, 32, 33, 34, 35, or 36 and any sub-range there between, including, but not limited to 15 to 30 base pairs, 15 to 26 base pairs, 15 to 23 base pairs, 15 to 22 base pairs, 15 to 21 base pairs, 15 to 20 base pairs, 15 to 19 base pairs, 15 to 18 base pairs, 15 to 17 base pairs, 18 to 30 base pairs, 18 to 26 base pairs, 18 to 23 base pairs, 18 to 22 base pairs, 18 to 21 base pairs, 18 to 20 base pairs, 19 to 30 base pairs, 19 to 26 base pairs, 19 to 23 base pairs, 19 to 22 base pairs, 19 to 21 base pairs, 19 to 20 base pairs, 20 to 30 base pairs, 20 to 26 base pairs, 20 to 25 base pairs, 20 to 24 base pairs, 20 to 23 base pairs, 20 to 22 base pairs, 20 to 21 base pairs, 21 to 30 base pairs, 21 to 26 base pairs, 21 to 25 base pairs, 21 to 24 base pairs, 21 to 23 base pairs, or 21 to 22 base pairs, inclusive.

dsRNAs generated in the cell by processing with Dicer and similar enzymes are generally in the range of 19 to 22 base pairs in length. One strand of the duplex region of a dsDNA comprises a sequence that is substantially complementary to a region of a target RNA. The two strands forming the duplex structure can be from a single RNA molecule having at least one self-complementary region, or can be formed from two or more separate RNA molecules. Where the duplex region is formed from two strands of a single molecule, the molecule can have a duplex region separated by a single stranded chain of nucleotides (a “hairpin loop”) between the 3′-end of one strand and the 5′-end of the respective other strand forming the duplex structure. The hairpin loop can comprise at least one unpaired nucleotide; in some embodiments the hairpin loop can comprise at least 3, at least 4, at least 5, at least 6, at least 7, at least 8, at least 9, at least 10, at least 20, at least 23 or more unpaired nucleotides. Where the two substantially complementary strands of a dsRNA are comprised by separate RNA molecules, those molecules need not, but can be covalently connected. Where the two strands are connected covalently by means other than a hairpin loop, the connecting structure is referred to as a “linker.” The term “sRNA effector molecule” is also used herein to refer to a dsRNA.

Described herein are RNA effector molecules that modulate expression of a target gene. In one embodiment, the RNA effector molecule agent includes double-stranded ribonucleic acid (dsRNA) molecules for inhibiting the expression of a target gene in a cell, where the dsRNA includes an antisense strand having a region of complementarity which is complementary to at least a part of a target gene formed in the expression of a target gene, and where the region of complementarity is 30 nucleotides or less in length, generally 10 to 24 nucleotides in length, and where the dsRNA, upon contact with an cell expressing the target gene, inhibits the expression of the target gene by at least 10% as assayed by, for example, a PCR, PERT, or bDNA-based method, or by a protein-based method, such as a protein immunoblot (e.g., a western blot). Expression of a target gene in an cell can be assayed by measuring target gene mRNA levels, e.g., by PERT, bDNA or TAQMAN® gene expression assay, or by measuring protein levels, e.g., by immunofluorescence analysis or quantitative protein immunoblot.

A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence, derived, for example, from the sequence of an mRNA formed during the expression of a target gene. The other strand (the sense strand) includes a region that is complementary to the antisense strand, such that the two strands hybridize and form a duplex structure when combined under suitable conditions. Generally, the duplex structure is, for example between 9 and 36, between 10 to 30 base pairs, between 18 and 25, between 19 and 24, or between 19 and 21 base pairs in length, inclusive. Similarly, the region of complementarity to the target sequence is, for example, between 10 and 30, between 18 and 25, between 19 and 24, or between 19 and 21 nucleotides in length, inclusive. In some embodiments, the dsRNA is between 10 and 20 nucleotides in length, inclusive, and in other embodiments, the dsRNA is between 25 and 30 nucleotides in length, inclusive. Thus, in one embodiment, to the extent that it becomes processed to a functional duplex of e.g., 15 to 30 base pairs that targets a desired RNA for cleavage, an RNA molecule or complex of RNA molecules having a duplex region greater than 30 base pairs is a dsRNA. As the ordinarily skilled person will recognize, the targeted region of an RNA targeted for cleavage will most often be part of a larger RNA molecule, often a mRNA molecule.

Where relevant, a “part” of a mRNA target is a contiguous sequence of a mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 10 nucleotides in length, such as from 15 to 30 nucleotides in length, inclusive.

The skilled person is well aware that dsRNAs having a duplex structure of between 20 and 23, but specifically 21, base pairs have been hailed as particularly effective in inducing RNA interference. Elbashir et al., 20 EMBO 6877-88 (2001). In the embodiments described above, by virtue of the nature of the oligonucleotide sequences, dsRNAs described herein can include at least one strand of a length of 21 nucloetides. It can be reasonably expected that shorter duplexes having one of the sequences minus only a few nucleotides on one or both ends can be similarly effective as compared to the dsRNAs described in detail. Hence, dsRNAs having a partial sequence of at least 10, 11, 12, 13, 14, 15, 16, 17, 18, 19, 20, or more contiguous nucleotides from a given sequence, inclusive, and differing in their ability to inhibit the expression of a target gene by 5%, 10%, 15%, 20%, 25%, or 30% inhibition, inclusive, from a dsRNA comprising the full sequence, are contemplated according to the invention.

The dsRNA can be synthesized by standard methods known in the art as further discussed below, e.g., by use of an automated DNA synthesizer, such as are commercially available from, for example, Biosearch Technologies (Novato, Calif.). In one embodiment, a target gene is a human target gene. In specific embodiments, the first sequence is a sense strand of a dsRNA that includes a sense sequence and the second sequence is a strand of a ds RNA that includes an antisense sequence. Alternative dsRNA agents that target elsewhere in the target sequence can readily be determined using the target sequence and the flanking target sequence. In this aspect, one of the two sequences is complementary to the other of the two sequences, with one of the sequences being substantially complementary to a sequence of an mRNA generated in the expression of a target gene. As such, in this aspect, a dsRNA will include two oligonucleotides, where one oligonucleotide is described as the sense strand and the second oligonucleotide is described as the antisense strand. As described elsewhere herein and as known in the art, the complementary sequences of a dsRNA can also be contained as self-complementary regions of a single nucleic acid molecule, as opposed to being on separate oligonucleotides.

A double-stranded oligonucleotide can include one or more single-stranded nucleotide overhangs. As used herein, the term “nucleotide overhang” refers to at least one unpaired nucleotide that protrudes from the terminus of a duplex structure of a double-stranded oligonucleotide, e.g., a dsRNA. For example, when a 3′-end of one strand of double-stranded oligonucleotide extends beyond the 5′-end of the other strand, or vice versa, there is a nucleotide overhang. A double-stranded oligonucleotide can comprise an overhang of at least one nucleotide; alternatively the overhang can comprise at least two nucleotides, at least three nucleotides, at least four nucleotides, at least five nucleotides or more. A nucleotide overhang can comprise or consist of a nucleotide/nucleoside analog. The overhang(s) can be on the sense strand, the antisense strand or any combination thereof. Furthermore, the nucleotide(s) of an overhang can be present on the 5′ end, 3′ end, or both ends of either an antisense or sense strand of a dsRNA.

In one embodiment, at least one end of a dsRNA has a single-stranded nucleotide overhang of 1 to 4, generally 1 or 2 nucleotides. For example, a siRNA can two deoxythymidines (or “dTdT”) at each 3′ end. In other words, each strand of a dsRNA can have a two nucleotide overhang at the 3′ end, each comprising a DNA dinucleotide. dsRNAs having at least one nucleotide overhang have unexpectedly superior inhibitory properties relative to their blunt-ended counterparts. Moreover, the presence of a nucleotide overhang on only one strand, at one end of a dsRNA, strengthens the interference activity of the dsRNA, without affecting its overall stability. Such an overhang need not be a single nucleotide overhang; a dinucleotide overhang can also be present.

The antisense strand of a double-stranded oligonucleotide has a 1 to 10 nucleotide overhang at the 3′ end and/or the 5′ end, such as a double-stranded oligonucleotide having a 1 to 10 nucleotide overhang at the 3′ end and/or the 5′ end. One or more of the internucloside linkages in the overhang can be replaced with a phosphorothioate. In some embodiments, the overhang comprises one or more deoxyribonucleoside or the overhang comprises one or more dT, e.g., the sequence 5′-dTdT-3′ or 5′-dTdTdT-3′. In some embodiments, overhang comprises the sequence 5′-dT*dT-3, wherein * is a phosphorothioate internucleoside linkage.

Without being bound theory, double-stranded oligonucleotides having at least one nucleotide overhang have unexpectedly superior inhibitory properties relative to their blunt-ended counterparts. Moreover, the presence of a nucleotide overhang on only one strand, at one end of a dsRNA, strengthens the interference activity of the double-stranded oligonucleotide, without affecting its overall stability.

dsRNA having only one overhang has proven particularly stable and effective in vivo, as well as in a variety of cells, cell culture media, blood, serum, and embryonated eggs. Generally, the single-stranded overhang is located at the 3′-terminal end of an antisense strand or, alternatively, at the 3′-terminal end of a sense strand. The dsRNA having an overhang on only one end will also have one blunt end, generally located at the 5′-end of the antisense strand. Such dsRNAs have superior stability and inhibitory activity, thus allowing administration at low dosages, i.e., less than 5 mg/kg body weight of the recipient per day. In one embodiment, the antisense strand of a dsRNA has a 1 to 10 nucleotide overhang at the 3′ end and/or the 5′ end. In one embodiment, the sense strand of a dsRNA has a 1 to 10 nucleotide overhang at the 3′ end and/or the 5′ end. In another embodiment, one or more of the nucleotides in the overhang is replaced with a nucleoside thiophosphate.

The terms “blunt” or “blunt ended” as used herein in reference to double-stranded oligonucleotide mean that there are no unpaired nucleotides or nucleotide analogs at a given terminal end of a double-stranded oligonucleotide, i.e., no nucleotide overhang. One or both ends of a double-stranded oligonucleotide can be blunt. Where both ends are blunt, the oligonucleotide is said to be double-blunt ended. To be clear, a “double-blunt ended” oligonucleotide is a double-stranded oligonucleotide that is blunt at both ends, i.e., no nucleotide overhang at either end of the molecule. Most often such a molecule will be double-stranded over its entire length. When only one end of is blunt, the oligonucleotide is said to be single-blunt ended. To be clear, a “single-blunt ended” oligonucleotide is a double-stranded oligonucleotide that is blunt at only one end, i.e., no nucleotide overhang at one end of the molecule. Generally, a single-blunt ended oligonucleotide is blunt ended at the 5′-end of sense stand.

A RNA effector molecule as described herein can contain one or more mismatches to the target sequence. For example, a RNA effector molecule as described herein contains no more than three mismatches. If the antisense strand of the RNA effector molecule contains mismatches to a target sequence, it is preferable that the area of mismatch not be located in the center of the region of complementarity. If the antisense strand of the RNA effector molecule contains mismatches to the target sequence, it is preferable that the mismatch be restricted to be within the last 5 nucleotides from either the 5′ or 3′ end of the region of complementarity. For example, for a 23-nucleotide RNA effector molecule agent RNA strand which is complementary to a region of a target gene, the RNA strand generally does not contain any mismatch within the central 13 nucleotides. The methods described herein, or methods known in the art, can be used to determine whether a RNA effector molecule containing a mismatch to a target sequence is effective in inhibiting the expression of a target gene. Consideration of the efficacy of RNA effector molecules with mismatches in inhibiting expression of a target gene is important, especially if the particular region of complementarity in a target gene is known to have polymorphic sequence variation within the population.

In some embodiments, the RNA effector molecule is a promoter-directed RNA (pdRNA) which is substantially complementary to at least a portion of a noncoding region of an mRNA transcript of a target gene. In one embodiment, the pdRNA is substantially complementary to at least a portion of the promoter region of a target gene mRNA at a site located upstream from the transcription start site, e.g., more than 100, more than 200, or more than 1,000 bases upstream from the transcription start site. In another embodiment, the pdRNA is substantially complementary to at least a portion of the 3′-UTR of a target gene mRNA transcript. In one embodiment, the pdRNA comprises dsRNA of 18-28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′ MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, it is believed that pdRNAs modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known, see, e.g., WO 2009/046397.

In some embodiments, the RNA effector molecule comprises an aptamer which binds to a non-nucleic acid ligand, such as a small organic molecule or protein, e.g., a transcription or translation factor, and subsequently modifies (e.g., inhibits) activity. An aptamer can fold into a specific structure that directs the recognition of a targeted binding site on the non-nucleic acid ligand. Aptamers can contain any of the modifications described herein.

In some embodiments, the RNA effector molecule comprises an antagomir. Antagomirs are single stranded, double stranded, partially double stranded or hairpin structures that target a microRNA. An antagomir consists essentially of or comprises at least 10 or more contiguous nucleotides substantially complementary to an endogenous miRNA and more particularly a target sequence of an miRNA or pre-miRNA nucleotide sequence. Antagomirs preferably have a nucleotide sequence sufficiently complementary to a miRNA target sequence of about 12 to 25 nucleotides, such as about 15 to 23 nucleotides, to allow the antagomir to hybridize to the target sequence. More preferably, the target sequence differs by no more than 1, 2, or 3 nucleotides from the sequence of the antagomir. In some embodiments, the antagomir includes a non-nucleotide moiety, e.g., a cholesterol moiety, which can be attached, e.g., to the 3′ or 5′ end of the oligonucleotide agent.

In some embodiments, antagomirs are stabilized against nucleolytic degradation by the incorporation of a modification, e.g., a nucleotide modification. For example, in some embodiments, antagomirs contain a phosphorothioate comprising at least the first, second, and/or third internucleotide linkages at the 5′ or 3′ end of the nucleotide sequence. In further embodiments, antagomirs include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). In some embodiments, antagomirs include at least one 2′-O-methyl-modified nucleotide.

In some embodiments, the RNA effector molecule is a promoter-directed RNA (pdRNA) which is substantially complementary to at least a portion of a noncoding region of an mRNA transcript of a target gene. The pdRNA can be substantially complementary to at least a portion of the promoter region of a target gene mRNA at a site located upstream from the transcription start site, e.g., more than 100, more than 200, or more than 1,000 bases upstream from the transcription start site. Also, the pdRNA can substantially complementary to at least a portion of the 3′-UTR of a target gene mRNA transcript. For example, the pdRNA comprises dsRNA of 18 to 28 bases optionally having 3′ di- or tri-nucleotide overhangs on each strand. The dsRNA is substantially complementary to at least a portion of the promoter region or the 3′-UTR region of a target gene mRNA transcript. In another embodiment, the pdRNA comprises a gapmer consisting of a single stranded polynucleotide comprising a DNA sequence which is substantially complementary to at least a portion of the promoter or the 3′-UTR of a target gene mRNA transcript, and flanking the polynucleotide sequences (e.g., comprising the 5 terminal bases at each of the 5′ and 3′ ends of the gapmer) comprising one or more modified nucleotides, such as 2′MOE, 2′OMe, or Locked Nucleic Acid bases (LNA), which protect the gapmer from cellular nucleases.

pdRNA can be used to selectively increase, decrease, or otherwise modulate expression of a target gene. Without being limited to theory, pdRNAs can modulate expression of target genes by binding to endogenous antisense RNA transcripts which overlap with noncoding regions of a target gene mRNA transcript, and recruiting Argonaute proteins (in the case of dsRNA) or host cell nucleases (e.g., RNase H) (in the case of gapmers) to selectively degrade the endogenous antisense RNAs. In some embodiments, the endogenous antisense RNA negatively regulates expression of the target gene and the pdRNA effector molecule activates expression of the target gene. Thus, in some embodiments, pdRNAs can be used to selectively activate the expression of a target gene by inhibiting the negative regulation of target gene expression by endogenous antisense RNA. Methods for identifying antisense transcripts encoded by promoter sequences of target genes and for making and using promoter-directed RNAs are known. See, e.g., WO 2009/046397.

Expressed interfering RNA (eiRNA) can be used to selectively increase, decrease, or otherwise modulate expression of a target gene. Typically, eiRNA, the dsRNA is expressed in the first transfected cell from an expression vector. In such a vector, the sense strand and the antisense strand of the dsRNA can be transcribed from the same nucleic acid sequence using e.g., two convergent promoters at either end of the nucleic acid sequence or separate promoters transcribing either a sense or antisense sequence. Alternatively, two plasmids can be cotransfected, with one of the plasmids designed to transcribe one strand of the dsRNA while the other is designed to transcribe the other strand. Methods for making and using eiRNA effector molecules are known in the art. See, e.g., WO 2006/033756; U.S. Patent Pubs. No. 2005/0239728 and No. 2006/0035344.

In some embodiments, the RNA effector molecule comprises a small single-stranded Piwi-interacting RNA (piRNA effector molecule) which is substantially complementary to at least a portion of a target gene, as defined herein, and which selectively binds to proteins of the Piwi or Aubergine subclasses of Argonaute proteins. Without being limited to a particular theory, it is believed that piRNA effector molecules interact with RNA transcripts of target genes and recruit Piwi and/or Aubergine proteins to form a ribonucleoprotein (RNP) complex that induces transcriptional and/or post-transcriptional gene silencing of target genes. A piRNA effector molecule can be about 10 to 50 nucleotides in length, about 25 to 39 nucleotides in length, or about 26 to 31 nucleotides in length. See, e.g., U.S. Patent Pub. No. 2009/0062228.

MicroRNAs are a highly conserved class of small RNA molecules that are transcribed from DNA in the genomes of plants and animals, but are not translated into protein. Pre-microRNAs are processed into miRNAs. Processed microRNAs are single stranded ˜17 to 25 nucleotide RNA molecules that become incorporated into the RNA-induced silencing complex (RISC) and have been identified as key regulators of development, cell proliferation, apoptosis and differentiation. They are believed to play a role in regulation of gene expression by binding to the 3′-untranslated region of specific mRNAs. MicroRNAs cause post-transcriptional silencing of specific target genes, e.g., by inhibiting translation or initiating degradation of the targeted mRNA. In some embodiments, the miRNA is completely complementary with the target nucleic acid. In other embodiments, the miRNA has a region of noncomplementarity with the target nucleic acid, resulting in a “bulge” at the region of non-complementarity. In some embodiments, the region of noncomplementarity (the bulge) is flanked by regions of sufficient complementarity, e.g., complete complementarity, to allow duplex formation. For example, the regions of complementarity are at least 8 to 10 nucleotides long (e.g., 8, 9, or 10 nucleotides long).

miRNA can inhibit gene expression by, e.g., repressing translation, such as when the miRNA is not completely complementary to the target nucleic acid, or by causing target RNA degradation, when the miRNA binds its target with perfect or a high degree of complementarity. In further embodiments, the RNA effector molecule can include an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene. The oligonucleotide agent can include naturally occurring nucleobases, sugars, and covalent internucleotide (backbone) linkages and/or oligonucleotides having one or more non-naturally-occurring features that confer desirable properties, such as enhanced cellular uptake, enhanced affinity for the endogenous miRNA target, and/or increased stability in the presence of nucleases. In some embodiments, an oligonucleotide agent designed to bind to a specific endogenous miRNA has substantial complementarity, e.g., at least 70%, 80%, 90%, or 100% complementary, with at least 10, 20, or 25 or more bases of the target miRNA. Exemplary oligonucleiotde agents that target miRNAs and pre-miRNAs are described, for example, in U.S. Patent Pubs. No. 2009/0317907, No. 2009/0298174, No. 2009/0291907, No. 2009/0291906, No. 2009/0286969, No. 2009/0236225, No. 2009/0221685, No. 2009/0203893, No. 2007/0049547, No. 2005/0261218, No. 2009/0275729, No. 2009/0043082, No. 2007/0287179, No. 2006/0212950, No. 2006/0166910, No. 2005/0227934, No. 2005/0222067, No. 2005/0221490, No. 2005/0221293, No. 2005/0182005, and No. 2005/0059005.

A miRNA or pre-miRNA can be 10 to 200 nucleotides in length, for example from 16 to 80 nucleotides in length. Mature miRNAs can have a length of 16 to 30 nucleotides, such as 21 to 25 nucleotides, particularly 21, 22, 23, 24, or 25 nucleotides in length. miRNA precursors can have a length of 70 to 100 nucleotides and can have a hairpin conformation. In some embodiments, miRNAs are generated in vivo from pre-miRNAs by the enzymes cDicer and Drosha. miRNAs or pre-miRNAs can be synthesized in vivo by a cell-based system or can be chemically synthesized. miRNAs can comprise modifications which impart one or more desired properties, such as superior stability, hybridization thermodynamics with a target nucleic acid, targeting to a particular tissue or cell-type, and/or cell permeability, e.g., by an endocytosis-dependent or -independent mechanism. Modifications can also increase sequence specificity, and consequently decrease off-site targeting.

In further embodiments, the RNA effector molecule can comprise an oligonucleotide agent which targets an endogenous miRNA or pre-miRNA. For example, the RNA effector can target an endogenous miRNA which negatively regulates expression of a target gene, such that the RNA effector alleviates miRNA-based inhibition of the target gene.

As used herein, the phrase “in the presence of at least one RNA effector molecule” encompasses exposure of the cell to a RNA effector molecule expressed within the cell, e.g., shRNA, or exposure by exogenous addition of the RNA effector molecule to the cell, e.g., delivery of the RNA effector molecule to the cell, optionally using an agent that facilitates uptake into the cell. A portion of a RNA effector molecule is substantially complementary to at least a portion of the target gene RNA, such as the coding region, the promoter region, the 3′ untranslated region (3′-UTR), or a long terminal repeat (LTR) of the target gene RNA. RNA effector molecules disclosed herein include a RNA strand (the antisense strand) having a region which is 30 nucleotides or less in length, e.g., 10 to 200 nucleotides in length, or 19 to 24 nucleotides in length, which region is substantially complementary to at least a portion of a target gene which encodes a protein that affects one or more aspects of the production of a biological product, such as the yield, purity, homogeneity, biological activity, or stability of the biological product. A RNA effector molecule interacts with RNA transcripts of a target gene and mediates its selective degradation or otherwise prevents its translation. In various embodiments of the present invention, the RNA effector molecule is at least one gapmer, or siRNA, miRNA, dsRNA, saRNA, shRNA, piRNA, tkRNAi, eiRNA, pdRNA, antagomir, or ribozyme.

Double-stranded and single-stranded oligonucleotides that are effective in inducing RNA interference are also referred to as siRNA, RNAi agent, or iRNA agent, herein. These RNA interference inducing oligonucleotides associate with a cytoplasmic multi-protein complex known as RNAi-induced silencing complex (RISC). Without being bound by theory, RNA interference leads to Argonaute-mediated post-transcriptional cleavage of target gene mRNA transcripts. In many embodiments, single-stranded and double-stranded RNAi agents are sufficiently long that they can be cleaved by an endogenous molecule, e.g., by Dicer, to produce smaller oligonucleotides that can enter the RISC machinery and participate in RISC mediated cleavage of a target sequence, e.g., a target mRNA.

In some embodiments, the RNAs provided herein identify a site in a target transcript that is susceptible to RISC-mediated cleavage. As such, the present invention further features RNA effector molecules that target within one of such sequences. Such an RNA effector molecule will generally include at least 10 contiguous nucleotides from one of the sequences provided coupled to additional nucleotide sequences taken from the region contiguous to the selected sequence in a target gene.

The phrase “genome information” as used herein and throughout the claims and specification is meant to refer to sequence information from partial or entire genome of an organism, including protein coding and non-coding regions. These sequences are present every cell originating from the same organisms. As opposed to the transcriptome sequence information, genome information comprises not only coding regions, but also, for example, intronic sequences, promoter sequences, silencer sequences and enhancer sequences. Thus, the “genome information” can refer to, for example a human genome, a mouse genome, a rat genome. One can use complete genome information or partial genome information to add an additional dimension to the database sequences to increase the potential targets to modify with an RNA effector molecule.

The phrase “play a role” refers to any activity of a transcript or a protein in a molecular pathway known to a skilled artisan or identified elsewhere in this specification. Such pathways an cellular activities include, but are not limited to apoptosis, cell division, glycosylation, growth rate, a cellular productivity, a peak cell density, a sustained cell viability, a rate of ammonia production or consumption, or a rate of lactate production.

A “host cell”, as used herein, is any cell, cell culture, cellular biomass or tissue capable of being grown and maintained in an embryonated egg under conditions allowing for production and recovery of useful quantities of a biological product, e.g., an immunogenic agent. A host cell can be cultured in the egg of an insect, amphibian, fish, reptile, or bird. Host cells can be unmodified or genetically modified (e.g., from a transgenic animal) to facilitate production of a biological product. For example, transgenic chicken eggs can have one or more genes essential for the IFN pathway, e.g., interferon receptor, STAT1, etc., disrupted, i.e., a trangenic “knockout.” See, e.g., Sang, 12 Trends Biotech. 415 (1994); Perry et al., 2 Transgenic Res. 125 (1993); Stern, 212 Curr Top Micro. Immunol. 195-206 (1996); Shuman, 47 Experientia 897 (1991). Also, the host cell can be modified to allow for growth under desired conditions, e.g., incubation at 30° C.

“Isolating biological product from the host cell” means at least one step in separating the biological product away from host cellular material, e.g., the host cell, the egg extracellular milieu, the embryonic biomass, or egg. Thus, isolating biologics that are ultimately harvested from the egg are encompassed in the phrase “isolated from the host cell.” A useful quantity includes an amount, including an aliquot or sample, used to screen for or monitor production, including monitoring modulation of target gene expression.

The present invention provides for the production of piological products including “immunogenic agents”, which includes an antigen, antigenic polypeptide, a metabolite, an intermediate, a viral antigen, bacterial antigen, fungal antigen, parasite antigen, virus particle, defective virus, live attenuated virus, killed virus, or vaccine. Immunogenic agents can include any immunogenic substance capable of being produced by a host cell and recovered in useful quantities, including polypeptides, glycoproteins and “biologics” such as a a vaccine that is synthesized from living organisms or their products, and used as a preventive, or therapeutic agent. Thus, immunogenic agents can be used for a wide range of applications, including as biotherapeutic agents, vaccines, research or diagnostic reagents, and the like.

In some embodiments, the biological product is a polypeptide. The polypeptide can be a recombinant polypeptide or a polypeptide endogenous to the embryonated egg. In some embodiments, the polypeptide is a glycoprotein. Non-limiting examples of polypeptides that can be produced according to methods provided herein include receptors, membrane proteins, cytokines, chemokines, hormones, enzymes, growth factors, growth factor receptors, antibodies, antibody derivatives and other immune effectors, interleukins, interferons, erythropoietin, integrins, soluble major histocompatibility complex antigens, binding proteins, transcription factors, translation factors, oncoproteins or proto-oncoproteins, muscle proteins, myeloproteins, neuroactive proteins, tumor growth suppressors, structural proteins, and blood proteins (e.g., thrombin, serum albumin, Factor VII, Factor VIII, Factor IX, Factor X, Protein C, von Willebrand factor, etc.).

As used herein, a polypeptide encompasses glycoproteins or other polypeptides which have undergone post-translational modification, such as deamidation, glycosylation, and the like. In some embodiments, the immunogenic agent is an aberrantly glycosylated protein.

In some embodiments, the biologic is an immunogenic agent, e.g., an immunogenic viral, bacterial, protozoan, or recombinant protein derived from an expression vector.

Another approach for producing viral-based vaccines involves the use of attenuated live virus vaccines, which are capable of replication but are not pathogenic, and, therefore, provide lasting immunity and afford greater protection against disease. The conventional methods for producing attenuated viruses involve the chance isolation of host range mutants, many of which are temperature sensitive, e.g., the virus is passaged through unnatural hosts, and progeny viruses which are immunogenic, yet not pathogenic, are selected. Efficient vaccine production requires the growth of large quantities of virus produced in high yields from a host system. Different types of virus require different growth conditions in order to obtain acceptable yields. The host in which the virus is grown is therefore of great significance. As a function of the virus type, an attenuated live virus can be grown in embryonated eggs.

Thus, in some embodiments of the present invention, the immunogenic agent is a viral product, for example, naturally occurring viral strains, variants or mutants; mutagenized viruses (e.g., generated by exposure to mutagens, repeated passages and/or passage in non-permissive hosts), reassortants (in the case of segmented viral genomes), and/or genetically engineered viruses (e.g., using the “reverse genetics” techniques) having the desired phenotype. The viruses of these embodiments can be attenuated; i.e., they are infectious and can replicate in vivo, but generate low titers resulting in subclinical levels of infection that are generally non-pathogenic.

In some embodiments, the enhancement of production of an immunogenic agent is achieved by improving viability of the cells in the egg. As used herein, the term “improving cell viability” refers to an increase in embryonated egg cell density (e.g., as assessed by a Trypan Blue exclusion assay) or a decrease in apoptosis (e.g., as assessed using a TUNEL assay) of at least 10% in the presence of a RNA effector molecule(s) compared with the cell density or apoptosis levels in the egg without such a treatment. In some embodiments, the increase in cell density or decrease in apoptosis in response to treatment with a RNA effector molecule(s) is at least 20%, at least 30%, at least 40%, at least 50%, at least 60%, at least 70%, at least 80%, at least 90%, at least 95%, or even 100% compared to untreated cells. In some embodiments, the increase in cell density in response to treatment with a RNA effector molecule(s) is at least 2-fold, at least 5-fold, at least 10-fold, at least 20-fold, at least 50-fold, at least 100-fold, at least 1000-fold or higher than the cell density in the absence of the RNA effector molecule(s).

“Bioprocessing” as used herein is an exemplary process for the industrial-scale production of biological product (e.g., an immunogenic agent) in and embryonated egg. Several human, mammalian and avian viruses can infect cells of the chorioallantoic membrane of eggs (a monolayer of cells surrounding the fluid-filled allantoic cavity in the egg). Infection results in the accumulation of product, such as live virus particles, in the allantoic fluid which can be collected after a suitable incubation period. The standard method of egg-based vaccine production consists of pre-incubation of the embryonated eggs, inoculation with a live virus (e.g., influenza, yellow fever), incubation, harvesting of allantoic fluids, downstream processing, and filling and finishing. For example, influenza viruses are typically grown during 2 to 4 days at 37° C. in 10 to 11 day-old eggs. For the classic inactivated influenza vaccine, purification, inactivation, and stabilization of this harvested material yields vaccine product, which techniques are well-known in the art.

Although most of the human primary isolates of influenza A and B viruses grow better in the amniotic sac of the embryos, after two to three passages the viruses become adapted to grow in the cells of the allantoic cavity, which is accessible from the outside of the egg. Murphy & Webster, Orthomyxoviruses 1397-1445, in FIELDS VIROLOGY (Lippincott-Raven pub., PA, 1996). Recombinant DNA technology and genetic engineering techniques, in theory, can afford a superior approach to producing an attenuated virus because specific mutations are deliberately engineered into the viral genome. The genetic alterations required for attenuation of viruses are not always predictable, however. In general, the attempts to use recombinant DNA technology to engineer viral vaccines have been directed to the production of subunit vaccines which contain only the protein subunits of the pathogen involved in the immune response, expressed in recombinant viral vectors such as vaccinia virus or baculovirus. More recently, recombinant DNA techniques have been utilized to produce herpes virus deletion mutants or polioviruses that mimic attenuated viruses found in nature or known host range mutants. Until 1990, the negative strand RNA viruses were not amenable to site-specific manipulation at all, and thus could not be genetically engineered.

The yield of attenuated live influenza viruses produced can be adversely affected by the immune responses, e.g., the interferon response, in the host in which they replicate. Additionally, the infected cells within the egg can become apoptotic before viral yield is maximized. Thus, although these viruses are immunogenic and non-pathogenic, they are often difficult to propagate in conventional egg substrates for the purposes of making vaccines. Hence, some embodiments of the present invention provide for means and methods using RNA effector molecules to modulate the expression of adverse host cell responses in the egg and therefore increase viral yield. For example, some embodiments of the present invention relate to injecting an RNA effector molecule (e.g., a dsRNA) into the egg, e.g., into the amniotic space, or the chorioallantoic membrane (CAM), prior to, during or after the viral or vector inoculation to inhibit cellular and anti-viral processes that compromise the yield and quality of the viral/immunogenic product harvest. The compositions and methods described herein can also be used in a cell culture-based method using cells often used for biologics production, for example, chicken fibroblasts.

The present invention provides for enhancing production of a viral product by introducing into the egg a RNA effector molecule to modulate expression of a target gene, optionally encoding a protein, that affects cell growth, cell division, cell viability, apoptosis, the immune response of the cells, nutrient handling, and/or other properties related to cell growth and/or division within the egg. Thus, in some embodiments, the production of a viral product in an embryonated egg is enhanced by introducing into the egg a RNA effector molecule that modulates expression of a viral protein such that the infectivity and/or load of the virus in the host cell is increased. In additional embodiments, production of a viral product in an embryonated egg is enhanced by introducing a RNA effector molecule that modulates expression of a host (egg) cell protein involved in viral infection or reproduction such that the infectivity and/or load of the virus is increased. In further embodiments, production is enhanced by introducing into the egg a RNA effector molecule that transiently inhibits expression of viral proteins during the growth phase.

The modulation of expression (e.g., inhibition) of a target gene by a RNA effector molecule can be further alleviated by introducing a second RNA effector molecule, wherein at least a portion of the second RNA effector molecule is complementary to a target gene encoding a protein that mediates RNAi in the host cell. For example, the modulation of expression of a target gene can be alleviated by introducing into the egg a RNA effector molecule that inhibits expression of an argonaute protein (e.g., argonaute-2) or other component of the RNAi pathway of the cell. In one embodiment, the biological product is a virus and expression of the virus is transiently inhibited by contacting the cell with a first RNA effector molecule targeted to a viral protein. The inhibition of expression of the viral product is then alleviated by introducing into the egg a second RNA effector molecule targeted against a gene encoding a protein of the RNAi pathway.

Additionally, the production of virus can be enhanced by introducing into the egg a RNA effector molecule during the production phase to modulate expression of a target gene encoding a protein that affects protein expression, post-translational modification, folding, secretion, and/or other processes related to production and/or recovery of the virus. Alternatively, the production of a viral product is enhanced by introducing into the egg a RNA effector molecule which inhibits cell growth and/or cell division during the viral production phase.

In some embodiments, the enhancement of production of a viral product, upon modulation of a target gene, is detected by monitoring one or more measurable bioprocess parameters, such as cell density, medium pH, oxygen levels, glucose levels, lactic acid levels, temperature, viral protein, or viral particle production. Viral protein production can be measured as specific productivity (SP) (the concentration of a product in solution) and can be expressed as mg/L or g/L; in the alternative, specific productivity can be expressed as pg/cell/day. An increase in SP can refer to an absolute or relative increase in the concentration of a protein product produced under two defined set of conditions. Alternatively, virus can be titered by well known plaque assays, measured as plaque forming units per mL (PFU/mL).

### II. ENHANCING BIOPROCESSING

The invention provides methods for enhancing the egg-based production of biological products, such as immunogenic agents, using the RNA effector molecules described herein. The methods generally comprise contacting a cell in the egg with a RNA effector molecule, a portion of which is complementary to a target gene, and maintaining the cell in egg culture for a time sufficient to modulate expression of the target gene, wherein the modulation enhances production of the immunogenic agent from the egg, and isolating the immunogenic agent from the egg. The RNA effector molecules can be added to the egg under conditions that permit production of a biological product, e.g., to provide transient modulation of the target gene thereby enhancing expression of the biological product.

In one embodiment, the production of an immunogenic agent is enhanced by contacting the egg cells with a RNA effector molecule provided herein during the production phase to modulate expression of a target gene encoding a protein that affects protein expression, post-translational modification, folding, secretion, and/or other processes related to production and/or recovery of the immunogenic agent. In further embodiments, the production of an immunogenic agent is enhanced by contacting egg cells with a RNA effector molecule that inhibits cell growth and/or cell division during the production phase.

In some embodiments, the production of an immunogenic agent in an egg is enhanced by contacting the egg with a RNA effector molecule which modulates expression of a protein of a contaminating virus, thus reducing the contaminant's infectivity and/or viral load in the host cell. In additional embodiments, production of an immunogenic agent in an embryonated egg host cell is enhanced by contacting the cell with a RNA effector molecule which modulates expression of a host cell protein involved in viral infection, e.g., a cell membrane ligand, or viral reproduction, thus reducing the infectivity and/or load of contaminating viruses in the host cell.

In some embodiments, the enhancement of production of a biological product upon modulation of a target gene is detected by monitoring one or more measurable bioprocess parameters, such as a parameter selected from the group consisting of: cell density, pH, oxygen levels, glucose levels, lactic acid levels, temperature, and protein production. Protein production can be measured as specific productivity (SP) (the concentration of a product, such as a heterologously expressed polypeptide, in solution) and can be expressed as mg/mL or g/mL; in the alternative, specific productivity can be expressed as mg/egg/day. An increase in SP can refer to an absolute or relative increase in the concentration of a product produced under two defined set of conditions (e.g., when compared with controls not treated with RNA effector molecule(s)). For example, in influenza production, enhancement can be monitored by measuring the amount of the viral NP protein in a sample.

In some embodiments, RNA effector compositions include two or more RNA effector molecules, e.g., comprise two, three, four or more RNA effector molecules. In various embodiments, the two or more RNA effector molecules are capable of modulating expression of the same target gene and/or one or more additional target genes. Advantageously, certain compositions comprising multiple RNA effector molecules are more effective in enhancing production of an immunogenic agent, or one or more aspects of such production, than separate compositions comprising the individual RNA effector molecules.

In other embodiments, a plurality of different RNA effector molecules are contacted with the egg cells and permit modulation of one or more target genes. In one embodiment, at least one of the plurality of different RNA effector molecules is a RNA effector molecule that modulates expression of glutaminase, glutamine dehydrogenase, or LDH. In another embodiment, RNA effector molecules targeting Bax and Bak are co-administered to an egg during production of the immunogenic agent and can optionally contain at least one additional RNA effector molecule or agent. In another embodiment, a plurality of different RNA effector molecules is contacted with the cells in the egg to permit modulation of Bax, Bak, and LDH expression. In another embodiment, a plurality of different RNA effector molecules is contacted with the cells in the egg to permit modulation of expression of Bax and Bak, as well as glutaminase and/or glutamine dehydrogenase.

When a plurality of different RNA effector molecules are used to modulate expression of one or more target genes the plurality of RNA effector molecules can be contacted with egg cells simultaneously or separately. In addition, each RNA effector molecule can have its own dosage regimen. For example, one can prepare a composition comprising a plurality of RNA effector molecules are contacted with a cell. Alternatively, one can administer one RNA effector molecule at a time to the egg. In this manner, one can easily tailor the average percent inhibition desired for each target gene by altering the frequency of administration of a particular RNA effector molecule. For example, strong inhibition (e.g., >80% inhibition) of lactate dehydrogenase (LDH) can not always be necessary to significantly improve production of a biological product and under some conditions it may be preferable to have some residual LDH activity. Thus, one may desire to contact a cell with an RNA effector molecule targeting LDH at a lower frequency (e.g., less often) or at a lower dosage (e.g., lower multiples over the IC₅₀) than the dosage for other RNA effector molecules. Contacting a cell with each RNA effector molecule separately can also prevent interactions between RNA effector molecules that can reduce efficiency of target gene modulation. For ease of use and to prevent potential contamination it may be preferred to administer a cocktail of different RNA effector molecules, thereby reducing the number of doses required and minimizing the chance of introducing a contaminant to the egg.

In yet further embodiments, the modulation of expression (e.g., inhibition) of a target gene by a RNA effector molecule can be alleviated by contacting the cell with second RNA effector molecule, wherein at least a portion of the second RNA effector molecule is complementary to a target gene encoding a protein that mediates RNAi in the host cell. For example, the modulation of expression of a target gene can be alleviated by contacting the cell with a RNA effector molecule that inhibits expression of an argonaute protein (e.g., Argonaute-2) or other component of the RNAi pathway of the cell. In one embodiment, the immunogenic agent is a recombinant protein and expression of the product is transiently inhibited by contacting the egg cell with a first RNA effector molecule targeted to the transgene encoding the immunogenic agent. The inhibition of expression of the immunogenic agent is then alleviated by contacting the egg cell with a second RNA effector molecule targeted against a gene encoding a protein of the cellular RNAi pathway.

In further embodiments, the methods further comprise administering to the embryonated egg with a second agent. The second agent can be an immunosuppressive agent; a growth factor; an apoptosis inhibitor; a kinase inhibitor; a phosphatase inhibitor; a protease inhibitor; an inhibitor of pathogens (e.g., where a virus is the biological product, an agent that inhibits growth and/or propagation of endogenous or contaminating viruses, or fungal or bacterial pathogens); or a histone demethylating agent.

Production of a viral product can be enhanced by reducing the expression of a protein that binds to the product. For example, in producing a viral protein, it can be advantageous to reduce or inhibit expression of its receptor/ligand so that its production in the cell does not elicit a biological response. As another example, in producing a growth factor, a hormone or a cell signaling protein, it can be advantageous to reduce or inhibit expression of its receptor/ligand so that its production in the host cell does not elicit a biological response by the cell. It is known to a skilled artisan that a receptor can be a cell surface receptor or an internal (e.g., nuclear) receptor. Therefore, in one example, production of a biological product such as influenza virus, can be enhanced by modulating (e.g., reducing) the level of the receptor present in the cell (e.g., sialic acid). The expression of the binding partner can be modulated by contacting the host cell with an RNA effector molecule directed at the genes in the receptor pathway according to methods described herein.

Proteins expressed in eukaryotic cells can undergo several post-translational modifications that can impair viral protein production and/or the structure, biological activity, stability, homogeneity, and/or other properties of viral particles. Many of these modifications occur spontaneously during cell growth and polypeptide expression and can occur at several sites, including the peptide backbone, the amino acid side-chains, and the amino and/or carboxyl termini of a given polypeptide. In addition, a given polypeptide can comprise several different types of modifications. For example, proteins expressed in avian cells can be subject to acetylation, acylation, ADP-ribosylation, amidation, ubiquitination, methionine oxidation, disulfide bond formation, methylation, demethylation, sulfation, formation of cysteine, formation of pyroglutamate, formylation, gamma-carboxylation, hydroxylation, iodination, myristoylation, oxidation, proteolytic processing, phosphorylation, prenylation, racemization, glycosylation, gluconoylation, sequence mutations, N-terminal glutamine cyclization and deamidation, and asparagine deamidation.

Post-translational modifications can require additional bioprocess steps to separate modified and unmodified polypeptides, increasing costs and reducing efficiency of virus production. Accordingly, in some embodiments, production of a viral polypeptide a cell is enhanced by modulating the expression of a target gene encoding a protein that affects post-translational modification. In additional embodiments, viral protein production is enhanced by modulating the expression of a first target gene encoding a protein that affects a first post-translational modification and modulating the expression of a second target gene encoding a protein that affects a second post-translational modification.

The viral product, particularly, the viral surface membrane proteins, comprise a glycoprotein, and viral production is enhanced by modulating expression of a target gene which encodes a protein involved in protein glycosylation. Glycosylation patterns are often important determinants of the structure and function of mammalian glycoproteins, and can influence the solubility, thermal stability, protease resistance, antigenicity, immunogenicity, serum half-life, stability, and biological activity of glycoproteins.

In some instances, the rate of protein production and the yield of recovered protein is directly related to the rate of protein folding and secretion by the host cells. For example, an accumulation of misfolded proteins in the endoplasmic reticulum (ER) of host cells can slow or stop secretion via the unfolded protein response (UPR) pathway. The UPR is triggered by stress-sensing proteins in the ER membrane which detect excess unfolded proteins. UPR activation leads to the upregulation of chaperone proteins (e.g., Bip) which bind to misfolded proteins and facilitate proper folding. UPR activation also upregulates the transcription factors XBP-1 and CHOP. CHOP generally functions as a negative regulator of cell growth, differentiation and survival, and its upregulation via the UPR causes cell cycle arrest and increases the rate of protein folding and secretion to clear excess unfolded proteins from the cell. Hence, cell cycle can be promoted initially, then repressed during virus production phase to increase viral product yield. An increase the rate of protein secretion by the host cells can be measured by, e.g., monitoring the amount of protein present in the extracellular milieu over time.

In some embodiments of the present invention, it is advantageous to temporarily inhibit viral replication, for example, until RNAi has inhibited the host cell immune response, such that viral replication ensues after adequate suppression of the cell immune response. Rather than initiate RNAi prior to viral inoculation in a two-step fashion, this embodiment introduces the RNA effector molecule(s) with the viral inoculum, avoiding extra interruption (and possible contamination) of the bioprocess. This embodiment provides an approach for enhancing viral load and the yield of immunogenic agent.

For example, siRNAs specific for conserved regions of the viral genome can inhibit influenza virus production in both cell lines and embryonated chicken eggs. The inhibition depends on the presence of a functional antisense strand in the siRNA duplex, suggesting that viral mRNA is the target of RNA interference. siRNA specific for nucleocapsid (NP) or a component of the RNA transcriptase (PA) abolishes the accumulation of the corresponding mRNA, virion RNA, its complementary RNA, and broadly inhibited the accumulation of other viral, but not host cell, RNAs. RNA effector molecules useful for inhibiting influenza A, e.g., the PB, PA, NP, M, and NS genes, are reported in Ge et al., 100 PNAS 2718-23 (2003). siRNAs, some of which are modified, useful for inhibiting expression of influenza A NP and PA genes are reported in WO 2007/056861.

Influenza A virus has an eight-segmented RNA genome. Three of the eight RNA segments encode three components of the RNA transcriptase (PA, PB1, and PB2). Three additional RNA segments encode the major glycoproteins: hemagglutinin (HA), neuraminidase (NA), and nucleocapsid protein (NP). Each of the remaining two RNA segments encodes two proteins, either M1 or M2, and NS1 or NS2, which function either as viral structural proteins or in the viral life cycle. There are 15 HA subtypes and 9 NA subtypes known among influenza A viruses. Because extensive differences in nucleotide sequences are present in genes among virus isolates from humans and different species, siRNAs that remain effective despite antigenic drifts and shifts can be designed by focusing on the viral genes that are conserved among different subtypes and strains of virus from human and non-human species. Additionally, the design should avoid identity with host cell genes. There are influenza sequence databases available on the internet. The amount of RNA effector molecule targeting viral replication can be balanced against the impact on viral replication. For example, because the NP-1496 inhibits viral replication 200- to 30.000-fold, depending on the multiplicity of infection, these factors should be considered during the formulation of RNA effector composition(s). Of the siRNAs designed by Ge et al., NP-1496, PA-2087 and PB 1-2257 that potently inhibited influenza virus production in both MDCK cells and in chicken embryos, whereas siRNAs NP-231, M-37 and PB1-129 were less effective in MDCK cells, and ineffective in chicken embryos.

Alternatively, the stability of the RNA effector molecule can be manipulated to adjust its half-life within the host cell. For example, numerous nucleotide modifications are envisioned herein that effect the half-life of the RNA effector molecule. When a siRNA is designed to target a viral gene, it can be prepared in a form that makes it labile (unmodified), whereas the RNA effector molecule(s) targeting the host cell immune response genes are modified for added stability.

In another aspect of this embodiment, virus is encapsulated in a control release vehicle, such that although it is administered to the egg concurrent with the RNA effector molecules, viral infection is delayed a period of time sufficient for RNAi inhibition of cell immune response. Approaches to injectable controlled release viral delivery include many approaches involving injectable polymers, polyelectrolytes, polymer microspheres, and polymer-virus conjugates. Wang & Pham, 5 Exp. Op. Drug. Deliv. 385-401 (2008).

In an alternative approach, cells are inoculated with virus, unbound virus is washed from the cells, and these infected cells are then introduced to the embryonated egg concurrent with the RNA effector molecule. Cells have been used as vehicles to carry and produce viral vectors in vivo. See Sonabend et al., 11 Gene Ther. Mol. Biol. 79-92 (2007). Because viral replication takes several hours (e.g., about 4 hours for influenza viral shed from MDCK cells), this serves as a control release vehicle for virus into the embryonated egg.

Alternatively, the multiplicity of infection can be relatively low compared to the RNA effector molecules and the cell culture density, thus allowing greater influence of the RNA effector in the cell population as the viral titer builds.

Host Cell Immune Response

In some embodiments, production of an immunogenic agent in an embryonated egg is further enhanced by introducing a RNA effector molecule that modulates expression of a cell protein involved in microbial infection or reproduction such that the infectivity and/or load of the desired microbe is increased. Modulating the egg's immune response can also be beneficial in the production of certain immunogenic agents that are themselves involved in modulating the immune response (e.g., influenza and the like).

Several human, mammalian and avian viruses can be cultivated in embryonated eggs for either virus production (e.g., ultimately for vaccine production) or heterologous protein expression. Infection or transfection results in the accumulation of an immunogenic agent, such as recombinant antigens or live virus particles, that can be collected from the egg after a suitable incubation period. For example, the standard method of vaccine production consists of culturing eggs; infecting with a live virus (e.g., influenza); incubation; harvesting of egg tissues; downstream processing; and filling and finishing. For the classic inactivated influenza vaccine, purification, inactivation, and stabilization of this harvested immunogenic agent yields vaccine product, which techniques are well known in the art.

Recombinant DNA technology and genetic engineering techniques, in theory, can afford a superior approach to producing an attenuated virus because specific mutations are deliberately engineered into the viral genome. The genetic alterations required for attenuation of viruses are not always predictable, however. In general, the attempts to use recombinant DNA technology to engineer viral vaccines have been directed to the production of subunit vaccines which contain only the protein subunits of the pathogen involved in the immune response, expressed in recombinant viral vectors such as vaccinia virus or baculovirus. More recently, recombinant DNA techniques have been utilized to produce herpes virus deletion mutants or polioviruses that mimic attenuated viruses found in nature or known host range mutants.

The yield of an immunogenic agent, such as an attenuated live influenza virus or an immunomodulatory polypeptide, made in an egg can be adversely affected by the immune response of the host cell, e.g., the interferon response of the host cell in which the virus or viral vector is replicated. Additionally, the infected host cell(s) can become apoptotic before viral yield is maximized. Thus, although these attenuated viruses are immunogenic and non-pathogenic, they are often difficult to propagate in conventional cell substrates for the purposes of making vaccines. Hence, some embodiments of the present invention provide for compositions and methods using a RNA effector molecules to modulate the expression of adverse host cell responses and therefore increase yield. For example, some embodiments of the present invention relate to contacting an egg cell with a RNAi-based product (e.g., siRNA) prior to, during or after the viral infection or vector administration, to inhibit cellular and anti-viral processes that compromise the yield and quality of the product harvest.

The use of egg-based bioprocesses for the manufacture of viral product is enhanced, in some embodiments, by modulating expression of a target gene that affecting the host cell's reaction to viral infection. This approach is useful where the biological product is viral or otherwise immunomodulatory, or where viral vectors are used to introduce heterologous proteins into the host cell.

For example, in some embodiments the target gene is a cell interferon protein or a protein associated with interferon signaling. In particular, the gene can be an interferon gene such as IFN-α (GeneID: 396398,); IFN-β (GeneID: 554219, modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3156155-315633 (sense) and SEQ ID NOs:3156181-3156206 (antisense)); or IFN-γ, (IFN-γ GeneID: 396054). The gene can be an interferon receptor such as IFNAR1 (interferon α, β and ω receptor 1, GeneID: 395665, the expression of which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3514605-3154633 (sense) and SEQ ID NOs:3154634-3154662 (antisense)), IFNAR2 (interferon α, β and ω receptor 2) (GeneID: 395664), IFNGR1 (interferon-γ receptor 1) (GeneID: 421685) or IFNGR2 (interferon-γ receptor 2 [interferon γ transducer 1]) (GeneID: 418502).

In some embodiments, the gene can be associated with interferon signaling such as STAT-1 (signal transducer and activator of transcription 1, GeneID: 424044), STAT-2, STAT-3 (GeneID:420027), STAT-4 (GeneID: 768406), STAT-5 (GeneID: 395556; JAK-1 (Janus kinase 1) (Jak1, GeneID: 395681; JAK-2 (Jak2, GeneID: 374199), JAK-3 (Jak3, GeneID: 395845), IRF1 (interferon regulatory factor 1) (GeneID: 396384), IRF2 (GeneID: 396115), IRF3 (GeneID: 396330) the expression of which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3288948-3289249 (sense) and SEQ ID NOs:3289250-3289551 (antisense)), IRF4 (GeneID: 374179), IRF5 (GeneID: 430409), IRF6 (GeneID: 419863), IRF7 (GeneID: 396330), IRF8 (GeneID:396385), IRF 9 (e.g., Danio rerio irf9, GeneID: 403013), or IRF10 (GeneID: 395243).

Similarly, the target gene can encode an interferon-induced protein such as 2′,5′ oligoadenylate synthetases (2-50AS), an interferon induced antiviral protein; RNaseL (ribonuclease L (2′,5′-oligoisoadenylate synthetase-dependent), GeneID: 424410 (Silverman et al., 14 J. Interferon Res. 101-04 (1994)); IFITM1, IFITM2 and IFITM3 (Brass et al., 139 Cell 1243-54 (2009)); Proinflammatory cytokines; MYD88 (myeloid differentiation primary response gene) up-regulated upon viral challenge, GeneID: 420420); TRIF (toll-like receptor adaptor molecule 1, GeneID: 100008585) (Hghighi et al., Clin. Vacc. Immunol. (Jan. 13, 2010)); Mx (MX1 myxovirus (influenza virus) resistance 1, interferon-inducible protein p78) (mxl, GeneID: 395313; Haller et al., 9 Microbes Infect. 1636-43 (2007); or dsRNA-dependent protein kinase (PKR) (eukaryotic translation initiation factor 2-α kinase 2 (EIF2AK2, Li et al., 106 PNAS 16410-05 (2009).

Hence, for example, the target gene Mx1 can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NO:3286682-3286975 (sense) and SEQ ID NO:3286976-3287269 (antisense)).

The target gene PKR (EIF2AK2) (Li et al., 106 PNAS 16410-05 (2009)), can be modulated by use of a corresponding RNA effector molecule comprising a sense strand and an antisense strand wherein one strand comprises at least 16 contiguous nucleotides (e.g., at least 17, at least 18, at least 19 nucleotides) of the oligonucleotides shown in the following Table 1:

TABLE 1
Example sRNA targeting Gallus gallus EIF2AK2

| Sense | Antisense |
|---|---|
| CCACUGAGUGAUUCAGCCU | AGGCUGAAUCACUCAGUGG |
| GGUACAGGCGUUGGUAAGA | UCUUACCAACGCCUGUACC |
| CAGGCGUUGGUAAGAGUAA | UUACUCUUACCAACGCCUG |
| GAAUGUGCAUACUUCGGAU | AUCCGAAGUAUGCACAUUC |
| CAUACUUCGGAUGUAGUGA | UCACUACAUCCGAAGUAUG |
| GACAUUGCAGCUAGUUGAU | AUCAACUAGCUGCAAUGUC |
| CAUUGCAGCUAGUUGAUUA | UAAUCAACUAGCUGCAAUG |
| CCACGCUCCAAUGUAUUCU | AGAAUACAUUGGAGCGUGG |
| GUAAUUAGUGGUCAUGUAU | AUACAUGACCACUAAUUAC |
| CAUGAACUCAGUAAUUCCU | AGGAAUUACUGAGUUCAUG |
| GAGUCAUGGGGUAUUACCU | AGGUAAUACCCCAUGACUC |
| GGUAUUACCUUUAAAGACU | AGUCUUUAAAGGUAAUACC |
| GAAAGACAUGUCCCUAUCU | AGAUAGGGACAUGUCUUUC |
| GAGCCUUCAAAUUGUCGGA | UCCGACAAUUUGAAGGCUC |
| GAGUAUUGGCACCUAAUUU | AAAUUAGGUGCCAAUACUC |
| GGUUUCGUCAGCAGUAUAA | UUAUACUGCUGACGAAACC |
| CUAUGCAAUCAAACGAGUU | AACUCGUUUGAUUGCAUAG |
| GUUAAUAAAUAGGAACGUA | UACGUUCCUAUUUAUUAAC |
| GCUCGCGAAUCUUGAACAU | AUGUUCAAGAUUCGCGAGC |
| CGCGAAUCUUGAACAUGAA | UUCAUGUUCAAGAUUCGCG |
| GAAUUCUAUCGUAGCUGUU | AACAGCUACGAUAGAAUUC |
| GAAUAUAUUCCUAUCAUAU | AUAUGAUAGGAAUAUAUUC |
| CUUUGGUCUCGUGACUUCU | AGAAGUCACGAGACCAAAG |
| CCCUCUGACUAAGAACCGA | UCGGUUCUUAGUCAGAGGG |
| GAGGAACACAGUCAUAUAU | AUAUAUGACUGUGUUCCUC |
| GAUAUGGAAAGGAAGUAGA | UCUACUUCCUUUCCAUAUC |
| GGUAUGGCAGGAUGUUAGA | UCUAACAUCCUGCCAUACC |
| CCAGGUACCCAUAAUCAAA | UUUGAUUAUGGGUACCUGG |
| GACAACUCGCAUAAAGCUU | AAGCUUUAUGCGAGUUGUC |
| CACUUCUUUUAGGUGAACU | AGUUCACCUAAAAGAAGUG |
| CCUUAAGUAUUUAGCUUUU | AAAAGCUAAAUACUUAAGG |
| GUUCUUCCUUAUAGGAACA | UGUUCCUAUAAGGAAGAAC |
| CAGGUAGGGUCCUCUUAAU | AUUAAGAGGACCCUACCUG |
| GUAGGGUCCUCUUAAUACA | UGUAUUAAGAGGACCCUAC |
| CUCCUAUACAGUACGGUUU | AAACCGUACUGUAUAGGAG |
| CUAUACAGUACGGUUUUAA | UUAAAACCGUACUGUAUAG |
| GUACGGUUUUAAUCGCCUA | UAGGCGAUUAAAACCGUAC |
| GGUUUUAAUCGCCUAUUAU | AUAAUAGGCGAUUAAAACC |
| GAUUAUAGGUGUACCUGAA | UUCAGGUACACCUAUAAUC |
| GUCAGCUCAACAUAAGGUA | UACCUUAUGUUGAGCUGAC |
| CUGAUUGACCGUUACUCUU | AAGAGUAACGGUCAAUCAG |
| GACCGUUACUCUUUGGUUA | UAACCAAAGAGUAACGGUC |
| CGUUACUCUUUGGUUAUAU | AUAUAACCAAAGAGUAACG |
| GGUUAUAUACUUAAGAGAU | AUCUCUUAAGUAUAUAACC |
| CUUAAGAGAUUUCUCGUUU | AAACGAGAAAUCUCUUAAG |
| GAUUUCUCGUUUGACUAAA | UUUAGUCAAACGAGAAAUC |
| CUCGUUUGACUAAAUAAGA | UCUUAUUUAGUCAAACGAG |

In another embodiment, the biologic is produced by an egg wherein the cells have been transfected with one or more retroviral vectors. For example, upon transfection with a first retroviral vector, expression of the retroviral vector Env and/or Gag molecule is transiently inhibited by contacting the cell with a first RNA effector molecule (i.e., targeting the env gene or gag gene), allowing more efficient transfection with a second retroviral vector. For example, a first retroviral vector encodes a first peptide and a second retroviral vector encodes a second, complementary peptide (such that the biological product contains both peptides). Additionally, the inhibition of expression can be alleviated by introducing into the cell an additionally RNA effector molecule targeted against a gene encoding a protein of the RNAi pathway.

In some embodiments, the target gene is a regulatory element or gene of an endogenous avian retrovirus (EAV). For example, in particular embodiments the target gene can encode an avian leukosis virus LTR, env protein, or gag protein. See Tsang et al., 73 J. Virol. 5843-51 (1999).

In additional embodiments, the target gene is a cell protein that mediates viral infectivity, such as TLR3 that detects dsRNA (GeneID: 422720), that can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3155965-3156011 (sense) and SEQ ID NOs:3156012-3156058 (antisense); TLR7 that detects ssRNA (GeneID: 418638); TLR21, that recognizes unmethylated DNA with CpG motifs (Tlr3, GeneID: 415623); RIG-1 involved with viral sensing (Myong et al., 323 Science 1070-74 (2009)); LPGP2 and other RIG-1-like receptors, which are positive regulators of viral sensing (Satoh et al., 107 PNAS 1261-62 (2010); Nakhaei et al., 2009); TRIM25 (GeneID: 417401; Gack et al., 5 Cell Host Microb. 439-49 (2009)); or MAVSNISA/IPS-1/Gardif, which interacts with RIG-1 to initiate an antiviral signaling cascade (Cui et al., 29 Mol. Cell. 169-79 (2008)); Kawai et al., 6 Nat. Immunol. 981-88 (2005)). Thus, for example, a RNA effector molecule that targets MAVS can comprise a sense strand and an antisense strand wherein one strand comprises at least 16 contiguous nucleotides (e.g., at least 17, at least 18, at least 19 nucleotides) of the nucleotides selected from SEQ ID NOs:3156207-3156253 (sense) and SEQ ID NOs:3156254-3156300 (antisense).

A composition, in alternative embodiments, can comprise one or more RNA effector molecules capable of modulating expression of one or multiple genes relating to a common biological process or property of the cell, for example the interferon signaling pathway including IFN, STAT proteins or other proteins in the JAK-STAT signaling pathway, IFNRA1 and/or IFNRA2. For example, viral infection results in swift innate response in infected cells against potential lytic infection, transformation and/or apoptosis, which is characterized by the production of IFNα and IFNβ This signaling results in activation of IFN-stimulated genes (ISGs) that mediate the effects of IFN. IFN regulatory factor (IRFs) are family of nine cellular factors that bind to consensus IFN-stimulated response elements (ISREs) and induce other ISGs. See Kirshner et al., 79 J. Virol. 9320-24 (2005). The IFNs increase the expression of intrinsic proteins including TRIM5α, Fv, Mx, eIF2α and 2′-5′ OAS, and induce apoptosis of virus-infected cells and cellular resistance to viral infection. Koyam et al., 43 Cytokine 336-41 (2008). Hence, a particular embodiment provides for a RNA effector molecule that targets a IFNR1 gene. Other embodiments target one or more genes in the IFN signaling pathway.

Inhibition of IFN signaling responses can be determined by measuring the phosphorylated state of components of the IFN pathway following viral infection, e.g., chicken IRF3, which is phosphorylated in response to viral dsRNA. In response to type I IFN, Jak1 kinase and TyK2 kinase, subunits of the IFN receptor, STAT1, and STAT2 are rapidly tyrosine phosphorylated. Thus, in order to determine whether the RNA effector molecule inhibits IFN responses, cells can be contacted with the RNA effector molecule, and following viral infection, the cells are lysed. IFN pathway components, such as Jak1 kinase or TyK2 kinase, are immunoprecipitated from the infected cell lysates, using specific polyclonal sera or antibodies, and the tyrosine phosphorylated state of the kinase determined by immunoblot assays with an anti-phosphotyrosine antibody. See, e.g., Krishnan et al., 247 Eur. J. Biochem. 298-305 (1997). A decreased phosphorylated state of any of the components of the IFN pathway following infection with the virus indicates decreased IFN responses by the virus in response to the RNA effector molecule(s).

Efficacy of IFN signaling inhibition can also be determined by measuring the ability to bind specific DNA sequences or the translocation of transcription factors induced in response to viral infection, and RNA effector molecule treatment, e.g., targeting IRF7, STAT1, STAT2, etc. In particular, STAT 1 and STAT2 are phosphorylated and translocated from the cytoplasm to the nucleus in response to type I IFN. The ability to bind specific DNA sequences or the translocation of transcription factors can be measured by techniques known to skilled artisan, e.g., electromobility gel shift assays, cell staining, etc. Another approach to measuring inhibition of IFN-induction determines whether an extract from the egg producing the desired viral product and contacted with a RNA effector molecule is capable of conferring protective activity against viral infection. More specifically, for example, eggs are infected with the desired virus and contacted with a RNA effector. Approximately 15 to 20 hr post-infection, the eggs are harvested and assayed for viral titer, or by quantitative product-enhanced reverse transcriptase (PERT) assay, immune assays, or in vivo challenge.

Another example of an embyronated egg cell gene for which inhibition increases is protein kinase CK2 β subunit (CSKN2B). More specifically, cells in which the CSKN2B gene is dilenced exhibit increased influenza protein production, replication, and viral titer. Marjuki et al., 3 J. Mol. Signaling. 13 (2008). Hence, in some embodiments, expression of CSKN2B can be modulated by use of a corresponding RNA effector molecule comprising a sense strand and an antisense strand wherein on strand comprises at least 16 contiguous nucleotides (e.g., at least 17, at least 18, at least 19 nucleotides) selected from the nucleotides in SEQ ID NOs:3239552-3289846 (sense) and SEQ ID NOs:3289847-3290141 (antisense).

Host Cell Receptors

In some embodiments, the target gene is a host cell gene (endogenous)encoding or involved in the synthesis or regulation of a membrane receptor or other moiety. Modulating expression of the cell membrane can increase or decrease viral infection (e.g., by increasing or decreasing receptor expression), or can increase recovery of product that would otherwise adsorb to host cell membrane (by decreasing receptor expression).

For example, many viruses adhere to host cell-surface heparin, including PCV (Misinzo et al., 80 J. Virol. 3487-94 (2006); CMV (Compton et al., 193 Virology 834-41 (1993)); pseudorabies virus (Mettenleiter et al., 64 J. Virol. 278-86 (1990)); BHV-1 (Okazaki et al., 181 Virology 666-70 (1991)); swine vesicular disease virus (Escribano-Romero et al., 85 Gen. Virol. 653-63 (2004)); and HSV (WuDunn & Spear, 63 J. Virol. 52-58 (1989)). Additionally, enveloped viruses having infectivity associated with surface heparin binding include HIV-1 (Mondor et al., 72 J. Virol. 3623-34 (1998)); AAV-2 (Summerford & Samulski, 72 J. Virol. 1438-45 (1998)); equine arteritis virus (Asagoe et al., 59 J. Vet. Med. Sci. 727-28 (1997)); Venezuelan equine encephalitis virus (Bernard et al., 276 Virology 93-103 (2000)); Sindbis virus (Byrnes & Griffin, 72 J. Virol. 7349-56 (1998); Chung et al., 72 J. Virol. 1577-85 (1998)); swine fever virus (Hulst et al., 75 J. Virol. 9585-95 (2001)); porcine reproductive and respiratory syndrome virus (Jusa et al., 62 Res. Vet. Sci. 261-64 (1997)); and RSV (Krusat & Streckert, 142 Arch. Virol. 1247-54 (1997)). A number of non-enveloped virus associate with cell surface heparin as well. Some picornaviridae family members associate with cell-surface heparin, including, foot-and-mouth disease virus (FMDV) (binds in in vitro culture) (Fry et al., 18 EMBO J. 543-54 (1999); Jackson et al., 70 J. Virol. 5282-87 (1996)); coxsackie virus B3 (CVB3) (Zautner et al., 77 J. Virol. 10071-77 (2003)); Theiler's murine encephalomyelitis virus (Reddi & Lipton, 76 J. Virol. 8400-07 (2002)); and certain echovirus serotypes (Goodfellow et al., 75 J. Virol. 4918-21 (2001)).

Hence, in particular embodiments of the present invention, cellular expression of heparin can be modulated in order to decrease or increase viral adsorption to the host cell. For example, one or more RNA effector molecule(s) can target one or more genes associated with heparin synthesis or structure, such as epimerases, xylosyltransferases, galactosyltransferases, N-acetylglucosaminyl transferases, glucuronosyltransferases, or 2-O-sulfotransferases. See, e.g., Rostand & Esko, 65 Infect. Immun 1-8 (1997).

In the instance where the expression of cell-surface heparin sought to be increased, a RNA effector molecule can target genes associated with heparin degradation, such as genes encoding heparanase (hep) (hep 1, GeneID: 373981; hep 2, GeneID: 423834). Gingis-Velitski et al., 279 J. Biol. Chem. 44084-92 (2004). Similarly, the infectivity of influenza virus is dependent on the presence of sialic acid on the cell surface (Pedroso et al., 1236 Biochim. Biophys. Acta 323-30 (1995), as is the infectivity of rotaviruses (Is a et al., 23 Glycoconjugate J. 27-37 (2006); Fukudome et al., 172 Virol. 196-205 (1989)), other reoviruses (Paul et al., 172 Virol. 382-85 (1989)), and bovine coronaviruses (Schulze & Herrler, 73 J. Gen. Virol. 901-06 (1992)).

Thus, in some embodiments the gene target(s) include those involved in host sialidase in avian cells (see Wang et al., 10 BMC Genomics 512 (2009)). Because influenza binds to cell surface sialic acid residues, decreased sialidase can increase the rate of purification. Target genes include, for example, NEU2 sialidase 2 (cytosolic sialidase) (Neu2, GeneID: 430542); NEU3 sialidase 3 (membrane sialidase) (Neu3, GeneID: 68823); solute carrier family 35 (CMP-sialic acid transporter) member A1 (Slc35A1, that can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154345-3154368 (sense) and SEQ ID NOs:3154369-3154392 (antisense)); UDP-N-acetylglucosamine 2-epimerase/N-acetylmannosamine kinase (Gne, modulated by use of a corresponding RNA effector molecule comprising an antisense strand comprising at least 16 contiguous nucleotides (e.g., at least 17, at least 18, at least 19 nucleotides) of the nucleotides in SEQ ID NOs:3154297-3154320 (sense) and SEQ ID NOs:3154321-3154344 (antisense)); cytidine monophospho-N-acetylneuraminic acid synthetase (Cmas), the expression of which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154249-3154272 (sense) and SEQ ID NOs:3154273-3154296 (antisense)); UDP-Gal:βGlcNAc β1,4-galactosyltransferase (B4GalT1), for which can be expression modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154153-3154176 (sense) and SEQ ID NOs:3154177-3154200 (antisense); and UDP-Gal:βGlcNAc β1-1,4-galactosyltransferase, polypeptide 6 (B4GalT6), for which expression can modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154201-3154224 (sense) and SEQ ID NOs: 3154225-3154248 (antisense).

Sialic acid residues on host cell-surface glycoproteins are receptors for influenza virus. Influenza A and B bind to the most abundant sialic acid, N-acetylneuraminic acid, while influenza C binds to 9-O-acetyl-N-acetylneuraminic acid, for adsorption. The binding of influenza A is mediated by hemagglutinin. More specifically, hemagglutinin, a viral glycoprotein, functions in the binding of the virus to cells via the recognition of sialic acid residues on the cell, and this binding initializes the association of the virus with the cells. Hemagglutinin is the major virulence (disease-causing) factor of the influenza virus. Hemagglutinin is also responsible for subsequent fusion of viral and host membranes in the intracellular endosome (i.e., after the virus has been taken up by endocytosis). After endocytosis brings the virus into the cell in an endosome, acidification of the endosome (˜pH 5.5) induces conformational changes in hemagglutinin that promote fusion with the endosomal membrane, thereby promoting the release of the flu virus into the host cytoplasm.

Neuraminidase is the common name for acetyl-neuraminyl hydrolase, a glycoprotein enzyme that removes residues called N-Acetyl-neuraminic acid from the sugar chains of other glycoproteins. The disruption of the host cell neuraminic acid residues allows the virus to both enter cells to initiate viral replication and pass out of the cells in which it is replicating: hemagglutinin binds cell-surface sialic acid, and sialic acids are then cleaved by the viral neuraminidase to promote efficient release of progeny virus particles. Possession of neuraminidase also keeps virus particles from aggregating. A typical influenza virus particle contains some 500 molecules of hemagglutinin and about 100 molecules of neuraminidase.

Inhibitors of hemagglutinin and neuraminidase and have been developed in an effort to thwart the viral infection. Some inhibitors are structurally similar to the sialic acid on the surface of cells and serve as decoys. The rationale is that the virus binds to the inhibitor rather than to the cells.

In one embodiment of the present invention, sialic acid or a derivative or analog of sialic acid, is added to the eggs and inhibits influenza viral infection until such time as a RNA effector molecule modulates a target cellular molecular function. Influenza-targeting sialic acid decoys have been used both in cell culture and embryonated eggs. Woods et al., 37 Antimicrobial Ag. Chemother. 1473-79 (1993).

Sialic acid, or one of its decoys, can be selected based on its affinity for hemagglutinin. For example, benzyl-α-NeuSAc; 2-d-2H_{eq}-Neu5Ac; methyl-α-NeuSAc; methyl-α-9-d-NeuSAc; (4-isothiocyano)benzyl-α-NeuSAc; and 2-naphthyl-α-NeuSAc are relatively potent sialic acid decoys, inhibiting influenza A hemagglutinin adsorption to erythrocytes (IC₅₀s ranged from ˜1.7 to ˜8 mM). In comparison, 2,7-d₂-2H_{eq}-Neu5Ac; 2,8-d₂-2H_{eq}-Neu5Ac, 2,7-d₂-2H_{eq}-7,8-epi₂-Neu5Ac; 2,-d-2H_{eq}-8-epi-Neu5Ac; and benzyl-α-8,9-isopropylidene-NeuSAc are relatively weak sialic acid decoys (IC₅₀s ranged from ˜17 to ˜40 mM). Kelm et al., 205 Eur. J. Biochem. 147-53 (1992).

Other molecules, such as glycoproteins, can compete with sialic acid for the lectin-like activity of influenza hemagglutination. For example, the sialoglycoprotein fetuin has been shown to compete with the lectin-binding compounds in plum that inhibit influenza A adsorption to MDCK cells in vitro. Yingsakmongkon et al., 31 Biol. Pharm. Bull. 511-15 (2008). Additionally, the collectin surfactant protein A and scavenger receptor-rich glycoprotein 340 (gp340) act like mucins in that they provide sialic acid ligands that bind to the influenza A viral hemagglutinin. White et al., 288 Am. J. Physiol. Lung Cell Mol. Physiol. L₈₃₁ (2004).

Sialic acid decoys can also be selected based on inhibition of both viral binding to hemagglutinin and neuraminidase activity. For example, an O-glycoside sialic acid derivative Neu5Ac3F-DSPE(4), in which the C-3 position is modified with an axial fluorine atom, inhibited both the binding activity of influenza virus hemagglutinin and the catalytic hydrolysis of its sialidase. The inhibitory effect of Neu5Ac3F-DSPE(4) against influenza infection of MDCK cells was examined, and it was found that the derivative inhibited influenza infection with IC₅₀ value of 5.6 μM based on cytopathic effects. Guo et al., 12 Glycobio. 183-90 (2002). Surfactant protein D binds in a calcium-dependent manner to carbohydrate attachments on the viral hemagglutinin and neuraminidase. White et al., 2004. Hence, the inhibitory effects of surfactant protein D can be regulated by addition of calcium and, optionally, subsequent addition of a chelating agent such as EDTA.

Additionally, there are viruses that combine hemagglutinin-neuraminidase into a single viral protein that has both hemagglutinin and neuraminidase activity. (This is in contrast to the proteins found in influenza, where both functions exist, but in different proteins.) These include Mumps hemagglutinin-neuraminidase and Parainfluenza hemagglutinin-neuraminidase. Human parainfluenza viruses are important respiratory tract pathogens, especially of children. Parainfluenza virus inhibitors BCX 2798 and BCX 2855 were designed based on the three-dimensional structure of the hemagglutinin-neuraminidase protein. The compounds were highly effective in inhibiting hemagglutinin and neuraminidase activities and the growth of several parainfluenza viruses in LLC-MK2 cells. The LC₅₀ ranged from 0.02 to 20.0 μM in inhibition assays. The concentrations required to inhibit virus replication to 50% of the level of the control ranged from 0.7 to 11.5 μM. Alymova et al., 48 Antimicrobial Ag. Chemo. 1495-502 (2004).

Sialic acid decoys can also be selected solely on the basis of neuraminidase inhibition. For example, 4-difluoromethyl-2-methoxy-phenyl-α-ketoside of N-acetylneuraminic acid exhibits reversible inhibition neuraminidase influenza-infected MDCK cells (Kᵢ 8×10⁻⁵M). Barrere et al., 142 Arch. Virol. 1365-80 (1997). Reversible neuraminidase competitive inhibitors of influenza A and B include zanamivir, oseltamivir carboxylate (GS4071), and RWJ-270201 (BCX-1812). The half-time rate of dissociation from the active site of oseltamivir carboxylate neuraminidase is 33 to 60 minutes in cell culture. In comparison, the half-times for dissociation of A-315675 (5-[(1R,2S)-1-(acetylamino)-2-methoxy-2-methylpentyl]-4-[(1Z)-1-propenyl]-(4S,5R)-D-proline), a pyrrolidine-based compound, from influenza virus neuraminidase is 10 to 12 hours in MDCK cell culture. Kati et al., 46 Antimicrobial Ag. Chemother. 1014-21 (2002).

Chick embryo fibroblasts infected with influenza A (avian plague virus) contain a precursor glycoprotein that yields, after cleavage, the glycoproteins of the hemagglutinin. High concentrations of D-glucosamine and 2-deoxy-D-glucose inhibited the formation of hemagglutinin, neuraminidase, and mature virions. Analysis revealed that, under these conditions, all viral glycoproteins were missing. Instead of the glycoproteins, a single carbohydrate-free polypeptide chain of the glycoprotein precursor of the hemagglutinin was accumulated in infected cells. Klenk et al., 49 Virol. 723-34 (1972).

In one embodiment, the sialic acid decoy is introduced to the embryonated eggs concurrent with introduction of the RNA effector molecule(s) and infective virus. This avoids multiple exposures of the egg to possible contamination, but provides for a lag in viral infection while the RNA effector(s) contact the cell and modulate cellular activity.

Because sialic acid decoys can inhibit viral shed as well as initial infection, decoys can be used to inhibit viral particle release into cell media, such that the viral progeny can be retained in the host cells if this outcome is desired. Gubareva et al., 355 Lancet 872 (2000). Thus, in some embodiments, after a desired level of infection is achieved, defective (mis-enveloped) virus or other immunogenic agent can be recovered from collected cells.

In alternative embodiments, the binding of virus to sialic acid is used as a delivery mechanism to contact RNAi agents (e.g., RNA effector molecules) with the host cell. In this approach, RNA effector molecules are combined with or conjugated to sialic acids or derivatives thereof. In one embodiment, viral inoculum is mixed with RNA-effector-coupled sialic acid derivatives, such that a portion (but not all), of the hemagglutinin residues on the virus are complexed with sialic acid-siRNA conjugate. Unoccupied hemagglutinin residues bind with host cell sialic acids, and endocytosis ensues, thereby promoting the release of both the virus and the RNA effector molecule(s) into the host cell cytoplasm.

In other embodiments, sialic acids are incorporated into liposomal formulations with the siRNA. Liposomes expressing sialic acid residues have been used extensively in the study of influenza and cell-surface interactions. Reichert et al., 117 J. Am. Chem. Soc. 829-30 (1995); Spevak et al., 115 J. Am. chem. Soc. 1146-47 (1993). In an aspect of this embodiment, the siRNA-sialic acid-liposome formulation is mixed with influenza prior to inoculation of the egg.

Host Cell Viability

In some embodiments, the production of a biological product in a host cell is enhanced by introducing into the cell an additional RNA effector molecule that affects cell growth, cell division, cell viability, apoptosis, nutrient handling, and/or other properties related to cell growth and/or division within the cell. The target gene can also encode a host cell protein that directly or indirectly affects one or more aspects of the production of the biological product. Examples of target genes that affect the production of polypeptides include genes encoding proteins involved in the secretion, folding or post-translational modification of polypeptides and/or virus particles (e.g., glycosylation, deamidation, disulfide bond formation, methionine oxidation, or pyroglutamation); genes encoding proteins that influence a property or phenotype of the host cell (e.g., growth, viability, cellular pH, cell cycle progression, apoptosis, carbon metabolism or transport, lactate formation, susceptibility to viral infection or RNAi uptake, activity or efficacy); and genes encoding proteins that impair the production of a biological product by the host cell (e.g., a protein that binds or co-purifies with the biological product) (also genes encode proteins that interfere with the release of virus particles from the cell).

In some embodiments of the invention, the target gene encodes a host cell protein that indirectly affects the production of a biological product such that inhibiting expression of the target gene enhances production of the biological product. For example, the target gene can encode an abundantly expressed host cell protein that does not influence directly production of the biological product, but indirectly decreases its production, for example by utilizing cellular resources that could otherwise enhance production of the biological product.

For optimal production of a biological product in cell-based bioprocesses described herein, it is desirable to maximize cell viability. Accordingly, in one embodiment, production of a biological product is enhanced by modulating expression of a cell protein that affects apoptosis or cell viability, such as Bax (BCL2-associated X protein), for example; Bak (BCL2-antagonist/killer 1) (Bak, GeneID: 419912), LDHA (lactate dehydrogenase A) (LdhA, GeneID: 396221, modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154553-3154578 (sense) and SEQ ID NOs:3154579-3154604 (antisense)), LDHB (GeneID: 373997), BIK; BAD, BID, BIM, HRK, BCLG, HR, NOXA, PUMA, BOK (BCL2-related ovarian killer, GeneID: 995445), BOO, BCLB, CASP2 (apoptosis-related cysteine peptidase 2) (Casp2, GeneID: 395857), CASP3 (GeneID: 395476), CASP6 (GeneID: 395477), CASP7 (GeneID: 423901), CASP8 (GeneID: 395284), CASP9 (GeneID: 426970), CASP10 (GeneID: 424081), BCL2 (B-cell CLL/lymphoma 2) (Bc12, GeneID: 396282), p53 (GeneID: 396200), APAF1, HSP70 (GeneID: 423504), TRAIL (TRAIL-LIKE TNF-related apoptosis inducing ligand-like) (Trail, GeneID: 395283), BCL2L1 (BCL2-like 1) (Bc12L1, GeneID: 373954), BCL2L13 (BCL2-like 13 [apoptosis facilitator]) (Bc12113, GeneID: 418163), BCL2L14 (GeneID: 419096), FASLG (Fas ligand [TNF superfamily, member 6]), GeneID: 429064), DPF2 (D4, zinc and double PHD fingers family 2) (Dpf2, GeneID: 429064), AIFM2 (apoptosis-inducing factor mitochondrion-associated 2) (Aifm2, GeneID: 423720), AIFM3 (GeneID: 416999), STK17A (serine/threonine kinase 17a [apoptosis-inducing]) (Stk17A, GeneID: 420775), APITD1 (apoptosis-inducing, TAF9-like domain 1) (Apitdl, GeneID: 771417), SIVA1 (apoptosis-inducing factor) (Sival, GeneID: 423493), FAS (TNF receptor superfamily member 6, Fas, GeneID: 395274), TGFβ2 (transforming growth factor β2, TgfB2, GeneID: 421352), TGFBR1 (transforming growth factor, (3 receptor I, TgfR1, GeneID: 374094), LOC378902 (death domain-containing tumor necrosis factor receptor superfamily member 23) (GeneID: 378902), or BCL2A1 (BCL2-related protein A1, GeneID: 395673).

For example, the Bak protein is known to down-regulate cell apoptosis pathways. Thus, a RNA effector molecule(s) that target chicken Bak can be used to suppress apoptosis and increase product yield, and comprises a sense strand and an antisense strand wherein one strand comprises at least 16 contiguous nucleotides (e.g., at least 17, at least 18, at least 19 nucleotides) of the nucleotides in SEQ ID NOs:3154393-3154413 (sense) and SEQ ID NOs:3154414-3154434 (antisense). See also Suyama et al., S1 Nucl. Acids. Res. 207-08 (2001). A particular embodiment thus provides for a RNA effector molecule that targets the Bak gene.

Similarly, Bax protein is known to down-regulate cell apoptosis pathways. Thus, a RNA effector molecule(s) that target chicken Bax can be used to suppress apoptosis and increase product yield, and comprises a sense strand and an antisense strand wherein one strand comprises at least 16 contiguous nucleotides (e.g., at least 17, at least 18, at least 19 nucleotides) selected from the nucleotides in SEQ ID NOs:3154393-3154413 (sense) and SEQ ID NOs:315414-3154434 (antisense).

In some embodiments, administration of RNA effector molecule/s targeting at least one gene involved in apoptosis (e.g., Bak, Bax, caspases etc.) is followed by a administration of glucose to the egg in order to increase cell density and switch cells to a lactate utilization mode. In some embodiments the concentration of glucose is increased at least 2-fold, at least 3-fold, at least 4 fold, or at least 5-fold.

Another embodiment provides for a plurality of different RNA effector molecules is contacted with the cells in the egg to permit modulation of Bax, Bak and LDH expression. In another embodiment, RNA effector molecules targeting Bax and Bak are co-administered to a egg during production of the biological product and can optionally contain at least one additional RNA effector molecule or agent.

Alternatively, one can administer one RNA effector molecule at a time to the egg. In this manner, one can easily tailor the average percent inhibition desired for each target gene by altering the frequency of administration of a particular RNA effector molecule. For example, >80% inhibition of lactate dehydrogenase (LDH) may not always be necessary to significantly improve production of a biological product and under some conditions can even be detrimental to cell viability. Thus, one can contact a cell with an RNA effector molecule targeting LDH at a lower frequency (e.g., less often) than the frequency of contacting with the other RNA effector molecules (e.g., Bax/Bak). Alternatively, the cell can be contacted with an RNA effector molecule targeting LDH at a lower dosage (e.g., lower multiples over the IC₅₀) than the dosage for other RNA effector molecules (e.g., Bax/Bak). For ease of use and to prevent potential contamination it may be preferred to administer a cocktail of different RNA effector molecules, thereby reducing the number of doses required and minimizing the chance of introducing a contaminant to the egg.

The production of a biological product in cell-based bioprocesses described herein can also be optimized by targeting genes that have been identified through screens. These include, for example, PUSL1 (pseudouridylate synthase-like 1), TPST1 (tyrosylprotein sulfotransferase 1, Tpst1, GeneID: 417546), and WDR33 (WD repeat domain 33, GeneID: 424753) (see Brass et al., 139 Cell 1243-54 (2009)), Nod2 (nucleotide-binding oligomerization domain containing 2) (Sabbah et al., 10 Nat. Immunol. 1973-80 (2009)); MCT4 (solute carrier family 16, member 4 [monocarboxylic acid transporter 4], GeneID: 395383), ACRC (acidic repeat containing), GeneID:422202), AMELY, ATCAY (cerebellar, Cayman type [caytaxin], GeneID: 420094), ANP32B (acidic [leucine-rich] nuclear phosphoprotein 32 family member, GeneID: 420087), DEFA3, DHRS10, DOCK4 (dedicator of cytokinesis 4, GeneID: 417779), FAM106A, FKBP1B (FK506 binding protein 1B, GeneID: 395254), IRF3, KBTBD8 (kelch repeat and BTB [POZ] domain containing 8, GeneID: 416085), KIAA0753 (GeneID: 417681), LPGAT1 (lysophosphatidyl-glycerol acyltransferase 1, GeneID: 421375), MSMB (microseminoprotein (3, GeneID: 423773), NFS1 (nitrogen fixation 1 homolog, GeneID: 419133), NPIP, NPM3 (nucleophosmin/nucleoplasmin 3, GeneID: 770430), SCGB2A1, SERPINB7, SLC16A4 (solute carrier family 16, member 4 [monocarboxylic acid transporter 5], GeneID: 419809), SPTBN4 (spectrin, p, non-erythrocytic 4, GeneID: 430775), or TMEM146 (Krishnan et al., 2008).

Other target genes that can be affected to optimize biologics production include genes associated with cell cycle and/or cell proliferation, such as CDKN1B (cyclin-dependent kinase inhibitor 1B, p27, kip1, GeneID: 374106), a targert for which a siRNA against p27kip1 induces proliferation (Kikuchi et al., 47 Invest. Opthalmol. 4803-09 (2006)); or FOX01, a target for which a siRNA induces aortic endothelial cell proliferation (Fosbrink et al., J. Biol. Chem. 19009-18 (2006).

An additional gene associated with improved intracellular protein expression is FN1. Expression of FN1 can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154435-3154463 (sense) and 3154464-3154492 (antisense).

Reactive oxygen species (ROS) are toxic to host cells and can mediate non-specific oxidation, degradation and/or cleavage and other structural modifications of the biological product that lead to increased heterogeneity, decreased biological activity, lower recoveries, and/or other impairments to of biologics produced by methods provided herein. Accordingly, production of a biological product is enhanced by modulating expression of a pro-oxidant enzyme, such as a protein selected from the group consisting of: NAD(p)H oxidase, peroxidase such as a glutathione peroxidase (e.g., glutathione peroxidase 1, glutathione peroxidase 4, glutathione peroxidase 8 (putative), glutathione peroxidase 3, myeloperoxidase, constitutive neuronal nitric oxide synthase (cnNOS), xanthine oxidase (XO) and myeloperoxidase (MPO), 15-lipoxygenase-1, NADPH cytochrome c reductase, NAPH cytochrome c reductase, NADH cytochrome b5 reductase, and cytochrome P4502E1.

Additionally, protein production can be enhanced by modulating expression of a protein that affects the cell cycle of host cells, such as a cyclin (e.g., cyclin M4, cyclin J, cyclin T2, cyclin-dependent kinase inhibitor 1A (P21), cyclin-dependent kinase inhibitor 1B, cyclin M3, cyclin-dependent kinase inhibitor 2B (p15, inhibits CDK4), cyclin E2, S100 calcium binding protein A6 (calcyclin), cyclin-dependent kinase 5, regulatory subunit 1 (p35), cyclin T1, inhibitor of CDK, cyclin A1 interacting protein, or a cyclin dependent kinase (CDK). In some embodiments, the target CDK is CDK2A, which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154663-3154696 (sense) and SEQ ID NOs:3154697-3154730 (antisense). For example, in various embodiments, the expression of one or more proteins that affect cell cycle progression can be transiently modulated during the growth and/or production phases of heterologous protein production in order to enhance expression and recovery of heterologous proteins.

In addition, production of excess ammonia in bioprocessing is a common problem. High ammonia concentrations result in reduced cell and product yields depending on cell line and process conditions. Liberation of ammonia is thought to occur through the breakdown of glutamine to glutamate by glutaminase, and/or through the conversion of glutamate to a-ketoglutarate by glutamate dehydrogenase. Therefore, in one embodiment, biologics production can be enhanced by modulating expression of a protein that affects ammonia production, such as glutaminase or glutamine synthetase.

It is known that production of lactic acid in cells inhibits cell growth and influences metabolic pathways involved in glycolysis and glutaminolysis (Lao & Toth, 13 Biotech. Prog., 688-91 (1997)). The accumulation of lactate in cells is caused mainly by the incomplete oxidation of glucose to CO₂ and H₂O, in which most of the glucose is oxidized to pyruvate and finally converted to lactate by lactate dehydrogenase (LDH). The accumulation of lactic acid in cells is detrimental to achieving high cell density and viability. Accordingly, in one embodiment, immunogenic protein production is enhanced by modulating expression of a protein that affects lactate formation, such as lactate dehydrogenase A (LDHA). Hence, a particular embodiment provides for a RNA effector molecule that targets the LDHA1 gene LDHA, which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154553-3154578 (sense) and SEQ ID NOs:3154579-3154604 (antisense).

In one embodiment, a cell culture is treated as described herein with RNA effector molecules that permit modulation of Bax, Bak and LDH expression. In another embodiment, the RNA effector molecules targeting Bax, Bak and LDH can be administered in combination with one or more additional RNA effector molecules and/or agents. Provided herein is a cocktail of RNA effector molecules targeting Bax, Bak and LDH expression, which can optionally be combined with additional RNA effector molecules or other bioactive agents as described herein.

In some embodiments, production of a biological product is enhanced by modulating expression of a protein that affects cellular pH, such as LDH or lysosomal V-type ATPase. In some embodiments, production of a biological product is enhanced by modulating expression of cofilin (for example a muscle cofilin 2, or non-muscle cofilin-1).

In some embodiments, production of a biological product is enhanced by modulating expression of a protein that affects carbon metabolism or transport, such as GLUT1, GLUT2, GLUT3, GLUT4, PTEN, or LDH. For example, when the target is PTEN, the egg cell can be contacted with a RNA effector molecule comprising a sense strand and an antisense strand wherein one strand comprises at least 16 contiguous nucleotides (e.g., at least 17, at least 18, at least 19 nucleotides) of a nucleotide sequence selected from the group consisting of SEQ ID NOs:3154493-3154522 (sense) and SEQ ID NOs:3154523-3154552 (antisense).

In some embodiments, production of a biological product is enhanced by modulating expression of a protein that affects uptake or efficacy of an RNA effector molecule in host cells, such as ApoE, Mannose/GalNAc-receptor, and Eri1. In various embodiments, the expression of one or more proteins that affects RNAi uptake or efficacy in cells is modulated according to a method provided herein concurrently with modulation of one or more additional target genes, such as a target gene described herein, in order to enhance the degree and/or extent of modulation of the one or more additional target genes.

In some embodiments, the production of a biological product is enhanced by inducing a stress response in the host cells which causes growth arrest and increased productivity. A stress response can be induced, e.g., by limiting nutrient availability, increasing solute concentrations, or low temperature or pH shift, and oxidative stress. Along with increased productivity, stress responses can also have adverse effects on protein folding and secretion. In some embodiments, such adverse effects are ameliorated by modulating the expression of a target gene encoding a stress response protein, such as a protein that affects protein folding and/or secretion described herein.

In some embodiments, production of a biological product is enhanced by modulating expression of a protein that affects cytoskeletal structure, e.g., altering the equilibrium between monomeric and filamentous actin. In one embodiment the target gene encodes cofilin and a RNA effector molecule inhibits expression of cofilin. In one embodiment, at least one RNA effector molecule increases expression of a target gene selected from the group consisting of: cytoplasmic actin capping protein (CapZ), Ezrin (VIL2), and Laminin A.

The modulation of expression (e.g., inhibition) of a target gene by a RNA effector molecule can be further alleviated by introducing a second RNA effector molecule, wherein at least a portion of the second RNA effector molecule is complementary to a target gene encoding a protein that mediates RNAi in the host cell. For example, the modulation of expression of a target gene can be alleviated by introducing into the cell a RNA effector molecule that inhibits expression of an Argonaute protein (e.g., Argonaute-2) or other component of the RNAi pathway of the cell. In one embodiment, the biological product is transiently inhibited by contacting the cell with a first RNA effector molecule targeted to the biological product. The inhibition of expression of the biological product is then alleviated by introducing into the cell a second RNA effector molecule targeted against a gene encoding a protein of the RNAi pathway.

Additionally, the production of a desired biological product can be enhanced by introducing into the cell a RNA effector molecule during the production phase to modulate expression of a target gene encoding a protein that affects protein expression, post-translational modification, folding, secretion, and/or other processes related to production and/or recovery of the desired biological product. Alternatively, the production of a biological product is enhanced by introducing into the cell a RNA effector molecule which inhibits cell growth and/or cell division during the production phase.

Additional target genes include miRNA antagonists that can be used to determine if this is the basis of some viruses not growing well in cells, for example Dicer (dicer 1, ribonuclease type III), because knock-down of Dicer leads to a increase in the rate of infection (Matskevich et al., 88 J. Gen. Virol. 2627-35 (2007), the expression of which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3156059-3156106 (sense) and SEQ ID NOs:3156107-3156154 (antisense)); or ISRE (interferon-stimulated response element), as a decoy to titrate TFs away from ISRE-containing promoters.

A plurality of different RNA effector molecules are introduced into the egg and permit modulation of one or more target genes. In one embodiment, the RNA effector molecules are administered during production of the viral product. In another embodiment, a plurality of different RNA effector molecules is contacted with the cells in the egg to permit modulation of PTEN, CDKN2A, BAK1, FN1, LDHA, IFN, and/or IFNAR1 gene expression. The effector molecules can be co-administered during the virus production and can optionally contains an additional gene or agent.

When a plurality of different RNA effector molecules are used to modulate expression of one or more target genes the plurality of RNA effector molecules are contacted with the egg cells simultaneously or separately. In addition, each RNA effector molecule can have its own dosage regime. For example, one can prepare a composition comprising a plurality of RNA effector molecules are contacted with a cell. Alternatively, one can administer one RNA effector molecule at a time to the egg. In this manner, one can easily tailor the average percent inhibition desired for each target gene by altering the frequency of administration of a particular RNA effector molecule. For example, full inhibition (i.e., >80%) of lactate dehydrogenase (LDH) is not always necessary to significantly improve production of a viral product and under some conditions can even be detrimental to egg cell viability. Thus, one may desire to contact a cell with an RNA effector molecule targeting LDH at a lower frequency (e.g., less often) or at a lower dosage (e.g., lower multiples over the IC₅₀) than the dosage for other RNA effector molecules. For ease of use and to prevent potential contamination it may be preferred to administer a cocktail of different RNA effector molecules, thereby reducing the number of doses required and minimizing the chance of introducing a contaminant to the egg.

Additionally, protein production can be enhanced by modulating expression of a protein that affects the cell cycle of host cells, such as a cyclin (e.g., CDC2) or a cyclin dependent kinase (CDK). For example, the cyclin dependent kinase can be CDK2, CDK4, P10, P21, P27, p53, P57, p161NK4a, P14ARF, and CDK4. Thus, for example, the expression of one or more proteins that affect cell cycle progression can be transiently modulated during the growth and/or production phases of viral protein production in order to enhance expression and recovery of viral products. A particular embodiment provides for a RNA effector molecule that targets the CDKN1 gene.

Post-Translational Processing

Post-translational modifications can require additional bioprocess steps to separate modified and unmodified polypeptides, increasing costs and reducing efficiency of biologics production. Accordingly, in some embodiments, in production of a polypeptide agent in a cell is enhanced by modulating the expression of a target gene encoding a protein that affects post-translational modification. In additional embodiments, biologics production is enhanced by modulating the expression of a first target gene encoding a protein that affects a first post-translational modification, and modulating the expression of a second target gene encoding a protein that affects a second post-translational modification.

More specifically, proteins expressed in eukaryotic cells can undergo several post-translational modifications that can impair production and/or the structure, biological activity, stability, homogeneity, and/or other properties of the biological product. Many of these modifications occur spontaneously during cell growth and polypeptide expression and can occur at several sites, including the peptide backbone, the amino acid side-chains, and the amino and/or carboxyl termini of a given polypeptide. In addition, a given polypeptide can comprise several different types of modifications. For example, proteins expressed in avian and mammalian cells can be subject to acetylation, acylation, ADP-ribosylation, amidation, ubiquitination, methionine oxidation, disulfide bond formation, methylation, demethylation, sulfation, formation of cysteine, formation of pyroglutamate, formylation, gamma-carboxylation, hydroxylation, iodination, myristoylation, oxidation, proteolytic processing, phosphorylation, prenylation, racemization, glycosylation, gluconoylation, sequence mutations, N-terminal glutamine cyclization and deamidation, and asparagine deamidation. N-terminal asparagine deamidation can be reduced by contacting the cell with an RNA effector molecule targeting the N-terminal Asn amidase.

In some embodiments, protein production is enhanced by modulating expression of a target gene which encodes a protein involved in protein deamidation. Proteins can be deamidated via several pathways, including the cyclization and deamidation of N-terminal glutamine and deamidation of asparagine. Thus, in one embodiment, the protein involved in protein deamidation is N-terminal asparagine amidohydrolase. Protein deamidation can lead to altered structural properties, reduced potency, reduced biological activity, reduced efficacy, increased immunogenicity, and/or other undesirable properties and can be measured by several methods, including but not limited to, separations of proteins based on charge by, e.g., ion exchange chromatography, HPLC, isoelectric focusing, capillary electrophoresis, native gel electrophoresis, reversed-phase chromatography, hydrophobic interaction chromatography, affinity chromatography, mass spectrometry, or the use of L-isoaspartyl methyltransferase.

When the biological product comprises a glycoprotein, such as a viral product having viral surface membrane proteins or monoclonal antibody having glycosylated amino acid residues, biologics production can be enhanced by modulating expression of a target gene that encodes a protein involved in protein glycosylation. Glycosylation patterns are often important determinants of the structure and function of mammalian glycoproteins, and can influence the solubility, thermal stability, protease resistance, antigenicity, immunogenicity, serum half-life, stability, and biological activity of glycoproteins.

In various embodiments, the protein that affects glycosylation is selected from the group consisting of: dolichyl-diphosphooligosaccharide-protein glycosyltransferase, UDP glycosyltransferase, UDP-Gal:βGlcNAc β1,4-galactosyltransferase, UDP-galactose-ceramide galactosyltransferase, fucosyltransferase, protein O-fucosyltransferase, N-acetylgalactosaminytransferase, particularly T4, O-GlcNAc transferase, oligosaccharyl transferase, O-linked N-acetylglucosamine transferase, α-galactosidase, and β-galactosidase.

In further embodiments, production of a glycoprotein is enhanced by modulating expression of a sialidase or a sialytransferase enzyme. Terminal sialic acid residues of glycoproteins are particularly important determinants of glycoprotein solubility, thermal stability, resistance to protease attack, antigenicity, and specific activity. For example, when terminal sialic acid is removed from serum glycoproteins, the desialylated proteins have significantly decreased biological activity and lower circulatory half-lives relative to sialylated counterparts. The amount of sialic acid in a glycoprotein is the result of two opposing processes, i.e., the intracellular addition of sialic acid by sialytransferases and the removal of sialic acid by sialidases. Thus, in some embodiments, production of a glycoprotein is enhanced by inhibiting expression of a sialidase and/or activating expression of a sialytransferase.

In some embodiments, protein production is enhanced by modulating expression of a glutaminyl cyclase which catalyzes the intramolecular cyclization of N-terminal glutamine residues into pyroglutamic acid, liberating ammonia (pyroglutamation). Glutaminyl cyclase modulation can be accomplished by contacting the cell with an RNA effector molecule targeting the glutaminyl cyclase gene.

In some embodiments, production of proteins containing disulfide bonds is enhanced by modulating expression of a protein that affects disulfide bond oxidation, reduction, and/or isomerization, such as protein disulfide isomerase or sulfhydryl oxidase. Disulfide bond formation can be particularly problematic for the production of multi-subunit proteins or peptides in recombinant cells. Examples of multi-subunit proteins or peptides include receptors, extracellular matrix proteins, immunomodulators, such as MHC proteins, full chain antibodies and antibody fragments, enzymes, and membrane proteins.

In some embodiments, protein production is enhanced by modulating expression of a protein that affects methionine oxidation. Reactive oxygen species (ROS) can oxidize methionine (Met) to methionine sulfoxide (MetO), resulting in increased degradation and product heterogeneity, and reduced biological activity and stability. In some embodiments, the target gene encodes a methionine sulfoxide reductase, which catalyzes the reduction of MetO residues back to methionine.

Biological products (including some live attenuated viruses) produced in eggs on an industrial-scale are typically secreted by egg cells and recovered and purified from the surrounding extracellular milieu. In general, the rate of protein production and the yield of recovered protein is directly related to the rate of protein folding and secretion by the host cells. For example, an accumulation of misfolded proteins in the endoplasmic reticulum (ER) of host cells can slow or stop secretion via the unfolded protein response (UPR) pathway. The UPR is triggered by stress-sensing proteins in the ER membrane which detect excess unfolded proteins. UPR activation leads to the upregulation of chaperone proteins (e.g., Bip) which bind to misfolded proteins and facilitate proper folding. UPR activation also upregulates the transcription factors XBP-1 and CHOP. CHOP generally functions as a negative regulator of cell growth, differentiation and survival, and its upregulation via the UPR causes cell cycle arrest and increases the rate of protein folding and secretion to clear excess unfolded proteins from the cell. Hence, cell cycle can be promoted initially, then repressed during virus production phase to increase viral product yield. An increase the rate of immunogenic protein secretion by the host cells can be measured by, e.g., monitoring the amount of protein present in the egg over time.

The present invention provides methods for enhancing the production of a secreted polypeptide in egg cells by modulating expression of a target gene which encodes a protein that affects protein secretion by the cells in the embryonated egg. In some embodiments, the target gene encodes a protein of the UPR pathway, such as IRE1, PERK, ATF4, ATF6, eIF2a, GRP78, GRP94, calreticulin, or a variant thereof, or a protein that regulates the UPR pathway, such as a transcriptional control element (e.g., the cis-acting UPR element (UPRE)). In some embodiments, the protein that affects protein secretion is selected from the group consisting of: gamma-secretase, p115, a signal recognition particle (SRP) protein, secretin, and a kinase (e.g., MEK).

In some embodiments, the protein that affects protein secretion is a molecular chaperone selected from the group consisting of: Hsp40, HSP47, HSP60, Hsp70, HSP90, HSP100, protein disulfide isomerase, peptidyl prolyl isomerase, calnexin, Erp57, and BAG-1.

The production of biological products in eggs can be negatively affected by proteins which have an affinity for the biological product or a molecule or factor that binds specifically to the biological product. For example, a number of heterologous proteins have been shown to bind the glycoproteins heparin and heparan sulfate at host cell surfaces. This can lead to the co-purification of heparin, heparan sulfate, and/or heparin/heparan sulfate-binding proteins with recombinant protein products, decreasing yield and reducing homogeneity, stability, biological activity, and/or other properties of the recovered proteins. In one embodiment, the level of heparin and/or heparan sulfate is reduced by modulating expression of a host cell enzyme involved in the production of heparin and/or heparan sulfate, such as a host cell xylotransferase.

In some embodiments, for example when a biological product is viral, such as an influenza virus, target genes are those involved in reducing sialic acid from the host cell surface, which reduces virus binding, and therefore increases recovery of the virus from the extracellular milieu (i.e., less virus remains stuck on host cell membranes). These targets include: solute carrier family 35 (CMP-sialic acid transporter) member A1 (SLC35A1, which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154345-3154368 (sense) and SEQ ID NOs:3154369-3154392 (antisense)); solute carrier family 35 (UDP-galactose transporter), member A2 (SLC35A2); UDP-N-acetylglucosamine 2-epimerase/N-acetylmannosamine kinase (GNE), which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154297-3154320 (sense) and SEQ ID NOs:3154321-3154344 (antisense); cytidine monophospho-N-acetylneuraminic acid synthetase (Cmas), which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154249-3154272 (sense) and SEQ ID NOs:3154273-3154296 (antisense)); UDP-Gal:βGlcNAc β1,4-galactosyltransferase (B4GalT1), which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154153-3154176 (sense) and SEQ ID NOs:3154177-3154200 (antisense); and UDP-Gal:βGlcNAc β1,4-galactosyltransferase, polypeptide 6 (B4GalT6), which can be modulated by use of a corresponding RNA effector molecule comprising a sense strand and an antisense strand wherein one strand comprises at least 16 contiguous nucleotides (e.g., at least 17, at least 18, at least 19 nucleotides) of the nucleotides in SEQ ID NOs:3154201-3154224 (sense) and SEQ ID NOs:3154225-3154248 (antisense).

Additional targets can include those involved in host sialidase in avian cells (see Wang et al., 10 BMC Genomics 512 (2009)), because influenzae binds to cell surface sialic acid residues, thus decreased sialidase can increase the rate of infection or purification: NEU2 sialidase 2 (cytosolic sialidase) (GeneID: 430542) and NEU3 sialidase 3 (membrane sialidase) (GeneID: 68823). Additional target genes include miRNA antagonists that can be used to determine if this is the basis of some viruses not growing well in cells, for example Dicer (dicer 1, ribonuclease type III) because knock-down of Dicer leads to a modest increase in the rate of infection (Matskevich et al., 88 J. Gen. Virol. 2627-35 (2007)); or ISRE (interferon-stimulated response element), as a decoy titrate TFs away from ISRE-containing promoters.

The use of bioprocesses for the manufacture of biological products such as polypeptides at an industrial scale is often confounded by the presence of pathogens, such as active viral particles, and other adventitious agents (e.g., prions), often necessitating the use of expensive and time consuming steps for their detection, removal (e.g., viral filtration) and/or inactivation (e.g., heat treatment) to conform to regulatory procedures. Such problems can be exacerbated due to the difficulty in detecting and monitoring the presence of such viruses. Accordingly, in some embodiments, methods are provided for enhancing production of a biological product by modulating expression of a target gene affecting the susceptibility of a host cell to pathogenic infection. For example, in some embodiments, the target gene is a host cell protein that mediates viral infectivity, such as the transmembrane proteins XPR1, RDR, Flyer, CCRS, CXCR4, CD4, Pit1, and Pit2.

Although a target sequence is generally 10 to 30 nucleotides in length, there is wide variation in the suitability of particular sequences in this range for directing cleavage of any given target RNA. Various software packages and the guidelines set out herein provide guidance for the identification of optimal target sequences for any given gene target, but an empirical approach can also be taken in which a “window” or “mask” of a given size (as a non-limiting example, 21 nucleotides) is literally or figuratively (including, e.g., in silico) placed on the target RNA sequence to identify sequences in the size range that can serve as target sequences. By moving the sequence “window” progressively one nucleotide upstream or downstream of an initial target sequence location, the next potential target sequence can be identified, until the complete set of possible sequences is identified for any given target size selected. This process, coupled with systematic synthesis and testing of the identified sequences (using assays as described herein or as known in the art) to identify those sequences that perform optimally can identify those RNA sequences that, when targeted with a RNA effector molecule agent, mediate the best inhibition of target gene expression. Thus, although the sequences identified herein represent effective target sequences, it is contemplated that further optimization of inhibition efficiency can be achieved by progressively “walking the window” one nucleotide upstream or downstream of the given sequences to identify sequences with equal or better inhibition characteristics.

Further, it is contemplated that for any oligonucleotide identified herein further optimization could be achieved by systematically either adding or removing nucleotides to generate longer or shorter sequences and testing those and sequences generated by walking a window of the longer or shorter size up or down the target RNA from that point. Coupling this approach to generating new candidate targets with testing for effectiveness of RNA effector molecules based on those target sequences in an inhibition assay as known in the art or as described herein can lead to further improvements in the efficiency of inhibition. Further still, such optimized sequences can be adjusted by, e.g., the introduction of modified nucleotides as described herein or as known in the art, addition or changes in overhang, or other modifications as known in the art and/or discussed herein to further optimize the molecule (e.g., increasing serum stability or circulating half-life, increasing thermal stability, enhancing transmembrane delivery, targeting to a particular location or cell type, increasing interaction with silencing pathway enzymes, increasing release from endosomes, etc.) as an expression inhibitor.

### III. BIOCONTAMINATION

Evidence of avian leukosis virus and endogenous avian virus have been identified in chicken cell-derived vaccines. Tsang et al., 1999. Hence, an embodiment of the present invention provide for the use of RNA effector molecules to inhibit the expression of endogenous avian viruses. Such endogenous virus include endogenous retrovirus (ERV) avian Class III, Spuma-like ERVs gg01-chr7-7163462, gg01-chrU-52190725 and gg01-Chr4-48130894; avian ERVs ALV (ALV pol GeneID: 1491910, ALV p2, GeneID: 1491909, ALV p10, GeneID: 1491908, and ALV env, GeneID: 1491907; ALV transmembrane protein, an, GeneID: 1491906; ALV trans-acting factor, GeneID: 1491911) and gg01-chr1-15168845; avian Intermediate β-like ERVs gg01-chr4-77338201, gg01-ChrU-163504869, and gg01-chr7-5733782.

Latent DNA viruses that can be targeted by the methods of the present invention include adenoviruses. For example, avian adenovirus and adenovirus-associated virus (AAV) proteins have been produced by specific-pathogen-free chicks, indicating that avian AAV can exist as a latent infection in the germ line of chickens. Sadasiv et al., 33 Avian Dis. 125-33 (1989); see also Katano et al., 36 Biotechniq. 676-80 (2004). In some embodiments of the invention, the target gene is a latent DNA virus.

“Adventitious virus” or “adventitious viral agent” refers to a virus contaminant present within a biological product, including, for example, vaccines, cell lines and other cell-derived products. Regarding vaccine products, for example, exogenous, adventitious ALV was found in commercial Marek's Disease vaccines propagated in chicken and duck embryo fibroblast cultures by different manufacturers. Moreover, some of these vaccines were also contaminated with endogenous avian leukosis virus (ALV). Fadly et al., 50 Avian Diseases 380-85 (2006); Zavala & Cheng, 50 Avian Diseases 209-15 (2006).

### IV. RNA EFFECTOR MODIFICATION

In some embodiments of the present invention, an oligonucleotide (e.g., a RNA effector molecule) is chemically modified to enhance stability or other beneficial characteristics.

In one embodiment the RNA effector molecule is not chemically modified. Oligonucleotides can be modified to prevent rapid degradation of the oligonucleotides by endo- and exo-nucleases and avoid undesirable off-target effects.

The nucleic acids featured in the invention can be synthesized and/or modified by methods well established in the art, such as those described in CURRENT PROTOCOLS IN NUCL. ACID CHEM. (Beaucage et al., eds., John Wiley & Sons, Inc., NY). Modifications include, for example, (a) end modifications, e.g., 5′ end modifications (phosphorylation, conjugation, inverted linkages, etc.), or 3′ end modifications (conjugation, DNA nucleotides, inverted linkages, etc.); (b) base modifications, e.g., replacement with stabilizing bases, destabilizing bases, or bases that base pair with an expanded repertoire of partners, removal of bases (abasic nucleotides), or conjugated bases; (c) sugar modifications (e.g., at the 2′ position or 4′ position) or replacement of the sugar; as well as (d) internucleoside linkage modifications, including modification or replacement of the phosphodiester linkages. Specific examples of oligonucleotide compounds useful in this invention include, but are not limited to RNAs containing modified backbones or no natural internucleoside linkages. RNAs having modified backbones include, among others, those that do not have a phosphorus atom in the backbone. Specific examples of oligonucleotide compounds useful in this invention include, but are not limited to oligonucleotides containing modified or non-natural internucleoside linkages. Oligonucleotides having modified internucloside linkages include, among others, those that do not have a phosphorus atom in the internucleoside linkage. For the purposes of this specification, and as sometimes referenced in the art, modified oligonucleotides that do not have a phosphorus atom in their internucleoside linkage(s) can also be considered to be oligonucleosides. In particular embodiments, the modified oligonucleotides will have a phosphorus atom in its internucleoside linkage(s). For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified internucleoside linkages include (e.g., RNA backbones) include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Representative patents that teach the preparation of the above phosphorus-containing linkages include, but are not limited to, U.S. Pat. No. 3,687,808; No. 4,469,863; No. 4,476,301; No. 5,023,243; No. 5,177,195; No. 5,188,897; No. 5,264,423; No. 5,276,019; No. 5,278,302; No. 5,286,717; No. 5,321,131; No. 5,399,676; No. 5,405,939; No. 5,453,496; No. 5,455,233; No. 5,466,677; No. 5,476,925; No. 5,519,126; No. 5,536,821; No. 5,541,316; No. 5,550,111; No. 5,563,253; No. 5,571,799; No. 5,587,361; No. 5,625,050; No. 6,028,188; No. 6,124,445; No. 6,160,109; No. 6,169,170; No. 6,172,209; No. 6, 239,265; No. 6,277,603; No. 6,326,199; No. 6,346,614; No. 6,444,423; No. 6,531,590; No. 6,534,639; No. 6,608,035; No. 6,683,167; No. 6,858,715; No. 6,867,294; No. 6,878,805; No. 7,015,315; No. 7,041,816; No. 7,273,933; No. 7,321,029; and No. RE39464.

Modified oligonucleotide internucleoside linakges (e.g., RNA backbones) that do not include a phosphorus atom therein have internucleoside linkages that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative patents that teach the preparation of the above oligonucleosides include, but are not limited to, U.S. Pat. No. 5,034,506; No. 5,166,315; No. 5,185,444; No. 5,214,134; No. 5,216,141; No. 5,235,033; No. 5,64,562; No. 5,264,564; No. 5,405,938; No. 5,434,257; No. 5,466,677; No. 5,470,967; No. 5,489,677; No. 5,541,307; No. 5,561,225; No. 5,596,086; No. 5,602,240; No. 5,608,046; No. 5,610,289; No. 5,618,704; No. 5,623,070; No. 5,663,312; No. 5,633,360; No. 5,677,437; and No. 5,677,439.

Oligonucleotides can be modified to prevent rapid degradation of the oligonucleotides by endo- and exo-nucleases and avoid undesirable off-target effects. See, e.g., U.S. Patent Application Pub. No. 2009/0062225. Different chemical strategies can be employed for exo/endo light” modifications, including (a) exo/endo light sense strand: 2′-O-methyl at all pyrimidines, PTO between nucleotides 20 and 21 (counting from 5′-end), dTdT at 3′-end (nucleotides 20 and 21), exo/endo light antisense strand: 2′-O-methyl at pyrimidines in 5′-UA-3′ and 5′-CA-3′ motifs, PTO between nucleotides 20 and 21 (counting from 5′-end), dTdT at 3′-end (nucleotides 20 and 21); exo/endo light plus 2′-O-methyl in position 2 of antisense strand (only if no 5′-UA-3′ and 5′-CA-3′ at 5′-end, otherwise already covered by exo/endo light); (c) exo/endo light plus 2′-O-methyl in position 2 of sense strand (only if no pyrimidine in position 2, otherwise already covered by exo/endo light); and (d) exo/endo light plus 2′-O-methyl in position 2 of sense and antisense strand (only if not already covered by (a), (b), and (c)).

In other modified oligonucleotides suitable or contemplated for use in RNA effector molecules, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative patents that teach the preparation of PNA compounds include, but are not limited to, U.S. Pat. No. 5,539,082; No. 5,714,331; and No. 5,719,262. Further teaching of PNA compounds can be found, for example, in Nielsen et al., 254 Science 1497-1500 (1991).

Some embodiments featured in the invention include oligonucleotides with phosphorothioate internucleoside linkages and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester internucleoside linkage is represented as —O—P—O—CH₂—] (see U.S. Pat. No. 5,489,677), and amide backbones (see U.S. Pat. No. 5,602,240). In some embodiments, the oligonucleotides featured herein have morpholino backbone structures (see U.S. Pat. No. 5,034,506).

Modified oligonucleotides can also contain one or more substituted sugar moieties. The RNA effector molecules, e.g., dsRNAs, featured herein can include one of the following at the 2′ position: H (deoxyribose); OH (ribose); F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to 10, inclusive. In some embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide (e.g., a RNA effector molecule), or a group for improving the pharmacodynamic properties of an oligonucleotide (e.g., a RNA effector molecule), and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., 78 Helv. Chim. Acta 486-504 (1995)), i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the oligonucleotide, particularly the 3′ position of the sugar on the 3′ terminal nucleotide or in 2′-5′ linked oligonucleotide and the 5′ position of 5′ terminal nucleotide. Oligonucletodides can also have sugar mimetics such as cyclobutyl moieties in place of the pentofuranosyl sugar. Representative patents that teach the preparation of such modified sugar structures include, but are not limited to, U.S. Pat. No. 4,981,957; No. 5,118,800; No. 5,319,080; No. 5,359,044; No. 5,393,878; No. 5,446,137; No. 5,466,786; No. 5,514,785; No. 5,519,134; No. 5,567,811; No. 5,576,427; No. 5,591,722; No. 5,597,909; No. 5,610,300; No. 5,627,053; No. 5,639,873; No. 5,646,265; No. 5,658,873; No. 5,670,633; and No. 5,700,920, certain of which are commonly owned with the instant application.

An oligonucleotide (e.g., a RNA effector molecule) can also include nucleobase (often referred to in the art simply as “base”) modifications or substitutions. As used herein, “unmodified” or “natural” nucleobases include the purine bases adenine (A) and guanine (G), and the pyrimidine bases cytosine (C) and uracil (U).

Modified nucleobases include other synthetic and natural nucleobases such as as inosine, xanthine, hypoxanthine, nubularine, isoguanisine, tubercidine, 2-(halo)adenine, 2-(alkyl)adenine, 2-(propyl)adenine, 2 (amino)adenine, 2-(aminoalkyl)adenine, 2 (aminopropyl)adenine, 2 (methylthio) N6 (isopentenyl)adenine, 6 (alkyl)adenine, 6 (methyl)adenine, 7 (deaza)adenine, 8 (alkenyl)adenine, 8-(alkyl)adenine, 8 (alkynyl)adenine, 8 (amino)adenine, 8-(halo)adenine, 8-(hydroxyl)adenine, 8 (thioalkyl)adenine, 8-(thiol)adenine, N6-(isopentyl)adenine, N6 (methyl)adenine, N6, N6 (dimethyl)adenine, 2-(alkyl)guanine,2 (propyl)guanine, 6-(alkyl)guanine, 6 (methyl)guanine, 7 (alkyl)guanine, 7 (methyl)guanine, 7 (deaza)guanine, 8 (alkyl)guanine, 8-(alkenyl)guanine, 8 (alkynyl)guanine, 8-(amino)guanine, 8 (halo)guanine, 8-(hydroxyl)guanine, 8 (thioalkyl)guanine, 8-(thiol)guanine, N (methyl)guanine, 2-(thio)cytosine, 3 (deaza) 5 (aza)cytosine, 3-(alkyl)cytosine, 3 (methyl)cytosine, 5-(alkyl)cytosine, 5-(alkynyl)cytosine, 5 (halo)cytosine, 5 (methyl)cytosine, 5 (propynyl)cytosine, (propynyl)cytosine, 5 (trifluoromethyl)cytosine, 6-(azo)cytosine, N4 (acetyl)cytosine, 3 (3 amino-3 carboxypropyl)uracil, 2-(thio)uracil, 5 (methyl) 2 (thio)uracil, 5 (methylaminomethyl)-2 (thio)uracil, 4-(thio)uracil, 5 (methyl) 4 (thio)uracil, 5 (methylaminomethyl)-4 (thio)uracil, 5 (methyl) 2,4 (dithio)uracil, 5 (methylaminomethyl)-2,4 (dithio)uracil, 5 (2-aminopropyl)uracil, 5-(alkyl)uracil, 5-(alkynyl)uracil, 5-(allylamino)uracil, 5 (aminoallyl)uracil, (aminoalkyl)uracil, 5 (guanidiniumalkyl)uracil, 5 (1,3-diazole-1-alkyl)uracil, 5-(cyanoalkyl)uracil, 5-(dialkylaminoalkyl)uracil, 5 (dimethylaminoalkyl)uracil, 5-(halo)uracil, 5-(methoxy)uracil, uracil-5 oxyacetic acid, 5 (methoxycarbonylmethyl)-2-(thio)uracil, (methoxycarbonyl-methyl)uracil, 5 (propynyl)uracil, 5 (propynyl)uracil, 5 (trifluoromethyl)uracil, 6 (azo)uracil, dihydrouracil, N3 (methyl)uracil, 5-uracil (i.e., pseudouracil), 2 (thio)pseudouracil, 4 (thio)pseudouracil,2,4-(dithio)psuedouracil,5-(alkyl)pseudouracil, 5-(methyl)pseudouracil, 5-(alkyl)-2-(thio)pseudouracil, 5-(methyl)-2-(thio)pseudouracil, 5-(alkyl)-4 (thio)pseudouracil, 5-(methyl)-4 (thio)pseudouracil, 5-(alkyl)-2,4 (dithio)pseudouracil, 5-(methyl)-2,4 (dithio)pseudouracil, 1 substituted pseudouracil, 1 substituted 2(thio)-pseudouracil, 1 substituted 4 (thio)pseudouracil, 1 substituted 2,4-(dithio)pseudouracil, 1 (aminocarbonylethylenyl)-pseudouracil, 1 (aminocarbonylethylenyl)-2(thio)-pseudouracil, 1 (aminocarbonylethylenyl)-4 (thio)pseudouracil, 1 (aminocarbonylethylenyl)-2,4-(dithio)pseudouracil, 1 (aminoalkylaminocarbonylethylenyl)-pseudouracil, 1 (aminoalkylamino-carbonylethylenyl)-2(thio)-pseudouracil, 1 (aminoalkylaminocarbonylethylenyl)-4 (thio)pseudouracil, 1 (aminoalkylaminocarbonylethylenyl)-2,4-(dithio)pseudouracil, 1,3-(diaza)-2-(oxo)-phenoxazin-1-yl, 1-(aza)-2-(thio)-3-(aza)-phenoxazin-1-yl, 1,3-(diaza)-2-(oxo)-phenthiazin-1-yl, 1-(aza)-2-(thio)-3-(aza)-phenthiazin-1-yl, 7-substituted 1,3-(diaza)-2-(oxo)-phenoxazin-1-yl, 7-substituted 1-(aza)-2-(thio)-3-(aza)-phenoxazin-1-yl, 7-substituted 1,3-(diaza)-2-(oxo)-phenthiazin-1-yl, 7-substituted 1-(aza)-2-(thio)-3-(aza)-phenthiazin-1-yl, 7-(aminoalkylhydroxy)-1,3-(diaza)-2-(oxo)-phenoxazin-1-yl, 7-(aminoalkylhydroxy)-1-(aza)-2-(thio)-3-(aza)-phenoxazin-1-yl, 7-(aminoalkylhydroxy)-1,3-(diaza)-2-(oxo)-phenthiazin-1-yl, 7-(aminoalkylhydroxy)-1-(aza)-2-(thio)-3-(aza)-phenthiazin-1-yl, 7-(guanidiniumalkylhydroxy)-1,3-(diaza)-2-(oxo)-phenoxazin-1-yl, 7-(guanidiniumalkylhydroxy)-1-(aza)-2-(thio)-3-(aza)-phenoxazin-1-yl, 7-(guanidiniumalkyl-hydroxy)-1,3-(diaza)-2-(oxo)-phenthiazin-1-yl, 7-(guanidiniumalkylhydroxy)-1-(aza)-2-(thio)-3-(aza)-phenthiazin-1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenzyl, tetracenyl, pentacenyl, difluorotolyl, 4-(fluoro)-6-(methyl)benzimidazole, 4-(methyl)benzimidazole, 6-(azo)thymine, 2-pyridinone, 5 nitroindole, 3 nitropyrrole, 6-(aza)pyrimidine, 2 (amino)purine, 2,6-(diamino)purine, 5 substituted pyrimidines, N2-substituted purines, N6-substituted purines, 06-substituted purines, substituted 1,2,4-triazoles, pyrrolo-pyrimidin-2-on-3-yl, 6-phenyl-pyrrolo-pyrimidin-2-on-3-yl, para-substituted-6-phenyl-pyrrolo-pyrimidin-2-on-3-yl, ortho-substituted-6-phenyl-pyrrolo-pyrimidin-2-on-3-yl, bis-ortho-substituted-6-phenyl-pyrrolo-pyrimidin-2-on-3-yl, para-(aminoalkylhydroxy)-6-phenyl-pyrrolo-pyrimidin-2-on-3-yl, ortho-(aminoalkylhydroxy)-6-phenyl-pyrrolo-pyrimidin-2-on-3-yl, bis-ortho-(aminoalkylhydroxy)-6-phenyl-pyrrolo-pyrimidin-2-on-3-yl, pyridopyrimidin-3-yl, 2-oxo-7-amino-pyridopyrimidin-3-yl, 2-oxo-pyridopyrimidine-3-yl, or any O-alkylated or N-alkylated derivatives thereof. Modified nucleobases also include natural bases that comprise conjugated moieties, e.g., a ligand.

Further nucleobases include those disclosed in U.S. Pat. No. 3,687,808; MODIFIED NUCLEOSIDES BIOCHEM., BIOTECH. & MEDICINE (Herdewijn, ed., Wiley-VCH, 2008); WO 2009/120878; CONCISE ENCYCLOPEDIA OF POLYMER SCIENCE & ENGIN. 858-59 (Kroschwitz ed., John Wiley & Sons, 1990); Englisch et al., 30 Angewandte Chemie, Intl. Ed. 613 (1991); Sanghvi, 15 DSRNA RES. & APPLS. 289-302 (Crooke & Lebleu, eds., CRC Press, Boca Raton, Fla., 1993). Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, 276-78 (1993)), and are exemplary base substitutions, even more particularly when combined with 2′-β-methoxyethyl sugar modifications.

Representative patents that teach the preparation of certain of the above noted modified nucleobases as well as other modified nucleobases include, but are not limited to, the above noted U.S. Pat. No. 3,687,808; No. 4,845,205; No. 5,130,30; No. 5,134,066; No. 5,175,273; No. 5,367,066; No. 5,432,272; No. 5,457,191No. 5,457,187; No. 5,459,255; No. 5,484,908; No. 5,502,177; No. 5,525,711; No. 5,552,540; No. 5,587,469; No. 5,594,121, No. 5,596,091; No. 5,614,617; No. 5,681,941; No. 6,015,886; No. 6,147,200; No. 6,166,197; No. 6,222,025; No. 6,235,887; No. 6,380,368; No. 6,528,640; No. 6,639,062; No. 6,617,438; No. 7,045,610; No. 7,427,672; and No. 7,495,088; and No. 5,750,692.

The oligonucleotides can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to oligonucleotide molecules has been shown to increase oligonucleotide molecule stability in serum, and to reduce off-target effects. Elmen et al., 33 Nucl. Acids Res. 439-47 (2005); Mook et al., 6 Mol. Cancer. Ther. 833-43 (2007); Grunweller et al., 31 Nucl. Acids Res. 3185-93 (2003); U.S. Pat. No. 6,268,490; No. 6,670,461; No. 6,794,499; No. 6,998,484; No. 7,053,207; No. 7,084,125; No. 7,399,845.

In certain instances, the oligonucleotides of a RNA effector molecule can be modified by a non-ligand group. A number of non-ligand molecules have been conjugated to oligonucleotides in order to enhance the activity, cellular distribution or cellular uptake of the oligonucleotides, and procedures for performing such conjugations are available in the scientific literature. Such non-ligand moieties have included lipid moieties, such as cholesterol (Kubo et al., 365 Biochem. Biophys. Res. Comm. 54-61 (2007)); Letsinger et al., 86 PNAS 6553 (1989)); cholic acid (Manoharan et al., 1994); a thioether, e.g., hexyl-5-tritylthiol (Manoharan et al., 1992; Manoharan et al., 1993); a thiocholesterol (Oberhauser et al., 1992); an aliphatic chain, e.g., dodecandiol or undecyl residues (Saison-Behmoaras et al., 1991; Kabanov et al., 259 FEBS Lett. 327 (1990); Svinarchuk et al., 75 Biochimie 75 (1993)); a phospholipid, e.g., di-hexadecyl-rac-glycerol or triethylammonium 1,2-di-O-hexadecyl-rac-glycero-3-H-phosphonate (Manoharan et al., 1995); Shea et al., 18 Nucl. Acids Res. 3777 (1990)); a polyamine or a polyethylene glycol chain (Manoharan et al., Nucleosides & Nucleotides, 1995); or adamantane acetic acid (Manoharan et al., Tetrahedron Lett., 1995); a palmityl moiety (Mishra et al., 1995); or an octadecylamine or hexylamino-carbonyl-oxycholesterol moiety (Crooke et al., 1996). Representative United States patents that teach the preparation of such RNA conjugates have been listed herein. Typical conjugation protocols involve the synthesis of an oligonucleotide bearing an aminolinker at one or more positions of the sequence. The amino group is then reacted with the molecule being conjugated using appropriate coupling or activating reagents. The conjugation reaction can be performed either with the RNA still bound to the solid support or following cleavage of the RNA, in solution phase. Purification of the RNA conjugate by HPLC typically affords the pure conjugate.

Nucleic acid sequences of exemplary RNA effector molecules are represented below using standard nomenclature, and specifically the abbreviations of Table 2:

TABLE 2
Abbreviations of nucleotide monomers used
in nucleic acid sequence representation.

| Abbreviation | Nucleotide(s)* |
|---|---|
| A | adenosine |
| C | cytidine |
| G | guanosine |
| T | thymidine |
| U | uridine |
| N | any nucleotide (G, A, C, T or U) |
| a | 2′-O-methyladenosine |
| c | 2′-O-methylcytidine |
| g | 2′-O-methylguanosine |
| u | 2′-O-methyluridine |
| dT | 2′-deoxythymidine |
| s | phosphorothioate linkage |

*These monomers, when present in an oligonucleotide, are mutually linked by 5′-3′-phosphodiester bonds.

Ligands

Another modification of the oligonucleotides (e.g., of a RNA effector molecule) featured in the invention involves chemically linking to the oligonucleotide one or more ligands, moieties or conjugates that enhance the activity, cellular distribution or cellular uptake of the oligonucleotide. Such moieties include but are not limited to lipid moieties such as a cholesterol moiety (Letsinger et al., 86 PNAS 6553-56 (1989); cholic acid (Manoharan et al., 4 Biorg. Med. Chem. Let. 1053-60 (1994)); a thioether, e.g., beryl-5-tritylthiol (Manoharan et al., 660 Ann. NY Acad. Sci. 306309 (1992); Manoharan et al., 3 Biorg. Med. Chem. Let. 2765-70 (1993)); a thiocholesterol (Oberhauser et al., 20 Nucl. Acids Res. 533-38 (1992)); an aliphatic chain, e.g., dodecandiol or undecyl residues (Saison-Behmoaras et al., 10 EMBO J. 1111-18 (1991); Kabanov et al., 259 FEBS Lett. 327-30 (1990); Svinarchuk et al., 75 Biochimie 49-54 (1993)); a phospholipid, e.g., di-hexadecyl-rac-glycerol or triethyl-ammonium 1,2-di-O-hexadecyl-rac-glycero-3-phosphonate (Manoharan et al., 36 Tetrahedron Lett. 3651-54 (1995); Shea et al., 18 Nucl. Acids Res. 3777-83 (1990)); a polyamine or a polyethylene glycol chain (Manoharan et al., 14 Nucleosides & Nucleotides 969-73 (1995)); or adamantane acetic acid (Manoharan et al., Tetrahedron Lett., 1995); a palmityl moiety (Mishra et al., 1264 Biochim. Biophys. Acta 229-37 (1995)); or an octadecylamine or hexylamino-carbonyloxycholesterol moiety (Crooke et al., 227 J. Pharmacol. Exp. Ther. 923-37 (1996)).

In one embodiment, a ligand alters the distribution, targeting or lifetime of a RNA effector molecule agent into which it is incorporated. In some embodiments a ligand provides an enhanced affinity for a selected target, e.g., molecule, cell or cell type, compartment, e.g., a cellular or organ compartment, tissue, organ or region of the body, as, e.g., compared to a species absent such a ligand. Ideally, ligands will not take part in duplex pairing in a duplexed nucleic acid.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example polyamines include polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an -helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic.

Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases (e.g., EDTA), lipophilic molecules, e.g, cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid,O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the RNA effector molecule agent into the cell, for example, by disrupting the cell's cytoskeleton, e.g., by disrupting the cell's microtubules, microfilaments, and/or intermediate filaments. The drug can be, for example, taxol, vincristine, vinblastine, cytochalasin, nocodazole, japlakinolide, latrunculin A, phalloidin, swinholide A, indanocine, or myoservin.

An example ligand is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, Naproxen or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid based ligand can be used to modulate, e.g., control the binding of the conjugate to a target tissue. For example, a lipid or lipid-based ligand that binds to HSA more strongly will be less likely to be targeted to the kidney and therefore less likely to be cleared from the embryo. A lipid or lipid-based ligand that binds to HSA less strongly can be used to target the conjugate to the kidney. For example, the lipid based ligand binds HSA, or it binds HSA with a sufficient affinity such that the conjugate will be distributed to a non-kidney tissue but also be reversible. Alternatively, the lipid-based ligand binds HSA weakly or not at all, such that the conjugate will be distributed to the kidney. Other moieties that target to kidney cells can also be used in place of or in addition to the lipid-based ligand.

In another aspect, the ligand is a moiety, e.g., a vitamin, that is taken up by an embryonic cell, e.g., a proliferating cell. Exemplary vitamins include vitamin A, E, and K. Other exemplary vitamins include are B vitamin, e.g., folic acid, B12, riboflavin, biotin, pyridoxal or other vitamins or nutrients taken up by embryonic cells. Also included are HSA and low density lipoproteins.

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent can be an α-helical agent, and can include a lipophilic and a lipophobic phase.

The ligand can be a peptide or peptidomimetic. A peptidomimetic (also referred to herein as an oligopeptidomimetic) is a molecule capable of folding into a defined 3-dimensional structure similar to a natural peptide. The attachment of peptide and peptidomimetics to RNA effector molecule agents can affect pharmacokinetic distribution of the RNA effector molecule, such as by enhancing cellular recognition and absorption. The peptide or peptidomimetic moiety can be about 5 to 50 amino acids long, e.g., about 5, 10, 15, 20, 25, 30, 35, 40, 45, or 50 amino acids long (see Table 3, for example):

TABLE 3
Exemplary Cell Permeation Peptides

| Cell Permeation | | SEQ | |
|---|---|---|---|
| Peptide | Amino acid Sequence | ID NO: | Reference |
| Penetratin | RQIKIWFQNRRMKWKK | 3284943 | Derossi et al., 269 J. Biol. |
| | | | Chem. 10444 (1994) |
| Tat fragment | GRKKRRQRRRPPQC | 3284944 | Vives et al., 272 J. Biol. |
| (48-60) | | | Chem. 16010 (1997) |
| Signal Sequence- | GALFLGWLGAAGSTMGAWSQ | 3284945 | Chaloin et al., 243 Biochem. |
| based peptide | PKKKRKV | | Biophys. Res. Commun 601 |
| | | | (1998) |
| PVEC | LLIILRRRIRKQAHAHSK | 3284946 | Elmquist et al., 269 Exp. |
| | | | Cell Res. 237 (2001) |
| Transportan | GWTLNSAGYLLKINLKALAAL | 3284947 | Pooga et al., 12 FASEB |
| | AKKIL | | J. 67 (1998) |
| Amphiphilic | KLALKLALKALKAALKLA | 3284948 | Oehlke et al., 2 Mol. Ther. |
| model peptide | | | 339 (2000) |
| Arg₉ | RRRRRRRRR | 3284949 | Mitchell et al., 56 J. Pept. |
| | | | Res. 318 (2000) |
| Bacterial cell | KFFKFFKFFK | 3284950 | |
| wall permeating | | | |
| LL-37 | LLGDFFRKSKEKIGKEFKRIVQ | 3284951 | |
| | RIKDFLRNLVPRTES | | |
| Cecropin P1 | SWLSKTAKKLENSAKKRISEGI | 3284952 | |
| | AIAIQGGPR | | |
| α-defensin | ACYCRIPACIAGERRYGTCIYQ | 3284953 | |
| | GRLWAFCC | | |
| b-defensin | DHYNCVSSGGQCLYSACPIFTK | 3284954 | |
| | IQGTCYRGKAKCCK | | |
| Bactenecin | RKCRIVVIRVCR | 3284955 | |
| PR-39 | RRRPRPPYLPRPRPPPFFPPRLP | 3284956 | |
| | PRIPPGFPPRFPPRFPGKR-NH₂ | | |
| Indolicidin | ILPWKWPWWPWRR-NH₂ | 3284957 | |

A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO:3284958) An RFGF analogue (e.g., amino acid sequence AALLPVLLAAP (SEQ ID NO:3284959) containing a hydrophobic MTS can also be a targeting moiety. The peptide moiety can be a “delivery” peptide that carres large polar molecules including peptides, oligonucleotides, and protein across cell membranes. For example, sequences from the HIV Tat protein (GRKKRRQRRRPPQ (SEQ ID NO:3284960)) and the Drosophila antennapedia protein (RQIKIWFQNRRMKWKK (SEQ ID NO:284961) can function as delivery peptides. A peptide or peptidomimetic can be encoded by a random sequence of DNA, such as a peptide identified from a phage-display library, or one-bead-one-compound (OBOC) combinatorial library. Lam et al., 354 Nature 82-84 (1991). The peptide or peptidomimetic can be tethered to a dsRNA agent via an incorporated monomer unit is a cell targeting peptide such as an arginine-glycine-aspartic acid (RGD)-peptide, or RGD mimic. As noted, the peptide moieties can have a structural modification, such as to increase stability or direct conformational properties. Any of the structural modifications described herein can be utilized.

An RGD peptide moiety can be used to target a tumor cell, such as an endothelial tumor cell or a breast cancer tumor cell. Zitzmann et al., 62 Cancer Res. 5139-43 (2002). An RGD peptide can facilitate targeting of an dsRNA agent to tumors of a variety of other tissues, including the lung, kidney, spleen, or liver. Aoki et al., 8 Cancer Gene Ther. 783-87 (2001). Preferably, the RGD peptide will facilitate targeting of an RNA effector molecule agent to the kidney. The RGD peptide can be linear or cyclic, and can be modified, e.g., glycosylated or methylated to facilitate targeting to specific tissues. For example, a glycosylated RGD peptide can deliver a RNA effector molecule agent to a tumor cell expressing αVβ3. Haubner et al., 42 J. Nucl. Med. 326-36 (2001).

A “cell permeation peptide” is capable of permeating a cell, e.g., an avian cell. It can be, for example, an α-helical linear peptide (e.g., LL-37 or Ceropin P1), a disulfide bond-containing peptide (e.g., α-defensin, β-defensin or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen. Simeoni et al., 31 Nucl. Acids Res. 2717-24 (2003).

Representative patents that teach the preparation of oligonucleotide conjugates include, but are not limited to, U.S. Pat. No. 4,828,979; No. 4,948,882; No. 5,218,105; No. 5,525,465; No. 5,541,313; No. 5,545,730; No. 5,552,538; No. 5,578,717, No. 5,580,731; No. 5,591,584; No. 5,109,124; No. 5,118,802; No. 5,138,045; No. 5,414,077; No. 5,486,603; No. 5,512,439; No. 5,578,718; No. 5,608,046; No. 4,587,044; No. 4,605,735; No. 4,667,025; No. 4,762,779; No. 4,789,737; No. 4,824,941; No. 4,835,263; No. 4,876,335; No. 4,904,582; No. 4,958,013; No. 5,082,830; No. 5,112,963; No. 5,214,136; No. 5,082,830; No. 5,112,963; No. 5,214,136; No. 5,245,022; No. 5,254,469; No. 5,258,506; No. 5,262,536; No. 5,272,250; No. 5,292,873; No. 5,317,098; No. 5,371,241, No. 5,391,723; No. 5,416,203, No. 5,451,463; No. 5,510,475; No. 5,512,667; No. 5,514,785; No. 5,565,552; No. 5,567,810; No. 5,574,142; No. 5,585,481; No. 5,587,371; No. 5,595,726; No. 5,597,696; No. 5,599,923; No. 5,599,928; No. 5,688,941; No. 6,294,664; No. 6,320,017; No. 6,576,752; No. 6,783,931; No. 6,900,297; and No. 7,037,646.

It is not necessary for all positions in a given compound to be uniformly modified, and in fact more than one of the aforementioned modifications can be incorporated in a single compound or even at a single nucleoside within sn oligonucleotide. The present invention also includes oligonucleotide molecule compounds which are chimeric compounds. “Chimeric” RNA effector molecule compounds or “chimeras,” in the context of this invention, are oligonucleotide compounds, such as dsRNAs, that contain two or more chemically distinct regions, each made up of at least one monomer unit, i.e., a nucleotide in the case of a dsRNA compound. These RNA effector molecules typically contain at least one region wherein the RNA is modified so as to confer upon the RNA effector molecule increased resistance to nuclease degradation, increased cellular uptake, and/or increased binding affinity for the target nucleic acid. An additional region of the oligonucleotide can serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of RNA effector molecule inhibition of gene expression. Consequently, comparable results can often be obtained with shorter RNA effector molecules when chimeric dsRNAs are used, compared to phosphorothioate deoxydsRNAs hybridizing to the same target region. Cleavage of the oligonucleotide can be routinely detected by gel electrophoresis and, if necessary, associated nucleic acid hybridization techniques known in the art.

### VI. INTRODUCTION/DELIVERY OF RNA EFFECTOR MOLECULES

The delivery of an oligonucleotide (e.g., a RNA effector molecule) to cell within the embryonated egg according to methods provided herein can be achieved in a number of different ways. For example, delivery can be performed directly by administering a composition comprising a RNA effector molecule, e.g., a dsRNA, into an egg. Alternatively, delivery can be performed indirectly by administering into the egg one or more vectors that encode and direct the expression of the RNA effector molecule. These alternatives are discussed further herein.

In some embodiments, the RNA effector molecule is a siRNA or shRNA effector molecule introduced into a cell within the egg by introducing into the egg an invasive bacterium containing one or more siRNA or shRNA effector molecules or DNA encoding one or more siRNA or shRNA effector molecules (a process sometimes referred to as transkingdom RNAi (tkRNAi)). The invasive bacterium can be an attenuated strain of Listeria, Shigella, Salmonella, E. coli, or Bifidobacteriae, or a non-invasive bacterium that has been genetically modified to increase its invasive properties, e.g., by introducing one or more genes that enable invasive bacteria to access the cytoplasm of cells. Examples of such cytoplasm-targeting genes include listeriolysin O of Listeria and the invasin protein of Yersinia pseudotuberculosis. Methods for delivering RNA effector molecules to animal cells to induce transkingdom RNAi (tkRNAi) are known in the art. See, e.g., U.S. Patent Pubs. No. 2008/0311081 and No. 2009/0123426. In one embodiment, the RNA effector molecule is a siRNA molecule. In another embodiment, the RNA effector molecule is not a shRNA molecule.

As noted herein, oligonucleotides can be modified to prevent rapid degradation of the dsRNA by endo- and exo-nucleases and avoid undesirable off-target effects. For example, RNA effector molecules can be modified by chemical conjugation to lipophilic groups such as cholesterol to enhance cellular uptake and prevent degradation. In an alternative embodiment, the RNA effector molecule is not modified by chemical conjugation to a lipophilic group, e.g., cholesterol.

In an alternative embodiment, RNA effector molecules can be delivered using a drug delivery system such as a nanoparticle, a dendrimer, a polymer, a liposome, or a cationic delivery system. Positively charged cationic delivery systems facilitate binding of an RNA effector molecule (negatively charged) and also enhance interactions at the negatively charged cell membrane to permit efficient cellular uptake. Cationic lipids, dendrimers, or polymers can either be bound to RNA effector molecules, or induced to form a vesicle or micelle that encases the RNA effector molecule. See, e.g., Kim et al., 129 J. Contr. Release 107-16 (2008). Methods for making and using cationic-RNA effector molecule complexes are well within the abilities of those skilled in the art. See e.g., Sorensen et al 327 J. Mol. Biol. 761-66 (2003); Verma et al., 9 Clin. Cancer Res. 1291-1300 (2003); Arnold et al., 25 J. Hypertens. 197-205 (2007).

Where the RNA effector molecule is a double-stranded molecule, such as a small interfering RNA (siRNA), comprising a sense strand and an antisense strand, the sense strand and antisense strand can be separately and temporally exposed. The phrase “separately and temporally” refers to the introduction of each strand of a double-stranded RNA effector molecule to an egg in a single-stranded form, e.g., in the form of a non-annealed mixture of both strands or as separate, i.e., unmixed, preparations of each strand. In some embodiments, there is a time interval between the introduction of each strand which can range from seconds to several minutes to about an hour or more, e.g., 12 hr, 24 hr, 48 hr, 72 hr, 84 hr, 96 hr, or 108 hr, or more. Separate and temporal administration can be performed with canonical or non-canonical RNA effector molecules.

It is also contemplated herein that a plurality of RNA effector molecules are administered in a separate and temporal manner Thus, each of a plurality of RNA effector molecules can be administered at a separate time or at a different frequency interval to achieve the desired average percent inhibition for the target gene. For example, RNA effector molecules targeting Bak can be administered more frequently than RNA effector molecule targeting LDH, as the expression of Bak recovers faster following treatment with a Bak RNA effector molecule. In one embodiment, the RNA effector molecules are added at a concentration from approximately 0.01 nM to 200 nM, as such concentrations are calculated based on the capacity of the egg. In another embodiment, the RNA effector molecules are added at an amount of approximately 50 molecules per cell up to and including 500,000 molecules per cell. In another embodiment, the RNA effector molecules are added at a concentration from about 0.1 fmol/10⁶ cells to about 1 pmol/10⁶ cells, as calculated based on the capacity of the egg.

In another aspect, a RNA effector molecule for modulating expression of a target gene can be expressed from transcription units inserted into DNA or RNA vectors. See, e.g., Couture et al., 12 TIG 5-10 (1996); WO 00/22113; WO 00/22114; U.S. Pat. No. 6,054,299. Expression can be transient (on the order of hours to days) or sustained (for several days to about a week), depending upon the specific construct used and the target tissue in the egg. These transgenes can be introduced as a linear construct, a circular plasmid, or a viral vector, which can be an integrating or non-integrating vector. The transgene can also be constructed to permit it to be inherited as an extra chromosomal plasmid. Gassmann, et al., 92 PNAS 1292 (1995).

The individual strand or strands of a RNA effector molecule can be transcribed from a promoter on an expression vector. Where two separate strands are to be expressed to generate, for example, a dsRNA, two separate expression vectors can be co-introduced (e.g., by transfection or infection) into a target cell. Alternatively each individual strand of a dsRNA can be transcribed by promoters both of which are located on the same expression plasmid. In one embodiment, a dsRNA is expressed as an inverted repeat joined by a linker polynucleotide sequence such that the dsRNA has a stem and loop structure.

RNA effector molecule expression vectors are generally DNA plasmids or viral vectors. Expression vectors compatible with avian cells can be used to produce recombinant constructs for the expression of an RNA effector molecule as described herein. Eukaryotic cell expression vectors are well known in the art and are available from a number of commercial sources. Typically, such vectors are provided containing convenient restriction sites for insertion of the desired nucleic acid segment. RNA effector molecule expressing vectors can be delivered directly to target cells using standard transfection and transduction methods.

RNA effector molecule expression plasmids can be transfected into an egg a complex with cationic lipid carriers (e.g., OLIGOFECTAMINE™ reagent) or non-cationic lipid-based carriers (e.g., TRANSIT-TKO® transfection reagent, Mirus Bio LLC, Madison, Wis.). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as Green Fluorescent Protein (GFP). Stable transfection of cells ex vivo can be ensured using markers that provide the transfected cell with resistance to specific environmental factors (e.g., antibiotics and drugs), such as hygromycin B resistance. RNA effector molecule expression plasmids can be transfected into target cells as a complex with cationic lipid carriers (e.g., OLIGOFECTAMINE™ reagent) or non-cationic lipid-based carriers (e.g., TRANSIT-TKO® transfection reagent). Multiple lipid transfections for RNA effector molecule-mediated knockdowns targeting different regions of a target RNA over a period of a week or more are also contemplated by the invention. Successful introduction of vectors into host cells can be monitored using various known methods. For example, transient transfection can be signaled with a reporter, such as a fluorescent marker, such as GFP. Stable transfection of cells ex vivo can be ensured using markers that provide the transfected cell with resistance to specific environmental factors (e.g., antibiotics and drugs), such as hygromycin B resistance.

Viral vector systems that can be utilized with the methods and compositions described herein include, but are not limited to, (a) adenovirus vectors; (b) retrovirus vectors, including but not limited to lentiviral vectors, moloney murine leukemia virus, etc.; (c) adeno-associated virus vectors; (d) herpes simplex virus vectors; (e) SV 40 vectors; (f) polyoma virus vectors; (g) papilloma virus vectors; (h) picornavirus vectors; (i) pox virus vectors such as an orthopox, e.g., vaccinia virus vectors or avipox, e.g., canary pox or fowl pox; and (j) a helper-dependent or gutless adenovirus. Replication-defective viruses can also be advantageous. Different vectors will or will not become incorporated into the cells' genome. The constructs can include viral sequences for transfection, if desired. Alternatively, the construct can be incorporated into vectors capable of episomal replication, e.g., EPV and EBV vectors. Constructs for the recombinant expression of an RNA effector molecule will generally require regulatory elements, e.g., promoters, enhancers, etc., to ensure the expression of the RNA effector molecule in target cells. Other aspects to consider for vectors and constructs are further described herein.

Vectors useful for the delivery of a RNA effector molecule will include regulatory elements (promoter, enhancer, etc.) sufficient for expression of the RNA effector molecule in the desired target cell or tissue. The regulatory elements can be chosen to provide either constitutive or regulated/inducible expression.

Expression of the RNA effector molecule can be regulated, for example, by using an inducible regulatory sequence that is sensitive to certain physiological regulators, e.g., glucose levels. Docherty et al., 8 FASEB J. 20-24 (1994). Such inducible expression systems, suitable for the control of dsRNA expression in cells include, for example, regulation by ecdysone, estrogen, progesterone, tetracycline, chemical inducers of dimerization, and isopropyl-(3-D1-thiogalactopyranoside (IPTG). A person skilled in the art can choose the appropriate regulatory/promoter sequence based on the intended use of the RNA effector molecule transgene.

In a specific embodiment, viral vectors that contain nucleic acid sequences encoding a RNA effector molecule can be used. For example, a retroviral vector can be used. See Miller et al., 217 Meth. Enzymol. 581-99 (1993); U.S. Pat. No. 6,949,242. Retroviral vectors contain the components necessary for the correct packaging of the viral genome and integration into the host cell DNA. The nucleic acid sequences encoding an RNA effector molecule are cloned into one or more vectors, which facilitates delivery of the nucleic acid into a cell. More detail about retroviral vectors can be found, for example, in Boesen et al., 6 Biotherapy 291-302 (1994), which describes the use of a retroviral vector to deliver the mdr1 gene to hematopoietic stem cells in order to make the stem cells more resistant to chemotherapy. Other references illustrating the use of retroviral vectors in gene therapy include Clowes et al., 93J. Clin. Invest. 644-651 (1994); Kiem et al., 83 Blood 1467-73 (1994); Salmons & Gunzberg, 4 Human Gene Ther. 129-11 (1993); Grossman & Wilson, 3 Curr. Opin. Genetics Devel. 110-14 (1993). Lentiviral vectors contemplated for use include, for example, the HIV based vectors described in U.S. Pat. No. 6,143,520; No. 5,665,557; and No. 5,981,276.

It should be noted that host cell-surface receptors for retroviral entry can be inhabited by ERV Env proteins (virus interference). See Miller, 93 PNAS 11407-13 (1996). The retroviral envelope (Env) protein mediates the binding of virus particles to their cellular receptors, enabling virus entry: the first step in a new replication cycle. If an ERV is expressed in a cell, re-infection by a related exogenous retrovirus is prevented through interference (also called receptor interference): the Env protein of an ERV that is inserted into the cell membrane will interfere with the corresponding exogenous virus by receptor competition. This protects the cell from being overloaded with retroviruses. For example, enJSRVs can block the entry of exogenous JSRVs because they all utilize the cellular hyaluronidase-2 as a receptor. Spencer et al., 77 J. Virol. 5749-53 (2003). It is noteworthy that defective ERVs are no less interfering. Two enJSRVs, enJS56A1 and enJSRV-20, contain a mutant gag polyprotein that can interfere with the late stage replication of exogenous JSRVs. Arnaud et al., 2 PLoS e170 (2007). Thus, interference between defective and replication-competent retroviruses provides an important mechanism of ERV copy number control. Receptor interference by ERV-expressed Env molecules (e.g., expressed by the HERV-H family) can hinder transfection or re-infection of cells intended to produce recombinant proteins. Such effects can explain low copy number or low expression in retroviral vector-mediated recombinant host cells, such as host cells transfected with two retroviral vectors, each encoding a single, complementary antibody chain. Hence, a target gene of the present embodiments that inhibits expression of ERV Env protein(s) provides for increasing retroviral vector multiplicity in the egg's cells and increased yield of biological product.

Adenoviruses are also contemplated for use in delivery of RNA effector molecules. A suitable AV vector for expressing an RNA effector molecule featured in the invention, a method for constructing the recombinant AV vector, and a method for delivering the vector into target cells, are described in Xia et al., 20 Nature Biotech. 1006-10 (2002).

Use of Adeno-associated virus (AAV) vectors is also contemplated (Walsh et al., 204 Proc. Soc. Exp. Biol. Med. 289-300 (1993); U.S. Pat. No. 5,436,146. In one embodiment, the RNA effector molecule can be expressed as two separate, complementary single-stranded RNA molecules from a recombinant AAV vector having, for example, either the U6 or H1 RNA promoters, or the cytomegalovirus (CMV) promoter. Suitable AAV vectors for expressing the dsRNA featured in the invention, methods for constructing the recombinant AV vector, and methods for delivering the vectors into target cells are described in Samulski et al., 61J. Virol. 3096-101 (1987); Fisher et al., 70 J. Virol, 70: 520-32 (1996); Samulski et al., 63 J. Virol. 3822-26 (1989); U.S. Pat. No. 5,252,479 and No. 5,139,941; WO 94/13788; WO 93/24641.

Another viral vector useful in egg-based bioprocessing is a pox virus such as a vaccinia virus, for example an attenuated vaccinia such as Modified Virus Ankara (MVA) or NYVAC, an avipox such as fowl pox or canary pox.

The tropism of viral vectors can be modified by pseudotyping the vectors with envelope proteins or other surface antigens from other viruses, or by substituting different viral capsid proteins, as appropriate. For example, lentiviral vectors can be pseudotyped with surface proteins from vesicular stomatitis virus (VSV), rabies, Ebola, Mokola, Baculovirus, and the like. Mononegavirales, e.g., VSV or respiratory syncytial virus (RSV) can be pseudotyped with Baculovirus. U.S. Pat. No. 7,041,489. AAV vectors can be made to target different cells by engineering the vectors to express different capsid protein serotypes. See, e.g., Rabinowitz et al., 76 J. Virol. 791-801 (2002).

In one embodiment, the invention provides compositions containing a RNA effector molecule, as described herein, and an acceptable carrier. The composition containing the RNA effector molecule is useful for enhancing the production of a biological product by an egg by modulating the expression or activity of a target gene in the egg's cells. Such compositions are formulated based on the mode of delivery. Provided herein are exemplary RNA effector molecules useful in improving the production of a biological product, such as an immunogenic agent. In one embodiment, the RNA effector molecule in the composition is a siRNA. Alternatively, the RNA effector molecule in the composition is not a siRNA.

In another embodiment, a composition is provided herein comprising a plurality of RNA effector molecules that permit inhibition of expression of an immune response pathway and a cellular process; such as INFAR1, IRF3, MAVS, PKR, or IFITM1 genes, and PTEN, BAK, CDKNA2, FN1, or LDHA genes. The composition can optionally be combined (or administered) with at least one additional RNA effector molecule targeting an additional cellular process including, but not limited to: carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of pH, and protein production.

In one embodiment, the compositions described herein comprise a plurality of RNA effector molecules. In one embodiment of this aspect, each of the plurality of RNA effector molecules is provided at a different concentration. In another embodiment of this aspect, each of the plurality of RNA effector molecules is provided at the same concentration. In another embodiment of this aspect, at least two of the plurality of RNA effector molecules are provided at the same concentration, while at least one other RNA effector molecule in the plurality is provided at a different concentration. It is appreciated one of skill in the art that a variety of combinations of RNA effector molecules and concentrations can be provided to a cell in an embryonated egg to produce the desired effects described herein.

The compositions featured herein are administered in amounts sufficient to inhibit expression of target genes. In general, a suitable dose of RNA effector molecule will be in the range of 0.001 to 200.0 milligrams per unit volume per day. In another embodiment, the RNA effector molecule is provided in the range of 0.001 nM to 200 mM per day, generally in the range of 0.1 nM to 500 nM, inclusive. For example, the dsRNA can be administered at 0.01 nM, 0.05 nM, 0.1 nM, 0.5 nM, 0.75 nM, 1 nM, 1.5 nM, 2 nM, 3 nM, 10 nM, 20 nM, 30 nM, 40 nM, 50 nM, 100 nM, 200 nM, 400 nM, or 500 nM per single dose.

The composition can be administered once daily, or the RNA effector molecule can be administered as two, three, or more sub-doses at appropriate intervals throughout the day or delivery through a controlled release formulation. In that case, the RNA effector molecule contained in each sub-dose must be correspondingly smaller in order to achieve the total daily dosage. The dosage unit can also be compounded for delivery over several days, e.g., using a conventional sustained release formulation, which provides sustained release of the RNA effector molecule over a several-day-period. Sustained release formulations are well known in the art and are particularly useful for delivery of agents to a particular site, such as could be used with the agents of the present invention. It should be noted that when administering a plurality of RNA effector molecules, one should consider that the total dose of RNA effector molecules will be higher than when each is administered alone. For example, administration of three RNA effector molecules each at 1 nM (e.g., for effective inhibition of target gene expression) will necessarily result in a total dose of 3 nM to the cell. One of skill in the art can modify the necessary amount of each RNA effector molecule to produce effective inhibition of each target gene while preventing any unwanted toxic effects to the embryo resulting from high concentrations of either the RNA effector molecules or delivery agent.

The effect of a single dose on target gene transcript levels can be long-lasting, such that subsequent doses are administered at not more than 3-, 4-, or 5-day intervals, or at not more than 1-, 2-, 3-, or 4-week intervals.

In one embodiment, the administration of the RNA effector molecule is ceased at least 6 hr, at least 12 hr, at least 18 hr, at least 36 hr, at least 48 hr, at least 60 hr, at least 72 hr, at least 96 hr, or at least 120 hr, or at least 1 week, before isolation of the biological product. Thus in one embodiment, contacting a host cell (e.g., in an embryonated egg) with a RNA effector molecule is complete at least 6 hr, at least 12 hr, at least 18 hr, at least 36 hr, at least 48 hr, at least 60 hr, at least 72 hr, at least 96 hr, or at least 120 hr, or at least 1 week, before isolation of the biological product.

It is also noted that, in certain embodiments, it can be beneficial to contact the egg cells with an RNA effector molecule such that a constant number (or at least a minimum number) of RNA effector molecules per egg is maintained. Maintaining the levels of the RNA effector molecule as such can ensure that modulation of target gene expression is maintained even at high cell densities. This can be accomplished using, for example, controlled release polymers, as are well-known in the art.

Alternatively, the amount of an RNA effector molecule can be administered according to the cell density. In such embodiments, the RNA effector molecule(s) is added at a concentration of at least 0.01 fmol/10⁶ cells, at least 0.1 fmol/10⁶ cells, at least 0.5 fmol/10⁶ cells, at least 0.75 fmol/10⁶ cells, at least 1 fmol/10⁶ cells, at least 2 fmol/10⁶ cells, at least 5 fmol/10⁶ cells, at least 10 fmol/10⁶ cells, at least 20 fmol/10⁶ cells, at least 30 fmol/10⁶ cells, at least 40 fmol/10⁶ cells, at least 50 fmol/10⁶ cells, at least 60 fmol/10⁶ cells, at least 100 fmol/10⁶ cells, at least 200 fmol/10⁶ cells, at least 300 fmol/10⁶ cells, at least 400 fmol/10⁶ cells, at least 500 fmol/10⁶ cells, at least 700 fmol/10⁶ cells, at least 800 fmol/10⁶ cells, at least 900 fmol/10⁶ cells, or at least 1 pmol/10⁶ cells, or more, based on the age and capacity of the egg.

In an alternate embodiment, the RNA effector molecule is administered at a dose of at least 10 molecules per cell, at least 20 molecules per cell (molecules/cell), at least 30 molecules/cell, at least 40 molecules/cell, at least 50 molecules/cell, at least 60 molecules/cell, at least 70 molecules/cell, at least 80 molecules/cell, at least 90 molecules/cell at least 100 molecules/cell, at least 200 molecules/cell, at least 300 molecules/cell, at least 400 molecules/cell, at least 500 molecules/cell, at least 600 molecules/cell, at least 700 molecules/cell, at least 800 molecules/cell, at least 900 molecules/cell, at least 1000 molecules/cell, at least 2000 molecules/cell, at least 5000 molecules/cell or more, inclusive.

In some embodiments, the RNA effector molecule is administered at a dose within the range of 10-100 molecules/cell, 10-90 molecules/cell, 10-80 molecules/cell, 10-70 molecules/cell, 10-60 molecules/cell, 10-50 molecules/cell, 10-40 molecules/cell, 10-30 molecules/cell, 10-20 molecules/cell, 90-100 molecules/cell, 80-100 molecules/cell, 70-100 molecules/cell, 60-100 molecules/cell, 50-100 molecules/cell, 40-100 molecules/cell, 30-100 molecules/cell, 20-100 molecules/cell, 30-60 molecules/cell, 30-50 molecules/cell, 40-50 molecules/cell, 40-60 molecules/cell, or any range there between.

In one embodiment of the methods described herein, the RNA effector molecule is provided to the eggs in a continuous infusion. The continuous infusion can be initiated at day zero (e.g., the first day of culture or day of inoculation with an RNA effector molecule) or can be initiated at any time period during the biological production process. Similarly, the continuous infusion can be stopped at any time point during the biological production process. Thus, the infusion of a RNA effector molecule or composition can be provided and/or removed at a particular phase of embryo development or viral replication, a window of time in the production process, or at any other desired time point. The continuous infusion can also be provided to achieve a “desired average percent inhibition” for a target gene.

In one embodiment, a continuous infusion can be used following an initial bolus administration of an RNA effector molecule to an egg. In this embodiment, the continuous infusion maintains the concentration of RNA effector molecule above a minimum level over a desired period of time. The continuous infusion can be delivered at a rate of 0.03 pmol/L of egg/hour to 3 pmol/L of culture/hour, for example, at 0.03 pmol/L/hr, 0.05 pmol/L/hr, 0.08 pmol/L/hr, 0.1 pmol/L/hr, 0.2 pmol/L/hr, 0.3 pmol/L/hr, 0.5 pmol/L/hr, 1.0 pmol/L/hr, 2 pmol/L/hr, or 3 pmol/L/hr, or any value there between.

In one embodiment, the RNA effector molecule is administered as a sterile aqueous solution. In one embodiment, the RNA effector molecule is formulated in a non-lipid formulation. In another embodiment, the RNA effector molecule is formulated in a cationic or non-cationic lipid formulation. In still another embodiment, the RNA effector molecule is formulated in a medium suitable for introduction into an egg. In another embodiment, the RNA effector molecule is administered to the egg at a particular stage of cell growth or embryo development.

The RNA effector molecule(s) can be administered once daily, or the RNA effector molecule treatment can be repeated (e.g., two, three, or more doses) by adding the composition to the culture medium at appropriate intervals/frequencies throughout the production of the biological product. As used herein the term “frequency” refers to the interval at which transfection of the cell culture occurs and can be optimized by one of skill in the art to maintain the desired level of inhibition for each target gene. In one embodiment, RNA effector molecules are contacted with cells at a frequency of every 48 hours. In other embodiments, the RNA effector molecules are administered at a frequency of e.g., every 4 hr, every 6 hr, every 12 hr, every 18 hr, every 24 hr, every 36 hr, every 72 hr, every 84 hr, every 96 hr, every 5 days, every 7 days, every 10 days, every 14 days, every 3 weeks, or more during the production of the biological product. The frequency can also vary, such that the interval between each dose is different (e.g., first interval 36 hr; second interval 48 hr; third interval 72 hr, etc).

The frequency of RNA effector molecule treatment can be optimized to maintain an “average percent inhibition” of a particular target gene. As used herein, the term “desired average percent inhibition” refers to the average degree of inhibition of target gene expression over time that is necessary to produce the desired effect and which is below the degree of inhibition that produces any unwanted or negative effects. For example, the desired inhibition of Bax/Bak is typically >80% inhibition to effect a decrease in apoptosis, while the desired average inhibition of LDH can be less (e.g., 70%) as high degrees of LDH average inhibition (e.g., 90%) decrease cell viability. In some embodiments, the desired average percent inhibition is at least 20%, at least 30%, at least 40%, at least 50%, at least 60%, at least 70%, at least 80%, at least 85%, at least 90%, at least 95%, at least 99%, or even 100% (i.e., absent). One of skill in the art can use routine cell death assays to determine the upper limit for desired percent inhibition (e.g., level of inhibition that produces unwanted effects). Determination of LD₅₀ in eggs is known in the art, see e.g., Banks et al. 1 Infect. Immun 259-62 (1970). One of skill in the art can also use methods to detect target gene expression (e.g., PERT) to determine an amount of an RNA effector molecule that produces gene modulation. See Zhang et al., 102 Biotech. Bioeng. 1438-47 (2009). The percent inhibition is described herein as an average value over time, since the amount of inhibition is dynamic and can fluctuate slightly between doses of the RNA effector molecule.

The compositions of the present invention can be formulated as suspensions in aqueous, non-aqueous or mixed media. Aqueous suspensions can further contain substances which increase the viscosity of the suspension including, for example, sodium carboxymethylcellulose, sorbitol and/or dextran. The suspension can also contain stabilizers.

In one embodiment, the composition comprising a RNA effector molecule further comprises one or more supplements. Example supplements include, but are not limited to, essential amino acids (e.g., glutamine), 2-mercapto-ethanol, bovine serum albumin (BSA), lipid concentrate, cholesterol, catalase, insulin, human transferrin, superoxide dismutase, biotin, DL α-tocopherol acetate, DL α-tocopherol, vitamins (e.g., Vitamin A (acetate), choline chloride, D calcium pantothenate, folic acid, nicotinamide, pyridoxal hydrochloride, riboflavin, thiamine hydrochloride, i-Inositol), corticosterone, D-galactose, ethanolamine HCl, glutathione (reduced), L-carnitine HCl, linoleic acid, linolenic acid, progesterone, putrescine 2HCl, sodium selenite, T3 (triodo-I-thyronine), growth factors (e.g., EGF), iron, L-glutamine, L-alanyl-L-glutamine, sodium hypoxanthine, aminopterin and thymidine, arachidonic acid, acetate, ethyl alcohol 100%, myristic acid, oleic acid, palmitic acid, palmitoleic acid, PLURONIC F68® (Invitrogen, Carlsbad, Calif. 92008), stearic acid 10, TWEEN 80® nonionic surfactant (Invitrogen, Carlsbad, Calif.), sodium pyruvate, and glucose.

Lipid/Oligonucleotide Complexes

In some embodiments, a reagent that facilitates RNA effector molecule cellular uptake comprises a charged lipid, an emulsion, a liposome, a cationic or non-cationic lipid, an anionic lipid, a transfection reagent or a penetration enhancer as described herein. In one embodiment, the reagent that facilitates RNA effector molecule uptake comprises a charged lipid as described in U.S. Application Ser. No. 61/267,419, filed 7 Dec. 2009, and U.S. Application Ser. No. 61/334,398, filed 13 Can 2010.

The RNA effector molecules of the present invention can be encapsulated within liposomes or can form complexes thereto, in particular to cationic liposomes. Alternatively, RNA effector molecules can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride, or acceptable salts thereof.

In one embodiment, the RNA effector molecules are fully encapsulated in the lipid formulation (e.g., to form a SPLP, pSPLP, SNALP, or other nucleic acid-lipid particle). The term “SNALP” refers to a stable nucleic acid-lipid particle: a vesicle of lipids coating a reduced aqueous interior comprising a nucleic acid such as an RNA effector molecule or a plasmid from which an RNA effector molecule is transcribed. SNALPs are described, e.g., in U.S. Patent Pubs. No. 2006/0240093, No. 2007/0135372; No. 2009/0291131; U.S. patent application Ser. No. 12/343,342; No. 12/424,367. The term “SPLP” refers to a nucleic acid-lipid particle comprising plasmid DNA encapsulated within a lipid vesicle. SNALPs and SPLPs typically contain a cationic lipid, a non-cationic lipid, and a lipid that prevents aggregation of the particle (e.g., a PEG-lipid conjugate). SPLPs include “pSPLP,” which include an encapsulated condensing agent-nucleic acid complex as set forth in WO 00/03683. The particles in this embodiment typically have a mean diameter of about 50 nm to about 150 nm, or about 60 nm to about 130 nm, or about 70 nm to about 110 nm, or typically about 70 nm to about 90 nm, inclusive, and are substantially nontoxic. In addition, the nucleic acids when present in the nucleic acid-lipid particles of the present invention are resistant in aqueous solution to degradation with a nuclease. Nucleic acid-lipid particles and their method of preparation are reported in, e.g., U.S. Pat. No. 5,976,567; No. 5,981,501; No. 6,534,484; No. 6,586,410; No. 6,815,432; and WO 96/40964.

The lipid to drug ratio (mass/mass ratio) (e.g., lipid to dsRNA ratio) can be in ranges of from about 1:1 to about 50:1, from about 1:1 to about 25:1, from about 3:1 to about 15:1, from about 4:1 to about 10:1, from about 5:1 to about 9:1, or about 6:1 to about 9:1, inclusive.

A cationic lipid of the formulation can comprise at least one protonatable group having a pKa of from 4 to 15. The cationic lipid can be, for example, N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(1-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.C1), 1,2-Dilinoleoyl-3-trimethylaminopropane chloride salt (DLin-TAP.C1), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 2,2-Dilinoleyl-4-dimethylaminomethyl-[1,3]-dioxolane (DLin-K-DMA), 2,2-Dilinoleyl-4-dimethylaminoethyl-[1,3]-dioxolane, or a mixture thereof. The cationic lipid can comprise from about 20 mol % to about 70 mol %, inclusive, or about 40 mol % to about 60 mol %, inclusive, of the total lipid present in the particle. In one embodiment, cationic lipid can be further conjugated to a ligand.

A non-cationic lipid can be an anionic lipid or a neutral lipid, such as distearoyl-phosphatidylcholine (DSPC), dioleoylphosphatidylcholine (DOPC), dipalmitoyl-phosphatidylcholine (DPPC), dioleoylphosphatidylglycerol (DOPG), dipalmitoyl-phosphatidylglycerol (DPPG), dioleoyl-phosphatidylethanolamine (DOPE), palmitoyloleoyl-phosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE),16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be from about 5 mol % to about 90 mol %, inclusive, of about 10 mol %, to about 58 mol %, inclusive, if cholesterol is included, of the total lipid present in the particle.

The lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA can be, for example, a PEG-dilauryloxypropyl (C12), a PEG-dimyristyloxypropyl (C14), a PEG-dipalmityloxypropyl (C16), or a PEG-distearyloxypropyl (C18). The lipid that prevents aggregation of particles can be from 0 mol % to about 20 mol % or about 2 mol % of the total lipid present in the particle. In one embodiment, PEG lipid can be further conjugated to a ligand.

In some embodiments, the nucleic acid-lipid particle further includes a steroid such as, cholesterol at, e.g., about 10 mol % to about 60 mol %, inclusive, or about 48 mol % of the total lipid present in the particle.

In one embodiment, the lipid particle comprises a steroid, a PEG lipid and a cationic lipid of formula (I):

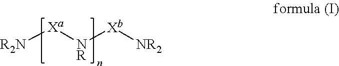

wherein each Xa and Xb, for each occurrence, is independently C₁₋₆ alkylene;

n is 0, 1, 2, 3, 4, or 5; each R is independently H,

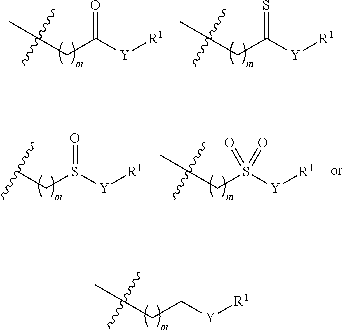

m is 0, 1, 2, 3 or 4; Y is absent, O, NR², or S; R¹ is alkyl alkenyl or alkynyl; each of which is optionally substituted with one or more substituents; and R² is H, alkyl alkenyl or alkynyl; each of which is optionally substituted each of which is optionally substituted with one or more substituents.

In one example, the lipidoid ND98.4HCl (MW 1487) (Formula 2), Cholesterol (Sigma-Aldrich), and PEG-Ceramide C16 (Avanti Polar Lipids) can be used to prepare lipid RNA effector molecule nanoparticles (e.g., LNP01 particles). Stock solutions of each in ethanol can be prepared as follows: ND98, 133 mg/mL; Cholesterol, 25 mg/mL, PEG-Ceramide C16, 100 mg/mL. The ND98, Cholesterol, and PEG-Ceramide C16 stock solutions can then be combined in a, e.g., 42:48:10 molar ratio. The combined lipid solution can be mixed with aqueous RNA effector molecule (e.g., in sodium acetate pH 5) such that the final ethanol concentration is about 35% to 45% and the final sodium acetate concentration is about 100 mM to 300 mM, inclusive. Lipid RNA effector molecule nanoparticles typically form spontaneously upon mixing. Depending on the desired particle size distribution, the resultant nanoparticle mixture can be extruded through a polycarbonate membrane (e.g., 100 nm cut-off) using, for example, a thermobarrel extruder, such as Lipex Extruder (Northern Lipids, Inc). In some cases, the extrusion step can be omitted. Ethanol removal and simultaneous buffer exchange can be accomplished by, for example, dialysis or tangential flow filtration. Buffer can be exchanged with, for example, phosphate buffered saline (PBS) at about pH 7, e.g., about pH 6.9, about pH 7.0, about pH 7.1, about pH 7.2, about pH 7.3, or about pH 7.4.

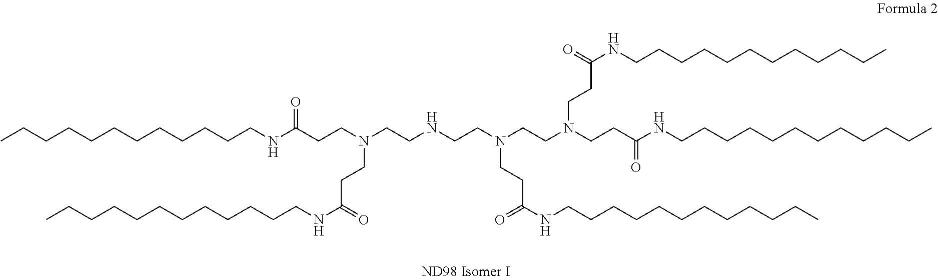 [SMILES: CCCCCCCCCCCCNC(=O)CCN(CCNCCN(CCC(=O)NCCCCCCCCCCCC)CCN(CCC(=O)NCCCCCCCCCCCC)CCC(=O)NCCCCCCCCCCCC)CCC(=O)NCCCCCCCCCCCC]

LNP01 formulations are described elsewhere, e.g., WO 2008/042973.

In certain embodiments, a lipid formulation is used in a RNA effector molecule composition as a reagent that facilitates RNA effector molecule uptake. In certain embodiments, the lipid formulation can be a LNP formulation, a LNP01 formulation, a XTC-SNALP formulation, or a SNALP formulation as described herein. In related embodiments, the XTC-SNALP formulation is as follows: using 2,2-Dilinoleyl-4-dimethylaminoethyl-[1,3]-dioxolane (XTC) with XTC/DPPC/Cholesterol/PEG-cDMA in a ratio of 57.1/7.1/34.4/1.4 and a lipid:siRNA ratio of about 7. In still other related embodiments, the RNA effector molecule is a dsRNA and is formulated in a XTC-SNALP formulation as follows: using 2,2-Dilinoleyl-4-dimethylaminoethyl-[1,3]-dioxolane (XTC) with a XTC/DPPC/Cholesterol/PEG-cDMA in a ratio of 57.1/7.1/34.4/1.4 and a lipid:siRNA ratio of about 7.

Alternatively, a RNA effector molecule such as those described herein can be formulated in a LNP09 formulation as follows: using XTC/DSPC/Chol/PEG2000-C14 in a ratio of 50/10/38.5/1.5 mol % and a lipid:siRNA ratio of about 11:1. In some embodiments, the RNA effector molecule is formulated in a LNP11 formulation as follows: using MC3/DSPC/Chol/PEG2000-C14 in a ratio of 50/10/38.5/1.5 mol % and a lipid:siRNA ratio of about 11:1. In still another embodiment, the RNA effector molecule is formulated in a LNP09 formulation or a LNP11 formulation and reduces the target gene mRNA levels by about 85% to 90% at a dose of 0.3 mg/kg, relative to a PBS control group. In yet another embodiment, the RNA effector molecule is formulated in a LNP09 formulation or a LNP 11 formulation and reduces the target gene mRNA levels by about 50% at a dose of 0.1 mg/kg, relative to a PBS control group. In yet another embodiment, the RNA effector molecule is formulated in a LNP09 formulation or a LNP 11 formulation and reduces the target gene protein levels in a dose-dependent manner relative to a PBS control group as measured by a western blot. In yet another embodiment, the RNA effector molecule is formulated in a SNALP formulation as follows: using D1 in DMA with a DLinDMA/DPPC/Cholesterol/PEG2000-cDMA in a ratio of 57.1/7.1/34.4/1.4 and a lipid:siRNA ratio of about 7.

In some embodiments, the lipid formulation comprises a lipid having the following formula:

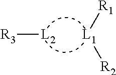

where R₁ and R₂ are each independently for each occurrence optionally substituted C₁₀-C₃₀ alkyl, optionally substituted C₁₀-C₃₀ alkoxy, optionally substituted C₁₀-C₃₀ alkenyl, optionally substituted C₁₀-C₃₀ alkenyloxy, optionally substituted C₁₀-C₃₀ alkynyl, optionally substituted C₁₀-C₃₀ alkynyloxy, or optionally substituted C₁₀-C₃₀ acyl;

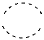

represents a connection between L₂ and L₁ which is:

(1) a single bond between one atom of L₂ and one atom of L₁, wherein
L₁ is C(Rₓ), O, S or N(O);
L₂ is —CR₅R₆—, —O—, —S—, —N(Q)-, ═C(R₅)—, —C(O)N(Q)-, —C(O)O—, —N(Q)C(O)—, —OC(O)—, or —C(O)—;
(2) a double bond between one atom of L₂ and one atom of L₁; wherein
L₁ is C;
   L₂ is —CR₅═, —N(Q)=, —N—, —O—N═, —N(Q)-N═, or —C(O)N(Q)-N═;
(3) a single bond between a first atom of L₂ and a first atom of L₁, and a single bond between a second atom of L₂ and the first atom of L₁, wherein
   L₁ is C;
   L₂ has the formula

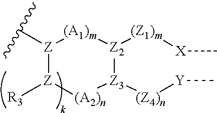

wherein
X is the first atom of L₂, Y is the second atom of L₂, - - - - - represents a single bond to the first atom of L₁, and X and Y are each, independently, selected from the group consisting of —O—, —S—, alkylene, —N(Q)-, —C(O)—, —O(CO)—, —OC(O)N(Q)-, —N(O)C(O)O—, —C(O)O, —OC(O)O—, —OS(O)(Q₂)O—, and —OP(O)(Q₂)O—;
Z₁ and Z₄ are each, independently, —O—, —S—, —CH₂—, —CHR⁵—, or —CR⁵R⁵—;
Z₂ is CH or N;
Z₃ is CH or N;
or Z₂ and Z₃, taken together, are a single C atom;
A₁ and A₂ are each, independently, —O—, —S—, —CH₂—, —CR⁵R⁵—, or —CR⁵R⁵—;
each Z is N, C(R₅), or C(R₃);
k is 0, 1, or 2;
each m, independently, is 0 to 5;
each n, independently, is 0 to 5;

where m and n taken together result in a 3, 4, 5, 6, 7 or 8 member ring;

(4) a single bond between a first atom of L₂ and a first atom of L₁, and a single bond between the first atom of L₂ and a second atom of L₁, wherein

(A) L₁ has the formula:

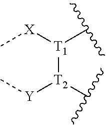

wherein
X is the first atom of L₁, Y is the second atom of L₁, - - - - - represents a single bond to the first atom of L₂, and X and Y are each, independently, selected from the group consisting of —O—, —S—, alkylene, —N(O)—, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is CH or N;
T₂ is CH or N;
or T₁ and T₂ taken together are C═C;
L₂ is CR₅; or

(B) L₁ has the formula:

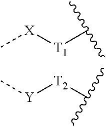

wherein

X is the first atom of L₁, Y is the second atom of L₁, - - - - - represents a single bond to the first atom of L₂, and X and Y are each, independently, selected from the group consisting of —O—, —S—, alkylene, —N(O)—, —C(O)—, —O(CO)—, —OC(O)N(Q)-, —N(Q)C(O)O—, —C(O)O, —OC(O)O—, —OS(O)(Q₂)O—, and —OP(O)(Q₂)O—;
T₁ is —CR₅R₅—, —N(Q)-, —O—, or —S—;
T₂ is —CR₅R₅—, —N(Q)-, —O—, or —S—;
L₂ is CR₅ or N;

R₃ has the formula:

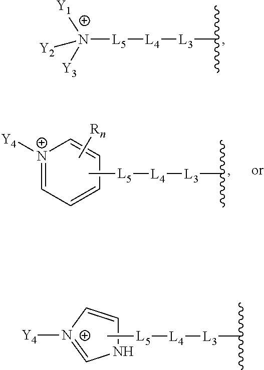

wherein

each of Y₁, Y₂, Y₃, and Y₄, independently, is alkyl, cycloalkyl, aryl, aralkyl, or alkynyl; or

any two of Y₁, Y₂, and Y₃ are taken together with the N atom to which they are attached to form a 3- to 8-member heterocycle; or

Y₁, Y₂, and Y₃ are all be taken together with the N atom to which they are attached to form a bicyclic 5- to 12-member heterocycle;

each Rₙ, independently, is H, halo, cyano, hydroxy, amino, alkyl, alkoxy, cycloalkyl, aryl, heteroaryl, or heterocyclyl;

L₃ is a bond, —N(Q)-, —O—, —S—, —(CR₅R₆)ₐ—, —C(O)—, or a combination of any two of these;

L₄ is a bond, —N(Q)-, —O—, —S—, —(CR₅R₆)ₐ—, —C(O)—, or a combination of any two of these;

L₅ is a bond, —N(Q)-, —O—, —S—, —(CR₅R₆)ₐ—, —C(O)—, or a combination of any two of these;

each occurrence of R₅ and R₆ is, independently, H, halo, cyano, hydroxy, amino, alkyl, alkoxy, cycloalkyl, aryl, heteroaryl, or heterocyclyl; or two R₅ groups on adjacent carbon atoms are taken together to form a double bond between their respective carbon atoms; or two R₅ groups on adjacent carbon atoms and two R₆ groups on the same adjacent carbon atoms are taken together to form a triple bond between their respective carbon atoms;

each a, independently, is 0, 1, 2, or 3;

wherein

an R₅ or R₆ substituent from any of L₃, L₄, or L₅ is optionally taken with an R₅ or R₆ substituent from any of L₃, L₄, or L₅ to form a 3- to 8-member cycloalkyl, heterocyclyl, aryl, or heteroaryl group; and

any one of Y₁, Y₂, or Y₃, is optionally taken together with an R₅ or R₆ group from any of L₃, L₄, and L₅, and atoms to which they are attached, to form a 3- to 8-member heterocyclyl group;

each Q, independently, is H, alkyl, acyl, cycloalkyl, alkenyl, alkynyl, aryl, heteroaryl or heterocyclyl; and

each Q₂, independently, is O, S, N(O)(O), alkyl or alkoxy.

Exemplary lipid-siRNA formulations are as follows:

| | | cationic lipid/non-cationic | |
|---|---|---|---|
| | | lipid/cholesterol/PEG-lipid | |
| | | conjugate | |
| | Cationic Lipid | Lipid:siRNA ratio | Process |
| SNALP | 1,2-Dilinolenyloxy-N,N- | DLinDMA/DPPC/Cholesterol/PEG-cDMA | |
| | dimethylaminopropane (DLinDMA) | (57.1/7.1/34.4/1.4) | |
| | | lipid:siRNA~7:1 | |
| SNALP-XTC | 2,2-Dilinoleyl-4-dimethylaminoethyl-[1,3]- | XTC/DPPC/Cholesterol/PEG-cDMA | |
| | dioxolane (XTC) | 57.1/7.1/34.4/1.4 | |
| | | lipid:siRNA~7:1 | |
| LNP05 | 2,2-Dilinoleyl-4-dimethylaminoethyl-[1,3]- | XTC/DSPC/Cholesterol/PEG-DMG | Extrusion |
| | dioxolane (XTC) | 57.5/7.5/31.5/3.5 | |
| | | lipid:siRNA~6:1 | |
| LNP06 | 2,2-Dilinoleyl-4-dimethylaminoethyl-[1,3]- | XTC/DSPC/Cholesterol/PEG-DMG | Extrusion |
| | dioxolane (XTC) | 57.5/7.5/31.5/3.5 | |
| | | lipid:siRNA~11:1 | |
| LNP07 | 2,2-Dilinoleyl-4-dimethylaminoethyl-[1,3]- | XTC/DSPC/Cholesterol/PEG-DMG | In-line |
| | dioxolane (XTC) | 60/7.5/31/1.5, | mixing |
| | | lipid:siRNA~6:1 | |
| LNP08 | 2,2-Dilinoleyl-4-dimethylaminoethyl-[1,3]- | XTC/DSPC/Cholesterol/PEG-DMG | In-line |
| | dioxolane (XTC) | 60/7.5/31/1.5, | mixing |
| | | lipid:siRNA~11:1 | |
| LNP09 | 2,2-Dilinoleyl-4-dimethylaminoethyl-[1,3]- | XTC/DSPC/Cholesterol/PEG-DMG | In-line |
| | dioxolane (XTC) | 50/10/38.5/1.5 | mixing |
| | | Lipid:siRNA 10:1 | |
| LNP10 | (3aR,5s,6aS)-N,N-dimethyl-2,2-di((9Z,12Z)- | ALN100/DSPC/Cholesterol/PEG-DMG | In-line |
| | octadeca-9,12-dienyl)tetrahydro-3aH- | 50/10/38.5/1.5 | mixing |
| | cyclopenta[d][1,3]dioxol-5-amine (ALN100) | Lipid:siRNA 10:1 | |
| LNP11 | (6Z,9Z,28Z,31Z)-heptatriaconta-6,9,28,31- | MC-3/DSPC/Cholesterol/PEG-DMG | In-line |
| | tetraen-19-yl 4-(dimethylamino)butanoate | 50/10/38.5/1.5 | mixing |
| | (MC3) | Lipid:siRNA 10:1 | |
| LNP12 | 1,1′-(2-(4-(2-((2-(bis(2- | Tech G1/DSPC/Cholesterol/PEG-DMG | In-line |
| | hydroxydodecyl)amino)ethyl)(2- | 50/10/38.5/1.5 | mixing |
| | hydroxydodecyl)amino)ethyl)piperazin-1- | Lipid:siRNA 10:1 | |
| | yl)ethylazanediyl)didodecan-2-ol (Tech G1) | | |

LNP09 formulations and XTC comprising formulations are described, e.g., in U.S. Provisional Ser. No. 61/239,686, filed Sep. 3, 2009; LNP11 formulations and MC3 comprising formulations are described, e.g., in U.S. Provisional Ser. No. 61/244,834, filed Sep. 22, 2009; LNP12 formulations and TechG1 comprising formulations are described, e.g., in U.S. Provisional Ser. No. 61/175,770, filed May 5, 2009.

In one embodiment, the reagent that facilitates RNA effector molecule uptake used herein comprises a charged lipid as described in U.S. Application Ser. No. 61/267,419, filed 7 Dec. 2009, and U.S. Application Ser. No. 61/334,398, filed 13 May 2010. In various embodiments, the RNA effector molecule composition described herein comprises “Lipid H”, “Lipid K” (e.g., K8), “Lipid L” (e.g., L8), “Lipid M”, “Lipid P” (e.g., P8), or “Lipid R”, whose formulas are indicated as follows:

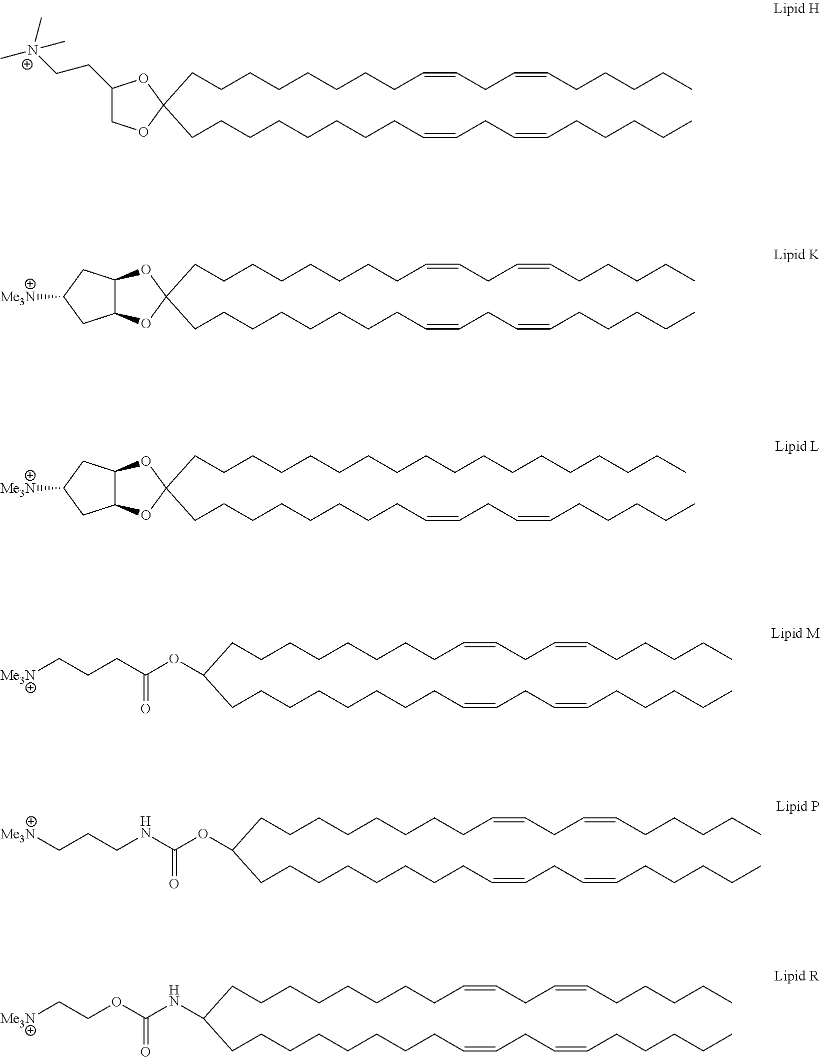

In another embodiment, the RNA effector molecule composition described herein further comprises a lipid formulation comprising a lipid selected from the group consisting of Lipid H, Lipid K, Lipid L, Lipid M, Lipid P, and Lipid R, and further comprises a neutral lipid and a sterol. In particular embodiments, the lipid formulation comprises between about 25 mol % to 100 mol % of the lipid, inclusive. In another embodiment, the lipid formulation comprises between 0 mol % to 50 mol % cholesterol, inclusivel. In still another embodiment, the lipid formulation comprises between 30 mol % to 65 mol % of a neutral lipid, inclusive. In particular embodiments, the lipid formulation comprises the relative mol % of the components as listed in Table 4, as follows:

TABLE 4
Example lipid formulae

| Series | Lipid (Mol %) | DOPE | Chol |
|---|---|---|---|
| 1 | 45.56 | 54.44 | 0 |
| 2 | 48.08 | 51.92 | 0 |
| 3 | 50.60 | 49.40 | 0 |
| 4 | 53.10 | 46.90 | 0 |
| 5 | 52.73 | 37.27 | 10 |
| 6 | 52.92 | 42.08 | 5 |
| 7 | 53.01 | 44.49 | 2.5 |
| 8 | 47.94 | 47.06 | 5 |

Formulations prepared by either the standard or extrusion-free method can be characterized in similar manners. For example, formulations are typically characterized by visual inspection. They should be whitish translucent solutions free from aggregates or sediment. Particle size and particle size distribution of lipid-nanoparticles can be measured by light scattering using, for example, a Malvern Zetasizer Nano ZS (Malvern, USA). Particles should be about 20-300 nm, such as 40-100 nm in size. The particle size distribution should be unimodal. The total sRNA effector molecule concentration in the formulation, as well as the entrapped fraction, is estimated using a dye exclusion assay. A sample of the formulated RNA effector molecule can be incubated with an RNA-binding dye, such as Ribogreen (Molecular Probes) in the presence or absence of a formulation disrupting surfactant, e.g., 0.5% Triton-X100. The total RNA effector molecule in the formulation can be determined by the signal from the sample containing the surfactant, relative to a standard curve. The entrapped fraction is determined by subtracting the “free” RNA effector molecule content (as measured by the signal in the absence of surfactant) from the total RNA effector molecule content. Percent entrapped RNA effector molecule is typically >85%. For lipid nanoparticle formulation, the particle size is at least 30 nm, at least 40 nm, at least 50 nm, at least 60 nm, at least 70 nm, at least 80 nm, at least 90 nm, at least 100 nm, at least 110 nm, or at least 120 nm. The suitable range is typically about at least 50 nm to about at least 110 nm, about at least 60 nm to about at least 100 nm, or about at least 80 nm to about at least 90 nm, inclusive.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are taken up by macrophages in vivo. In order to cross intact cell membranes, lipid vesicles must pass through a series of fine pores, each with a diameter less than 50 nm, under the influence of a suitable transdermal gradient. Therefore, it is desirable to use a liposome which is highly deformable and able to pass through such fine pores.

Further advantages of liposomes include: liposomes obtained from natural phospholipids are biocompatible and biodegradable; liposomes can incorporate a wide range of water and lipid soluble drugs; and liposomes can protect encapsulated drugs in their internal compartments from metabolism and degradation. See, e.g., Wang et al., DRUG DELIV. PRINCIPLES & APPL. (John Wiley & Sons, Hoboken, N.J., 2005); Rosoff, 1988. Important considerations in the preparation of liposome formulations are the lipid surface charge, vesicle size and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes and as the merging of the liposome and cell progresses, the liposomal contents are emptied into the cell where the active agent can act. Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Liposomes fall into two broad classes. Cationic liposomes are positively charged liposomes which interact with the negatively charged polynucleotide molecules to form a stable complex. The positively charged polynucleotide/liposome complex binds to the negatively charged cell surface and is internalized in an endosome. Due to the acidic pH within the endosome, the liposomes are ruptured, releasing their contents into the cell cytoplasm. Wang et al., 147 Biochem. Biophys. Res. Commun, 980-85 (1987).

Liposomes which are pH-sensitive or negatively-charged, entrap polynucleotide rather than complex with it. Because both the polynucleotide and the lipid are similarly charged, repulsion rather than complex formation occurs. Nevertheless, some polynucleotide is entrapped within the aqueous interior of these liposomes. pH-sensitive liposomes have been used to deliver DNA encoding the thymidine kinase gene to cell monolayers in culture. Expression of the exogenous gene was detected in the target cells. Zhou et al., 19 J. Controlled Release 269-74 (1992).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES). Allen et al., 223 FEBS Lett. 42 (1987); Wu et al., 53 Cancer Res. 3765 (1993).

Various liposomes comprising one or more glycolipids are known in the art. Papahadjopoulos et al. (507 Ann. N.Y. Acad. Sci. 64 (1987)), reported the ability of monosialoganglioside GM1, galactocerebroside sulfate and phosphatidylinositol to improve blood half-lives of liposomes. These findings were expounded upon by Gabizon et al. (85 PNAS 6949 (1988)). U.S. Pat. No. 4,837,028 and WO 88/04924, both to Allen et al., disclose liposomes comprising (1) sphingomyelin and (2) the ganglioside GM1 or a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (53 Bull. Chem. Soc. Jpn. 2778 (1980)) described liposomes comprising a nonionic detergent, 2C1215G, that contains a PEG moiety. Ilium et al. (167 FEBS Lett. 79 (1984)), noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. No. 4,426,330 and No. 4,534,899). In addition, antibodies can be conjugated to a polyakylene derivatized liposome (see e.g., PCT Application US 2008/0014255). Klibanov et al. (268 FEBS Lett. 235 (1990)), described experiments demonstrating that liposomes comprising phosphatidylethanolamine (PE) derivatized with PEG or PEG stearate have significant increases in blood circulation half-lives. Blume et al. (1029 Biochim. Biophys. Acta 1029, (1990)), extended such observations to other PEG-derivatized phospholipids, e.g., DSPE-PEG, formed from the combination of distearoylphosphatidylethanolamine (DSPE) and PEG. Liposomes having covalently bound PEG moieties on their external surface are described in European Patent No. 0 445 131 B1 and WO 90/04384 to Fisher.

Liposome compositions containing 1-20 mole percent of PE derivatized with PEG, and methods of use thereof, are described by Woodle et al. (U.S. Pat. No. 5,013,556; No. 5,356,633) and Martin et al. (U.S. Pat. No. 5,213,804; European Patent No. 0 496813 B1). Liposomes comprising a number of other lipid-polymer conjugates are disclosed in WO 91/05545 and U.S. Pat. No. 5,225,212 and in WO 94/20073. Liposomes comprising PEG-modified ceramide lipids are described in WO 96/10391. U.S. Pat. No. 5,540,935 and No. 5,556,948 describe PEG-containing liposomes that can be further derivatized with functional moieties on their surfaces. Methods and compositions relating to liposomes comprising PEG can be found in, e.g., U.S. Pat. No. 6,049,094; No. 6,224,903; No. 6,270,806; No. 6,471,326; No. 6,958,241.

As noted above, liposomes can optionally be prepared to contain surface groups, such as antibodies or antibody fragments, small effector molecules for interacting with cell-surface receptors, antigens, and other like compounds, and these groups can facilitate delivery of liposomes and their contents to specific cell populations. Such ligands can be included in the liposomes by including in the liposomal lipids a lipid derivatized with the targeting molecule, or a lipid having a polar-head chemical group that can be derivatized with the targeting molecule in preformed liposomes. Alternatively, a targeting moiety can be inserted into preformed liposomes by incubating the preformed liposomes with a ligand-polymer-lipid conjugate.

Lipids can be derivatized using a variety of targeting moieties, such as ligands, cell surface receptors, glycoproteins, vitamins (e.g., riboflavin) and monoclonal antibodies by covalently attaching the ligand to the free distal end of a hydrophilic polymer chain, which is attached at its proximal end to a vesicle-forming lipid. There are a wide variety of techniques for attaching a selected hydrophilic polymer to a selected lipid and activating the free, unattached end of the polymer for reaction with a selected ligand, and as noted above, the hydrophilic polymer polyethyleneglycol (PEG) has been studied widely. Allen et al., 1237 Biochem. Biophys. Acta 99-108 (1995); Zalipsky, 4 Bioconj. Chem. 296-99 (1993); Zalipsky et al., 353 FEBS Lett. 1-74 (1994); Zalipsky et al., Bioconj. Chem. 705-08 (1995); Zalipsky, in STEALTH LIPOSOMES (Lasic & Martin, eds. CRC Press, Boca Raton, Fla., 1995).

A number of liposomes comprising nucleic acids are known in the art, such as methods for encapsulating high molecular weight nucleic acids in liposomes. WO 96/40062. U.S. Pat. No. 5,264,221 to Tagawa et al. discloses protein-bonded liposomes and asserts that the contents of such liposomes can include a dsRNA. U.S. Pat. No. 5,665,710 to Rahman et al. describes certain methods of encapsulating oligodeoxynucleotides in liposomes. WO 97/04787 to Love et al. discloses liposomes comprising dsRNAs targeted to the raf gene. In addition, methods for preparing a liposome composition comprising a nucleic acid can be found in, e.g., U.S. Pat. No. 6,011,020; No. 6,074,667; No. 6,110,490; No. 6,147,204; No. 6,271,206; No. 6,312,956; No. 6,465,188; No. 6,506,564; No. 6,750,016; No. 7,112,337.

Transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes are adaptable to the environment in which they are used, e.g., they are self-optimizing, self-repairing, frequently reach their targets without fragmenting, and often self-loading. To make transfersomes it is possible to add surface edge-activators, usually surfactants, to a standard liposomal composition.

Emulsions

The compositions of the present invention can be prepared and formulated as emulsions. Emulsions are typically heterogenous systems of one liquid dispersed in another in the form of droplets usually exceeding 0.1 nm in diameter. See, e.g., Ansel's PHARM. DOSAGE FORMS & DRUG DELIV. SYS. (8th ed. Allen et al., eds., Lippincott Williams & Wilkins, NY, 2004); Idson, in 1 PHARM. DOSAGE FORMS 199 (Lieberman et al., eds., Marcel Dekker, Inc., NY, 1988); Rosoff, in 1 PHARM. DOSAGE FORMS 245 (Lieberman et al., eds., Marcel Dekker, Inc., NY, 1988); Block in 2 PHARM. DOSAGE FORMS 335 (Lieberman et al., eds., Marcel Dekker, Inc., NY, 1988); Higuchi et al., in REMINGTON'S PHARM. SCI. 301 (Mack Publishing Co., Easton, Pa., 1985). Emulsions are often biphasic systems comprising two immiscible liquid phases intimately mixed and dispersed with each other.

In general, emulsions can be of either the water-in-oil (w/o) or the oil-in-water (o/w) variety. When an aqueous phase is finely divided into and dispersed as minute droplets into a bulk oily phase, the resulting composition is called a water-in-oil (w/o) emulsion. Alternatively, when an oily phase is finely divided into and dispersed as minute droplets into a bulk aqueous phase, the resulting composition is called an oil-in-water (o/w) emulsion. Emulsions can contain additional components in addition to the dispersed phases, and the active drug which can be present as a solution in either the aqueous phase, oily phase or itself as a separate phase. Pharmaceutical excipients such as emulsifiers, stabilizers, dyes, and anti-oxidants can also be present in emulsions as needed. Pharmaceutical emulsions can also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous phase provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion can be a semisolid or a solid, as is the case of emulsion-style ointment bases and creams. Other means of stabilizing emulsions entail the use of emulsifiers that can be incorporated into either phase of the emulsion. Emulsifiers can broadly be classified into four categories: synthetic surfactants, naturally occurring emulsifiers, absorption bases, and finely dispersed solids. See, e.g., ANSEL'S PHARM. DOSAGE FORMS & DRUG DELIV. SYS., 2004; Idson, in PHARM. DOSAGE FORMS, 1988.

Synthetic surfactants, also known as surface active agents, have found wide applicability in the formulation of emulsions and have been reviewed in the literature. See, e.g., ANSEL'S PHARM. DOSAGE FORMS & DRUG DELIV. SYS., 2004; Idson, in PHARM. DOSAGE FORMS, 1988; Rieger, in PHARM. DOSAGE FORMS, 1988. Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants can be classified into different classes based on the nature of the hydrophilic group: nonionic, anionic, cationic and amphoteric. See, e.g., ANSEL′S PHARM. DOSAGE FORMS & DRUG DELIV. SYS., 2004; Idson, in PHARM. DOSAGE FORMS, 1988; Rieger, in PHARM. DOSAGE FORMS, 1988.

Naturally occurring emulsifiers used in emulsion formulations include lanolin, beeswax, phosphatides, lecithin and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives and antioxidants. Block, in 1 PHARM. DOSAGE FORMS 335 (Lieberman et al., eds., Marcel Dekker, Inc., NY, 1988); Idson, in PHARM. DOSAGE FORMS (1988).

Hydrophilic colloids or hydrocolloids include naturally occurring gums and synthetic polymers such as polysaccharides (e.g., acacia, agar, alginic acid, carrageenan, guar gum, karaya gum, and tragacanth), cellulose derivatives (e.g., carboxymethylcellulose and carboxypropylcellulose), and synthetic polymers (e.g., carbomers, cellulose ethers, and carboxyvinyl polymers). These disperse or swell in water to form colloidal solutions that stabilize emulsions by forming strong interfacial films around the dispersed-phase droplets and by increasing the viscosity of the external phase.

Because emulsions often contain a number of ingredients such as carbohydrates, proteins, sterols and phosphatides that can readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used can be free radical scavengers such as tocopherols, alkyl gallates, butylated hydroxyanisole, butylated hydroxytoluene, or reducing agents such as ascorbic acid and sodium metabisulfite, and antioxidant synergists such as citric acid, tartaric acid, and lecithin.

In one embodiment, the compositions of RNA effector molecules and nucleic acids are formulated as microemulsions. A microemulsion can be defined as a system of water, oil and amphiphile which is a single optically isotropic and thermodynamically stable liquid solution. See, e.g., ANSEL'S PHARM. DOSAGE FORMS & DRUG DELIV. SYS. (8th ed., Allen et al, eds., Lippincott Williams & Wilkins, NY, 2004); Rosoff, in PHARM. DOSAGE FORMS, 1988.

Typically, microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules. Leung & Shah, in CONTROLLED RELEASE DRUGS: POLYMERS & AGGREGATE SYS. 185-215 (Rosoff, ed., VCH Publishers, NY, 1989). Microemulsions commonly are prepared via a combination of three to five components that include oil, water, surfactant, cosurfactant and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules. Schott, in REMINGTON'S PHARM. SCI. 271 (1985).

The phenomenological approach utilizing phase diagrams has been extensively studied and has yielded a comprehensive knowledge, to one skilled in the art, of how to formulate microemulsions. See, e.g., ANSEL'S PHARM. DOSAGE FORMS & DRUG DELIV. SYS. (8th ed., Allen et al, eds., Lippincott Williams & Wilkins, NY, 2004); Rosoff, 1988; Block, 1988. Compared to conventional emulsions, microemulsions offer the advantage of solubilizing water-insoluble drugs in a formulation of thermodynamically stable droplets that are formed spontaneously.

Microemulsions can include surfactants, discussed further herein, not limited to ionic surfactants, non-ionic surfactants, Brij 96, polyoxyethylene oleyl ethers, polyglycerol fatty acid esters, tetraglycerol monolaurate (ML310), tetraglycerol monooleate (M0310), hexaglycerol monooleate (PO310), hexaglycerol pentaoleate (PO500), decaglycerol monocaprate (MCA750), decaglycerol monooleate (M0750), decaglycerol sequioleate (S0750), decaglycerol decaoleate (DA0750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions can, however, be prepared without the use of cosurfactants and alcohol-free self-emulsifying microemulsion systems are known in the art. The aqueous phase can typically be, but is not limited to, water, an aqueous solution of the drug, glycerol, PEG300, PEG400, polyglycerols, propylene glycols, and derivatives of ethylene glycol. The oil phase can include, but is not limited to, materials such as Captex 300, Captex 355, Capmul MCM, fatty acid esters, medium chain (C8-C12) mono, di, and tri-glycerides, polyoxyethylated glyceryl fatty acid esters, fatty alcohols, polyglycolized glycerides, saturated polyglycolized C8-C10 glycerides, vegetable oils and silicone oil.

Microemulsions afford advantages of better drug solubilization, protection of drug from enzymatic hydrolysis, possible enhancement of drug absorption due to surfactant-induced alterations in membrane fluidity and permeability, ease of preparation, and decreased toxicity. See, e.g., U.S. Pat. No. 6,191,105; No. 7,063,860; No. 7,070,802; No. 7,157,099; Constantinides et al., 11 Pharm. Res. 1385 (1994); Ho et al., 85 J. Pharm. Sci. 138-43 (1996). Often, microemulsions can form spontaneously when their components are brought together at ambient temperature. This can be particularly advantageous when formulating thermolabile drugs, peptides or RNA effector molecules.

Microemulsions of the present invention can also contain additional components and additives such as sorbitan monostearate (Grill 3), Labrasol, and penetration enhancers to improve the properties of the formulation and to enhance the absorption of the RNA effector molecules and nucleic acids of the present invention. Penetration enhancers used in the microemulsions of the present invention can be classified as belonging to one of five broad categories—surfactants, fatty acids, bile salts, chelating agents, and non-chelating non-surfactants. Lee et al., Crit. Rev. Therapeutic Drug Carrier Sys. 92 (1991).

There are many organized surfactant structures besides microemulsions that have been studied and used for the formulation of drugs. These include monolayers, micelles, bilayers and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Surfactants

In some embodiments, RNA effector molecules featured in the invention are formulated in conjunction with one or more penetration enhancers, surfactants and/or chelators. Suitable surfactants include fatty acids and/or esters or salts thereof, bile acids and/or salts thereof. Suitable bile acids/salts include chenodeoxycholic acid (CDCA) and ursodeoxychenodeoxy-cholic acid (UDCA), cholic acid, dehydrocholic acid, deoxycholic acid, glucholic acid, glycholic acid, glycodeoxycholic acid, taurocholic acid, taurodeoxycholic acid, sodium tauro-24,25-dihydro-fusidate and sodium glycodihydrofusidate. Suitable fatty acids include arachidonic acid, undecanoic acid, oleic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a monoglyceride, a diglyceride or a pharmaceutically acceptable salt thereof (e.g., sodium). In some embodiments, combinations of penetration enhancers are used, for example, fatty acids/salts in combination with bile acids/salts. One exemplary combination is the sodium salt of lauric acid, capric acid and UDCA. Further penetration enhancers include polyoxyethylene-9-lauryl ether, polyoxyethylene-20-cetyl ether.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations. See e.g., Malmsten, SURFACTANTS & POLYMERS IN DRUG DELIV. (Informa Health Care, NY, 2002); Rieger, in PHARM. DOSAGE FORMS 285 (Marcel Dekker, Inc., NY, 1988).

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines The quaternary ammonium salts are the most used members of this class. If the surfactant molecule has the ability to carry either a positive or negative charge, the surfactant is classified as amphoteric. Amphoteric surfactants include acrylic acid derivatives, substituted alkylamides, N-alkylbetaines and phosphatides.

Penetration Enhancers

In one embodiment, the present invention employs various penetration enhancers to effect the efficient delivery of nucleic acids, particularly RNA effector molecules, to the cell. Most drugs are present in solution in both ionized and nonionized forms. Usually, only lipid soluble or lipophilic drugs readily cross cell membranes. It has been discovered that even non-lipophilic drugs can cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers can be classified as belonging to one of five broad categories: surfactants, fatty acids, bile salts, chelating agents, and non-chelating non-surfactants. See, e.g., Malmsten, 2002; Lee et al., Crit. Rev. Therapeutic Drug Carrier Sys. 92 (1991).

In connection with the present invention, penetration enhancers include surfactants (or “surface-active agents”), which are chemical entities that, when dissolved in an aqueous solution, reduce the surface tension of the solution or the interfacial tension between the aqueous solution and another liquid, with the result that absorption of RNA effector molecules through cellular membranes and other biological barriers is enhanced. In addition to bile salts and fatty acids, these penetration enhancers include, for example, sodium lauryl sulfate, polyoxyethylene-9-lauryl ether and polyoxyethylene-20-cetyl ether) (see, e.g., Malmsten, 2002; Lee et al., 1991); and perfluorochemical emulsions, such as FC-43 (Takahashi et al., 40 J. Pharm. Pharmacol. 252 (1988)).

Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-rac-glycerol), dilaurin, caprylic acid, arachidonic acid, glycerol 1-monocaprate, 1-dodecylazacyclo-heptan-2-one, acylcarnitines, acylcholines, C1-20 alkyl esters thereof (e.g., methyl, isopropyl and t-butyl), and mono- and di-glycerides thereof (i.e., oleate, laurate, caprate, myristate, palmitate, stearate, linoleate, etc.). See, e.g., Touitou et al., ENHANCEMENT IN DRUG DELIV. (CRC Press, Danvers, Mass., 2006); Lee et al., 1991; Muranishi, 7 Crit. Rev. Therapeutic Drug Carrier Sys. 1-33 (1990); El Hariri et al., 44 J. Pharm. Pharmacol. 651-54 (1992).

The physiological role of bile includes the facilitation of dispersion and absorption of lipids and fat-soluble vitamins. See, e.g., Malmsten, 2002; Brunton, Chapt. 38 in GOODMAN & GILMAN'S PHARMACOLOGICAL BASIS THERAPEUTICS, 9TH ED. 934-35 (Hardman et al., eds., McGraw-Hill, NY, 1996). Various natural bile salts, and their synthetic derivatives, act as penetration enhancers. Thus the term “bile salts” includes any of the naturally occurring components of bile as well as any of their synthetic derivatives. Suitable bile salts include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate and polyoxyethylene-9-lauryl ether (POE) (see e.g., Malmsten, 2002; Lee et al., 1991; Swinyard, Chapt. 39 in REMINGTON'S PHARM. SCI., 18th Ed. 782-83 (Gennaro, ed., Mack Publishing Co., Easton, Pa., 1990); Muranishi, 1990; Yamamoto et al., 263 J. Pharm. Exp. Ther. 25 (1992); Yamashita et al., 79 J. Pharm. Sci. 579-83 (1990).

Chelating agents, as used in connection with the present invention, can be defined as compounds that remove metallic ions from solution by forming complexes therewith, with the result that absorption of RNA effector molecules through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents. Jarrett, 618 J. Chromatogr. 315-39 (1993). Suitable chelating agents include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines). See, e.g., Katdare et al., EXCIPIENT DEVEL. PHARM. BIOTECH. & DRUG DELIV. (CRC Press, Danvers, Mass., 2006); Lee et al., 1991; Muranishi, 1990; Buur et al., 14 J. Control Rel. 43-51 (1990).

As used herein, non-chelating non-surfactant penetration enhancing compounds can be defined as compounds that demonstrate insignificant activity as chelating agents or as surfactants but that nonetheless enhance absorption of RNA effector molecules through the alimentary mucosa. See e.g., Muranishi, 1990. This class of penetration enhancers include, for example, unsaturated cyclic ureas, 1-alkyl- and 1-alkenylazacyclo-alkanone derivatives (Lee et al., 1991); and non-steroidal anti-inflammatory agents such as diclofenac sodium, indomethacin and phenylbutazone (Yamashita et al., 1987).

Agents that enhance uptake of RNA effector molecules at the cellular level can also be added to the pharmaceutical and other compositions of the present invention. For example, cationic lipids, such as lipofectin (U.S. Pat. No. 5,705,188), cationic glycerol derivatives, and polycationic molecules, such as polylysine (WO 97/30731), are also known to enhance the cellular uptake of dsRNAs. Examples of commercially available transfection reagents include, for example LIPOFECTAMINE™, LIPOFECTAMINE 2000™, 293FECTIN™, CELLFECTIN™, DMRIE-C™, FREESTYLE™ MAX, LIPOFECTAMINE™ 2000 CD, LIPOFECTAMINE™, RNAiMAX, OLIGOFECTAMINE™, and OPTIFECT™ (each of the foregoing Invitrogen, Carlsbad, Calif.) transfection reagents; and X-tremeGENE Q2 Transfection Reagent (Roche Applied Science; Grenzacherstrasse, Switzerland), DOTAP Liposomal Transfection Reagent (Avante Polar Lipids, Inc., Alabaster, AL), DOSPER Liposomal Transfection Reagent (Roche), or FuGENE®, TRANSFECTAM® Reagent, TRANSFAST™ Transfection Reagent, TFX™-20 Reagent, or TFX™-50 Reagent (each of the foregoing Promega, Madison, Wis.); DREAMFECT™ (OZ Biosciences, Marseille, France), EcoTransfect (OZ Biosciences); TRANSPASS® D1 Transfection Reagent (New England Biolabs; Ipswich, Mass.); LYOVEC™/LIPOGEN™ (InvivoGen; San Diego, Calif.); PerFectin, NEUROPORTER, GENEPORTER, GENEPORTER 2, CYTOFECTIN, BACULOPORTER, or TROGANPORTERT™ transfection reagents (each of the foregoing Genlantis San Diego, Calif.); RIBOFECT (Bioline; Taunton, Mass., U.S.), PLASFECT (Bioline); UNIFECTOR, SUREFECTOR, or HIFECT™ (each from B-Bridge International, Mountain View, Calif.), among others.

Additional Carriers

Other agents can be utilized to enhance the penetration of the administered nucleic acids, including glycols such as ethylene glycol and propylene glycol, pyrrols such as 2-pyrrol, azones, and terpenes such as limonene and menthone.

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal.

The compositions of the present invention can additionally contain other adjunct components so long as such materials, when added, do not unduly interfere with the biological activities of the components of the compositions of the present invention. The formulations can be sterilized and, if desired, mixed with auxiliary agents that do not deleteriously interact with the RNA effector molecules of the formulation.

Aqueous suspensions can contain substances which increase the viscosity of the suspension including, for example, sodium carboxymethylcellulose, sorbitol and/or dextran. The suspension can also contain stabilizers.

Toxicity and therapeutic efficacy of such compounds can be determined by standard pharmaceutical procedures in eggs or in cells, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are particularly useful. The data obtained from in vitro and in vivo studies can be used in formulating a range of dosages for use in the instant methods. The dosage of compositions featured in the invention lies generally within a range of concentrations that includes the ED₅₀ with little or no toxicity. The dosage can vary within this range depending upon the dosage form employed and the route of administration utilized.

In yet another aspect, the invention provides a method for inhibiting the expression of a target gene in a cell of an embryonated egg by administering a composition featured in the invention to the egg cell such that expression of the target gene is decreased for an extended duration, e.g., at least 2 days, 3 days, 4 days, 5 days, 6 days, or more, e.g., one week, or longer. The effect of the decreased expression of the target gene preferably results in a decrease in levels of the target protein or pathway impacted by the target gene by at least 10%, at least 15%, at least 20%, at least 25%, at least 30%, at least 40%, at least 50%, or at least 60%, or more, as compared to pretreatment levels.

### VI. KITS AND ASSAYS

In some embodiments, kits are provided for testing the effect of a RNA effector molecule or a series of RNA effector molecules on the production of a biological product by the egg, where the kits comprise a substrate having one or more assay surfaces suitable for culturing harvested cells under conditions that allow production of a biological product. In some embodiments, the exterior of the substrate comprises wells, indentations, demarcations, or the like at positions corresponding to the assay surfaces. In some embodiments, the wells, indentations, demarcations, or the like retain fluid, such as cell culture media, over the assay surfaces.

In some embodiments, the assay surfaces on the substrate are sterile and are suitable for culturing cells under conditions representative of the culture conditions in the egg for production of the biological product. Advantageously, kits provided herein offer a rapid, cost-effective means for testing a wide-range of agents and/or conditions on the production of a biological product, allowing the manipulation of the egg cell to be established prior to full-scale production of the biological product in eggs.

In some embodiments, one or more assay surfaces of the substrate comprise a concentrated test agent, such as a RNA effector molecule, such that the addition of suitable media to the assay surfaces results in a desired concentration of the RNA effector molecule surrounding the assay surface. In some embodiments, the RNA effector molecules can be printed or ingrained onto the assay surface, or provided in a lyophilized form, e.g., within wells, such that the effector molecules can be reconstituted upon addition of an appropriate amount of media. In some embodiments, the RNA effector molecules are reconstituted by plating harvested embryontaed egg cells onto assay surfaces of the substrate.

In some embodiments, kits provided herein further comprise cell culture media suitable for culturing an egg cell under conditions allowing for the production of a biological product of interest. The media can be in a ready to use form or can be concentrated (e.g., as a stock solution), lyophilized, or provided in another reconstitutable form.

In further embodiments, kits provided herein further comprise one or more reagents suitable for detecting production of the biological product by the egg. In further embodiments, the reagent(s) are suitable for detecting a property of the egg, such as maximum cell density, embryo viability, or the like, which is indicative of production of the desired biological product. In some embodiments, the reagent(s) are suitable for detecting the biological product or a property thereof, such as the in vitro or in vivo biological activity, homogeneity, or structure of the biological product, such as infectivity harvested virus (e.g., pfu/egg).

In some embodiments, one or more assay surfaces of the substrate further comprise a carrier for which facilitates uptake of RNA effector molecules by egg cells. Carriers for RNA effector molecules are known in the art and are described herein. For example, in some embodiments, the carrier is a lipid formulation such as LIPOFECTAMINE™ transfection reagent (Invitrogen) or a related formulation. Examples of such carrier formulations are described herein. In some embodiments, the reagent that facilitates RNA effector molecule uptake comprises a charged lipid, an emulsion, a liposome, a cationic or non-cationic lipid, an anionic lipid, a transfection reagent or a penetration enhancer as described throughout the application herein. In particular embodiments, the reagent that facilitates RNA effector molecule uptake comprises a charged lipid as described in U.S. Application Ser. No. 61/267,419, filed on Dec. 7, 2009, and U.S. Application Ser. No. 61/334,398, filed Can 13, 2010.

In some embodiments, one or more assay surfaces of the substrate comprise a RNA effector molecule or series of RNA effector molecules and a carrier, each in concentrated form, such that plating test cells onto the assay surface(s) results in a concentration the RNA effector molecule(s) and the carrier effective for facilitating uptake of the RNA effector molecule(s) by the cells and modulation of the expression of one or more genes targeted by the RNA effector molecules.

In some embodiments, the substrate further comprises a matrix which facilitates 3-dimensional cell growth and/or production of the biological product by the cells. In further embodiments, the matrix facilitates anchorage-dependent growth of cells. Non-limiting examples of matrix materials suitable for use with various kits described herein include agar, agarose, methylcellulose, alginate hydrogel (e.g., 5% alginate +5% collagen type I), chitosan, hydroactive hydrocolloid polymer gels, polyvinyl alcohol-hydrogel (PVA-H), polylactide-co-glycolide (PLGA), collagen vitrigel, PHEMA (poly(2-hydroxylmethacrylate)) hydrogels, PVP/PEO hydrogels, BD PURAMATRIX™ hydrogels, and copolymers of 2-methacryloyloxyethyl phosphorylcholine (MPC).

In some embodiments, the substrate comprises a microarray plate, a biochip, or the like which allows for the high-throughput, automated testing of a range of test agents, conditions, and/or combinations thereof on the production of a biological product by egg cells. For example, the substrate can comprise a 2-dimensional microarray plate or biochip having m columns and n rows of assay surfaces (e.g., residing within wells) which allow for the testing of m×n combinations of test agents and/or conditions (e.g., on a 24-, 96- or 384-well microarray plate). The microarray substrates are preferably designed such that all necessary positive and negative controls can be carried out in parallel with testing of the agents and/or conditions.

In further embodiments, kits are provided comprising one or more microarray substrates seeded with a set of RNA effector molecules designed to modulate a particular pathway, function, or property of a cell which affects the production of the biological product. For example, in some embodiments, the RNA effector molecules are directed against target genes comprising a pathway involved in the expression, folding, secretion, post-translational modification, or the viral secretion by the egg cell.

In further embodiments, kits are provided herein comprising one or more microarray substrates seeded with a set of RNA effector molecules designed to address a particular problem or class of problems associated with the production of an immunogenic agent in cell-based systems. For example, in some embodiments, the RNA effector molecules are directed against target genes expressed by latent or endogenous viruses; or involved in cell processes, such as cell cycle progression, cell metabolism or apoptosis which inhibit or interfere production or purification of the biological product. In further embodiments, the RNA effector molecules are directed against target genes that mediate enzymatic degradation, aggregation, misfolding, or other processes that reduce the activity, homogeneity, stability, and/or other qualities of the biological product. In yet further embodiments, the effector molecules are directed against target genes that affect the infectivity of exogenous or adventitious contaminating microbes. In one embodiment, the biological product includes a glycoprotein, and the RNA effector molecules are directed against target genes involved in glycosylation (e.g., fucosylation) and/or proteolytic processing of glycoproteins by the host cell. In another embodiment, the biological product is a multi-subunit recombinant protein and the RNA effector molecules are directed against target genes involved in the folding and/or secretion of the protein by the host cell. In another embodiment, the RNA effector molecules are directed against target genes involved in post-translation modification of the biological product in the cells, such as methionine oxidation, glycosylation, disulfide bond formation, pyroglutamation and/or protein deamidation.

In some embodiments, kits provided herein allow for the selection or optimization of at least one factor for enhancing production of the biological product. For example, the kits can allow for the selection of an RNA effector molecule from among a series of candidate RNA effector molecules, or for the selection of a concentration or concentration range from a wider range of concentrations of a given RNA effector molecule. In some embodiments, the kits allow for selection of one or more RNA effector molecules from a series of candidate RNA effector molecules directed against a common target gene. In further embodiments, the kits allow for selection of one or more RNA effector molecules from a series of candidate RNA effector molecules directed against two or more functionally related target genes or two or more target genes of a common cell pathway.

In some embodiments, kits provided herein allow for the selection or optimization of a combination of two or more factors in the production of a biological product. For example, the kits can allow for the selection of a suitable RNA effector molecule from among a series of candidate RNA effector molecules as well as a concentration of the RNA effector molecule. In further embodiments, kits provided herein allow for the selection of a first RNA effector molecule from a first series of candidate RNA effector molecules and a second RNA effector molecule from a second series of candidate RNA effector molecules. In some embodiments, the first and/or second series of candidate RNA effector molecules are directed against a common target gene. In further embodiments, the first and/or second series of RNA effector molecules are directed against two or more functionally related target genes or two or more target genes of a common host cell pathway.

In another embodiment, a kit for enhancing production of a biological product in a cell, comprising at least a first RNA effector molecule, a portion of which is complementary to at least a first target gene of a latent or endogenous virus; a second RNA effector molecule, a portion of which is complementary to at least a second target gene of the cellular immune response; and, optionally, a third RNA effector molecule, a portion of which is complementary to at least a third target gene of a cellular process. For example, the first target gene is an ERV env gene, the second target gene is an IFNB1, PKR, IRF3 or IFNAR1 gene, and the third target gene is a PTEN, BAK1, BAX or LDHA gene. The kit can further comprise at least additional RNA effector molecule that targets a cellular process including, but not limited to, carbon metabolism and transport, apoptosis, RNAi uptake and/or efficiency, reactive oxygen species production, cell cycle control, protein folding, pyroglutamation protein modification, deamidase, glycosylation, disulfide bond formation, protein secretion, gene amplification, viral replication, viral infection, viral particle release, control of cellular pH, and protein production.

In yet another aspect, the invention provides a method for inhibiting the expression of a target gene in a cell. The method includes administering a composition featured in the invention to the cell such that expression of the target gene is decreased, such as for an extended duration, e.g., at least 2 days, 3 days, 4 days, or more. The RNA effector molecules useful for the methods and compositions featured in the invention specifically target RNAs (primary or processed) of the target gene. Compositions and methods for inhibiting the expression of these target genes using RNA effector molecules can be prepared and performed as described herein.

The invention encompasses vaccine formulations comprising the viral product and a suitable excipient. The virus used in the vaccine formulation can be selected from naturally occurring mutants or variants, mutagenized viruses or genetically engineered viruses. Attenuated strains of segmented RNA viruses can also be generated via reassortment techniques, or by using a combination of the reverse genetics approach and reassortment techniques. Naturally occurring variants include viruses isolated from nature as well as spontaneous occurring variants generated during virus propagation, having an impaired ability to antagonize the cellular IFN response. The attenuated virus can itself be used as the active ingredient in the vaccine formulation. Alternatively, the attenuated virus can be used as the vector or “backbone” of recombinantly produced vaccines. To this end, recombinant techniques such as reverse genetics (or, for segmented viruses, combinations of the reverse genetics and reassortment techniques) can be used to engineer mutations or introduce foreign antigens into the attenuated virus used in the vaccine formulation. In this way, vaccines can be designed for immunization against strain variants, or in the alternative, against completely different infectious agents or disease antigens.

Any practical heterologous gene sequence can be constructed into the viruses of the invention for use in vaccines. Epitopes that induce a protective immune response to any of a variety of pathogens, or antigens that bind neutralizing antibodies can be expressed by or as part of the viruses. For example, heterologous gene sequences that can be constructed into the viruses of the invention for use in vaccines include but are not limited to epitopes of human immunodeficiency virus (HIV) such as gp120; hepatitis B virus surface antigen (HBsAg); the glycoproteins of herpes virus (e.g., gD, gE); VP1 of poliovirus; antigenic determinants of non-viral pathogens such as bacteria and parasites, to name but a few. In another embodiment, all or portions of immunoglobulin genes can be expressed. For example, variable regions of anti-idiotypic immunoglobulins that mimic such epitopes can be constructed into the viruses of the invention. In yet another embodiment, tumor associated antigens can be expressed.

Either a live recombinant viral vaccine or an inactivated recombinant viral vaccine can be formulated. A live vaccine can be preferred because multiplication in the host leads to a prolonged stimulus of similar kind and magnitude to that occurring in natural infections, and therefore, confers substantial, long-lasting immunity. Production of such live recombinant virus vaccine formulations can be accomplished using conventional methods involving propagation of the virus in cell culture or in the allantois of the embryo followed by purification.

Vaccine formulations can include genetically engineered negative strand RNA viruses that have mutations in the NS 1 or analogous gene including but not limited to the truncated NS1 influenza mutants described in the working examples, infra. They can also be formulated using natural variants, such as the A/turkey/Ore/71 natural variant of influenza A, or B/201, and B/AWBY-234, which are natural variants of influenza B. When formulated as a live virus vaccine, a range of about 10⁴ pfu to about 5×10⁶ pfu per dose can be used.

Many methods can be used to introduce the vaccine formulations described herein, these include but are not limited to intranasal, intratracheal, oral, intradermal, intramuscular, intraperitoneal, intravenous, and subcutaneous routes. It can be preferable to introduce the virus vaccine formulation via the natural route of infection of the pathogen for which the vaccine is designed, or via the natural route of infection of the parental attenuated virus. Where a live influenza virus vaccine preparation is used, it can be preferable to introduce the formulation via the natural route of infection for influenza virus. The ability of influenza virus to induce a vigorous secretory and cellular immune response can be used advantageously. For example, infection of the respiratory tract by influenza viruses can induce a strong secretory immune response, for example in the urogenital system, with concomitant protection against a particular disease causing agent.

A vaccine of the present invention could be administered once, or twice or three times with an interval of 2 months to 6 months between doses. Alternatively, a vaccine of the present invention, comprising could be administered as often as needed to an animal or a human being.

The present invention may be as defined in any one of the following numbered paragraphs.

1. A method for producing a biological product in an embryonated egg, comprising:

(a) introducing into the egg at least a first RNA effector molecule, a portion of which is complementary to a target gene;

(b) maintaining the egg for a time sufficient to modulate expression of the at least one of the first and second target genes, and

(c) isolating the biological product from the egg;

wherein the target gene is a gene of a cellular immune response.

2. The method of paragraph 1, wherein the target gene is a gene associated with host immune response selected from the group consisting of TLR3, TLR7, TLR21, RIG-1, LPGP2, RIG-1-like receptors, TRIM25, IFNA, IFNB, IFNB1, IFNG, MAVS, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF9, IRF10, 2′,5′ oligoadenylate synthetase, RNaseL, PKR (EIF2AK2), MX1, IFITM1, IFITM2, IFITM3, Proinflammatory cytokines, Dicer, MYD88, TRIF, PKR, CSKN2B, and a regulatory region of any of the foregoing.

3. The method of paragraph 1 or 2, further comprising introducing into the egg a second RNA effector molecule targeting a second target gene, wherein the second target gene is a gene associated with cell viability, growth or cell cycle, selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STK17A, APITD1, SIVAL FAS, TGF132, TGFBR1, LOC378902, BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, CSKN2B, a miRNA antagonist, host sialidase, NEU2 sialidase 2, NEU3 sialidase 3, Dicer, ISRE, B4GalT1, B4Galt6, Cmas, Gne, SL35A1, and a regulatory region of any of the foregoing.

4. The method of any one of paragraphs 1 to 3, further comprising introducing into the egg a RNA effector molecule targeting a target gene of an endogenous virus, a latent virus, or and adventitious virus.

5. The method of any one of paragraphs 1 to 4, further comprising introducing into the egg a RNA effector molecule targeting a target gene that is a viral gene selected from influenza NP, PA, PB1, PB2, M, NS, HA, NA, genes affecting the glycolsylation of HA or NA, and a regulatory region of any of the foregoing.

6. The method of paragraph 1, wherein the RNA effector molecule inhibits or activates gene expression.

7. The method of paragraph 1, wherein the modulated gene expression increases intra-ovum viral infectivity, viral replication, cell viability, cell growth, translation, protein production, or viral adsorption.

8. The method of paragraph 1, wherein modulating gene expression decreases apoptosis in infected cells.

9. The method of paragraph 1, wherein the RNA effector molecule comprises an oligonucleotide.

10. The method of paragraph 9, wherein the oligonucleotide is a single-stranded or double-stranded oligonucleotide.

11. The method of paragraph 9 or 10 wherein the oligonucleotide is modified.

12. The method of paragraph 11, wherein the modification is selected from the group consisting of: 2′-O-methyl modified nucleotide, a nucleotide having a 5′-phosphorothioate group, a terminal nucleotide linked to a cholesteryl derivative, a 2′-deoxy-2′-fluoro modified nucleotide, a 2′-deoxy-modified nucleotide, a locked nucleotide (LNA), an abasic nucleotide, 2′-amino-modified nucleotide, 2′-alkyl-modified nucleotide, morpholino nucleotide, a phosphoramidate, a peptide nucleic acid (PNA), and a non-natural base comprising nucleotide.

13. The method of any one of the preceding paragraphs, wherein the oligonucleotide comprises an siRNA, a miRNA, a shRNA, a ribozyme, an antisense RNA, a decoy oligonucleotide, an antimir, a supermir, or a RNA activator.

14. The method of any one of the preceding paragraphs, further comprising administering to the embryonated egg a second agent selected from an immunosuppressive agent, a growth factor, an apoptosis inhibitor, a kinase inhibitor, a phosphatase inhibitor, a protease inhibitor, an inhibitor of pathogens, and a histone demethylating agent.

15. The method of any one of the preceding paragraphs, wherein the RNA effector molecule is formulated.

16. The method of paragraph 15, wherein the RNA effector molecule is formulated in a lipid particle.

17. The method of paragraph 16, wherein the lipid particle is a XTC-MC3-C12-200-based lipid particle.

18. A method for producing a biological product in an embryonated egg, comprising:

(a) introducing into the egg at least a first RNA effector molecule, a portion of which is complementary to at least a first target gene, and a second RNA effector molecule, a portion of which is complementary to at least a second target gene;

(b) maintaining the egg for a time sufficient to modulate expression of the at least one of the first and second target genes, and

(c) isolating the biological product from the egg;

wherein the first target gene is a gene of a cellular immune response, and the second target gene is a gene of a cellular process.

19. The method of paragraph 18, wherein the target gene is a gene associated with host immune response selected from the group consisting of TLR3, TLR7, TLR21, RIG-1, LPGP2, RIG-1-like receptors, TRIM25, IFNα, IFNB, IFNB1, IFNG, MAVS, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF9, IRF10, 2′,5′ oligoadenylate synthetase, RNaseL, PKR (EIF2AK2), MX1, IFITM1, IFITM2, IFITM3, Proinflammatory cytokines, Dicer, MYD88, TRIF, PKR, CSKN2B, and a regulatory region of any of the foregoing.

20. The method of paragraph 18, wherein the second target gene is a gene associated with cell viability, growth or cell cycle, selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STK17A, APITD1, SIVA1, FAS, TGFβ2, TGFBR1, LOC378902, BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, CSKN2B, a miRNA antagonist, host sialidase, NEU2 sialidase 2, NEU3 sialidase 3, Dicer, ISRE, B4GalT1, B4Galt6, Cmas, Gne, SL35A1, and a regulatory region of any of the foregoing.

21. The method of paragraphs 18 to 20, further comprising contacting the egg with a RNA effector molecule wherein the target gene is a viral gene selected from influenza NP, PA, PB1, PB2, M, NS, HA, NA, genes affecting the glycolsylation of HA or NA, and a regulatory region of any of the foregoing.

22. The method of paragraph 18, wherein the RNA effector molecule inhibits or activates gene expression.

23. The method of paragraph 18, wherein the modulated gene expression increases intra-ovum viral infectivity, viral replication, cell viability, cell growth, translation, protein production, or viral adsorption.

24. The method of paragraph 18, wherein modulating gene expression decreases apoptosis in infected cells.

25. The method of paragraph 18, wherein the RNA effector molecule comprises an oligonucleotide.

26. The method of paragraph 25, wherein the oligonucleotide is a single-stranded or double-stranded oligonucleotide.

27. The method of paragraph 25 or 26 wherein the oligonucleotide is modified.

28. The method of paragraph 27, wherein the modification is selected from the group consisting of: 2′-O-methyl modified nucleotide, a nucleotide having a 5′-phosphorothioate group, a terminal nucleotide linked to a cholesteryl derivative, a 2′-deoxy-2′-fluoro modified nucleotide, a 2′-deoxy-modified nucleotide, a locked nucleotide (LNA), an abasic nucleotide, 2′-amino-modified nucleotide, 2′-alkyl-modified nucleotide, morpholino nucleotide, a phosphoramidate, a peptide nucleic acid (PNA), and a non-natural base comprising nucleotide.

29. The method of any one of paragraphs 18 to 28, wherein the oligonucleotide comprises an siRNA, a miRNA, a shRNA, a ribozyme, an antisense RNA, a decoy oligonucleotide, an antimir, a supermir, or a RNA activator.

30. The method of any one of paragraphs 18 to 29, further comprising administering to the embryonated egg a second agent selected from an immunosuppressive agent, a growth factor, an apoptosis inhibitor, a kinase inhibitor, a phosphatase inhibitor, a protease inhibitor, an inhibitor of pathogens, and a histone demethylating agent.

31. The method of any one of paragraphs 18 to 30, wherein the RNA effector molecule is formulated.

32. The method of paragraph 31, wherein the RNA effector molecule is formulated in a lipid particle.

33. The method of paragraph 32, wherein the lipid particle is a XTC-MC3-C12-200-based lipid particle.

34. An immunogenic agent produced by the process comprising:

(a) introducing into an embryonated egg at least a first RNA effector molecule, a portion of which is complementary to at least a first target gene, and a second RNA effector molecule, a portion of which is complementary to at least a second target gene;

(b) maintaining the egg for a time sufficient to modulate expression of the at least the first and second target genes, wherein the modulation of expression improves production of a biological product in the egg; and

(c) isolating the immunogenic product from the egg;

wherein the first target gene is a gene of an immune response, and the second target gene is a gene of a cellular process.

35. The immunogenic agent of paragraph 34, wherein the immunogenic agent is viral product and is immunogenic against influenza, measles, mumps, rubella, yellow fever, rabies, small pox, chicken pox, west nile virus, cancer, hepatits, Newcastle disease, avian pox, duck plague, avian encephalomyelitis, egg drop syndrome, infectious bronchitis, Marek's disease, infectious bursal disease, infectious laryngotracheitis, or rinderpest.

36. The immunogenic agent of paragraph 35, wherein the product is immunogenic against influenza.

37. The immunogenic agent of paragraph 36, wherein the immunogenic agent contains a viral titre of at least 10⁶ EID₅₀ (50% Embryo Infective Dose) per dose.

38. The immunogenic agent of any one of the preceding paragraphs, wherein the first target gene is a gene associated with an egg cell immune response, selected from the group consisting of TLR3, TLR7, TLR21, RIG-1, LPGP2, RIG-1-like receptors, TRIM25, IFNA, IFNB, IFNG MAVS/VISA/IPS-1/Gardif, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF 9, IRF10, 2′,5′ oligoadenylate synthetase, RNaseL, dsRNA-dPKR, Mx, IFITM1, IFITM2, IFITM3, Proinflammatory cytokines, MYD88, TRIF, Dicer, PKR, CSKN2B, and a regulatory region of any of the foregoing.

39. The immunogenic agent of any one of the preceding paragraphs, wherein the second target gene is a gene associated with egg cell viability, growth or cell cycle, selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STK17A, APITD1, SIVA1, FAS, TGF132, TGFBR1, LOC378902, or BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, a miRNA antagonist, host sialidase, NEU2 sialidase 2, NEU3 sialidase 3, Dicer, ISRE, B4GalT1, B4GalT6, Cmas, Gne, SL35A1, and a regulatory region of any of the foregoing.

40. The immunogenic agent of any one of the preceding paragraphs, wherein the cell is contacted with a RNA effector molecule wherein the target gene affects the glycolsylation of HA or NA.

41. The immunogenic agent of any one of the preceding paragraphs, wherein the cell is contacted with a RNA effector molecule wherein the target gene is a viral gene selected from NP, PA, PB1, PB2, M, NS, HA, NA, or a regulatory region of any of the foregoing.

42. The immunogenic agent of any one of the preceding paragraphs, wherein the RNA effector molecule inhibits or activates gene expression.

43. The immunogenic agent of any one of the preceding paragraphs, wherein the modulated gene expression increases intra-ovum viral infectivity, viral replication, cell viability, cell growth, translation, protein production, or viral adsorption.

44. The immunogenic agent of any one of the preceding paragraphs, wherein modulating gene expression decreases apoptosis in infected cells.

45. The immunogenic agent of any one of the preceding paragraphs, wherein the RNA effector molecule comprises an oligonucleotide.

46. The immunogenic agent of any one of the preceding paragraphs, wherein the oligonucleotide is a single-stranded or double-stranded oligonucleotide.

47. The immunogenic agent of paragraph 45 or 46 wherein the oligonucleotide is modified.

48. The immunogenic agent of paragraph 47, wherein the modification is selected from the group consisting of: 2′-O-methyl modified nucleotide, a nucleotide having a 5′-phosphorothioate group, a terminal nucleotide linked to a cholesteryl derivative, a 2′-deoxy-2′-fluoro modified nucleotide, a 2′-deoxy-modified nucleotide, a locked nucleotide (LNA), an abasic nucleotide, 2′-amino-modified nucleotide, 2′-alkyl-modified nucleotide, morpholino nucleotide, a phosphoramidate, a peptide nucleic acid (PNA), and a non-natural base comprising nucleotide.

49. The immunogenic agent of any one of paragraphs 34 to 48, wherein the oligonucleotide comprises an siRNA, a miRNA, a shRNA, a ribozyme, an antisense RNA, a decoy oligonucleotide, an antimir, a supermir, or a RNA activator. 50. The immunogenic agent of any one of paragraphs 34 to 49, further comprising administering to the embryonated egg a second agent selected from an immunosuppressive agent, a growth factor, an apoptosis inhibitor, a kinase inhibitor, a phosphatase inhibitor, a protease inhibitor, an inhibitor of pathogens, and a histone demethylating agent.

51. The immunogenic agent of any one of paragraphs 34 to 50, wherein the RNA effector molecule is formulated.

52. The immunogenic agent of paragraph 51, wherein the RNA effector molecule is formulated in a lipid particle.

53. The immunogenic agent of paragraph 52, wherein the lipid particle is a XTC-MC3-C12-200-based lipid particle.

### EXAMPLES

### Example 1

### Virus and siRNA Inoculation in Chicken Embryos

For each inoculation, 30 μl of OLIGOFECTAMINE® transfection reagent (Invitrogen, Carlsbad, Calif.) is diluted with 30 μl of Opti-MEM I medium (GIBCO). siRNA (2.5 nmol (10 μl)) is mixed with 30 μl of Opti-MEM I and added to diluted OLIGOFECTAMINE® reagent, and the mixture is incubated at room temperature for 30 min. The mixture is then combined with 100 μl of influenza virus (5,000 plaque-forming units (pfu)/ml) and immediately injected into the allantoic cavity of 10-day-old embryonated chicken eggs. The eggs are incubated at 37° C. for 17 hr and 48 hr, and allantoic fluid is harvested to measure virus titer, for, example by hemagglutination or plaque assays. See Ge et al., 100 PNAS 2718-23 (2003).

1. A method for producing a biological product in an embryonated egg, comprising:
(a) introducing into the egg at least a first RNA effector molecule, a portion of which is complementary to a target gene;
(b) maintaining the egg for a time sufficient to modulate expression of the at least one of the first and second target genes, and
(c) isolating the biological product from the egg; wherein the target gene is a gene of a cellular immune response.

2. The method of claim 1, wherein the target gene is a gene associated with host immune response selected from the group consisting of TLR3, TLR7, TLR21, RIG-1, LPGP2, RIG-1-like receptors, TRIM25, IFNA, IFNB, IFNB1, IFNG, MAVS, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF9, IRF 10, 2′,5′ oligoadenylate synthetase, RNaseL, PKR (EIF2AK2), MX1, IFITM1, IFTM2, IFITM3, Proinflammatoly cytokines, Dicer, MYD88, TRIF, PKR, CSKN2B, and a regulatory region of any of the foregoing.

3. The method of claim 1, further comprising introducing into the egg a second RNA effector molecule targeting a second target gene, wherein the second target gene is a gene associated with cell viability, growth or cell cycle, selected from the group consisting of Bax, Bak, LDHA, LDHB, BIK, BAD, BIM, FMK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, BCL2L13, BCL2L14, FASLG, DPF2, AIFM2, AIFM3, STKI7A, APITD1, SIVA1, FAS, TGFβ2, TGFBR1, LOC378902, BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, CSKN2B, a miRNA antagonist, host sialidase, NEU2 sialidase 2, NEU3 sialidase 3, Dicer, ISRE, B4GalT1, B4Galt6, Cmas, Gne, SL35A1, and a regulatory region of any of the foregoing.

4. The method of claim 1, further comprising introducing into the egg a RNA effector molecule targeting a target gene of an endogenous virus, a latent virus, or and adventitious virus.

5. The method of claim 1, further comprising introducing into the egg a RNA effector molecule targeting a target gene that is a viral gene selected from influenza NP, PA, PB1, PB2, M, NS, HA, NA, genes affecting the glycolsylation of HA or NA, and a regulatory region of any of the thregoing.

6. The method of claim 1, wherein the RNA effector molecule inhibits or activates gene expression.

7. The method of claim 1, wherein the modulated gene expression increases intra-ovum viral infectivity, viral replication, cell viability, cell growth, translation, protein production, or viral adsorption.

8. The method of claim 1, wherein modulating gene expression decreases apoptosis in infected cells.

9. The method of claim 1, wherein the RNA effector molecule comprises an oligonucleotide.

10. The method of claim 9, wherein the oligonucleotide is a single-stranded or double-stranded oligonucleotide.

11. The method of claim 10 wherein the oligonucleotide is modified.

12. The method of claim 11, wherein the modification is selected from the group consisting of: 2′-O-methyl modified nucleotide, a nucleotide having a 5′-phosphorothioate group, a terminal nucleotide linked to a cholesteryl derivative, a 2′-deoxy-2′-fluoro modified nucleotide, a 2′-deoxy-modified nucleotide, a locked nucleotide (LNA), an abasic nucleotide, 2′-amino-modified nucleotide, 2′-alkyl-modified nucleotide, morpholino nucleotide, a phosphoramidate, a peptide nucleic acid (PNA), and a non-natural base comprising nucleotide.

13. The method of claim 11, wherein the oligonucleotide comprises an siRNA, a miRNA, a shRNA, a ribozyme, an antisense RNA, a decoy oligonucleotide, an antimir, a supermir, or a RNA activator.

14. The method of claim 1, further comprising administering to the embryonated egg a second agent selected from an immunosuppressive agent, a growth factor, an apoptosis inhibitor, a kinase inhibitor, a phosphatase inhibitor, a protease inhibitor, an inhibitor of pathogens, and a histone demethylating agent.

15. The method of claim 13, wherein the RNA effector molecule is formulated.

16. The method of claim 15, wherein the RNA effector molecule is formulated in a lipid particle.

17. The method of claim 16, wherein the lipid particle is a XTC-MC3-C12-200-based lipid particle.

18. A method for producing a biological product in an embryonated egg, comprising:
(a) introducing into the egg at least a first RNA effector molecule, a portion of which is complementary to at least a first target gene, and a second RNA effector molecule, a portion of which is complementary to at least a second target gene;
(b) maintaining the egg for a time sufficient to modulate expression of the at least one of the first and second target genes, and
(c) isolating the biological product from the egg;
wherein the first target gene is a gene of a cellular immune response, and the second target gene is a gene of a cellular process.

19. The method of claim 18, wherein the target gene is a gene associated with host immune response selected from the group consisting of TLR3, TLR7, TLR21, RIG-1, LPGP2, RIG-1-like receptors, TRIM25, IFNA, IFNB, IFNB1, IFNG, MAVS, IFNAR1, IFNR2, STAT-1, STAT-2, STAT-3, STAT-4, JAK-1, JAK-2, JAK-3, IRF1, IRF2, IRF3, IRF4, IRF5, IRF6 IRF7, IRF8, IRF9, IRF10, 2′,5′ oligoadenylate synthetase, RNaseL, PKR (EIF2AK2), MX1, IFITM1, IFITM2, IFITM3, Proinflammatory cytokines, Dicer, MYD88, TRIF, PKR, CSKN2B, and a regulatory region of any of the foregoing.

20. The method of claim 18, wherein the second target gene is a gene associated. with cell viability. growth or cell cycle, selected from the group consisting of Bax, Bak, LDHA, LDHB, BIK, BAD, BIM, HRK, BCLG, HR, NOXA, PUMA, BOK, BOO, BCLB, CASP2, CASP3, CASP6, CASP7, CASP8, CASP9, CASP10, BCL2, p53, APAF1, HSP70, TRAIL, BCL2L1, HCL2L13, BCL2L14, FASLG, DPF2, AIFM2AIFM3, STK17A, APITD1, SIVA1, FAS, TGβ2, TGFBR1, LOC378902, BCL2A1, PUSL1, TPST1, WDR33, Nod2, MCT4, ACRC, AMELY, ATCAY, ANP32B, DEFA3, DHRS10, DOCK4, FAM106A, FKBP1B, IRF3, KBTBD8, KIAA0753, LPGAT1, MSMB, NFS1, NPIP, NPM3, SCGB2A1, SERPINB7, SLC16A4, SPTBN4, TMEM146, CDKN1B, CDKN2A, FOXO1, PTEN, FN1, CSKN2B, a miRNA antagonist, host sialidase, NEU2 sialidase 2, NEU3 sialidase 3, Dicer, ISRE, B4GalT1, B4Galt6, Cmas, Gne, SL35A1, and a regulatory region of any of the foregoing.

21-53. (canceled)
